(19) United States
(12) Patent Application Publication
(10) Pub. No.: US 2020/0319205 A1
(43) Pub. Date: Oct. 8, 2020

(54) GENE CLASSIFIERS FOR USE IN MONITORING UV DAMAGE

(71) Applicant: DermTech, Inc., La Jolla, CA (US)

(72) Inventors: John Daniel DOBAK, III, La Jolla, CA (US); Burkhard JANSEN, La Jolla, CA (US); Zuxu YAO, La Jolla, CA (US); James ROCK, La Jolla, CA (US)

(21) Appl. No.: 16/838,653

(22) Filed: Apr. 2, 2020

Related U.S. Application Data
(60) Provisional application No. 62/830,105, filed on Apr. 5, 2019.
(60) Provisional application No. 62/895,364, filed on Sep. 3, 2019.

(51) Int. Cl. G01N 33/68; G01N 33/50

(52) U.S. Cl. CPC G01N 33/6881; G01N 33/5023; G01N 33/5044

(57) ABSTRACT

Disclosed herein is a method of detecting the presence of skin UV damage based on molecular risk factors. In some instances, also described herein is a method of determining the progression of UV damage based on the molecular risk factors.

### CROSS-REFERENCE

This application claims the benefit of U.S. Provisional Application Nos. 62/830,105, filed Apr. 5, 2019, and 62/895,364, filed Sep. 3, 2019, which applications are incorporated herein by reference in their entireties.

### BACKGROUND OF THE DISCLOSURE

Skin diseases are some of the most common human illnesses and represent an important global burden in healthcare. Three skin diseases are in the top ten most prevalent diseases worldwide, and eight fall into the top 50. When considered collectively, skin conditions range from being the second to the 11th leading causes of years lived with disability.

### SUMMARY OF THE DISCLOSURE

Disclosed herein, in certain embodiments, is a method of assessing or detecting the presence of UV damage based on molecular risk factors. Some embodiments include various uses of the UV damage determination. In some instances, also described herein is a method of determining the progression of UV damage based on the molecular risk factors.

Disclosed herein, in certain embodiments, is a method of detecting gene expression level of cellular retinoic acid binding protein 2 (CRABP2), interleukin 1 receptor antagonist (IL1RN), interleukin-36 gamma (IL36G), small breast epithelial mucin (MUCL1), programmed cell death 4 (PDCD4), small proline-rich protein 1A (SPRR1A), cystatin E/M (CST6), kallikrein related peptidase 10 (KLK10), or a combination thereof in a subject in need thereof, comprising: (a) isolating nucleic acids from a skin sample obtained from the subject, wherein the skin sample comprises cells from the stratum corneum; and (b) detecting the expression level of CRABP2, IL1RN, IL36G, MUCL1, PDCD4, SPRR1A, CST6, KLK10, or a combination thereof, by contacting the isolated nucleic acids with a set of probes that recognizes CRABP2, IL1RN, IL36G, MUCL1, PDCD4, SPRR1A, CST6, KLK10, or a combination thereof, and detects binding between CRABP2, IL1RN, IL36G, MUCL1, PDCD4, SPRR1A, CST6, KLK10, or a combination thereof and the set of probes. In some embodiments, the method comprises detecting the expression levels of SPRR1A and MUCL1. In some embodiments, the method comprises detecting the expression levels of CRABP2, IL36G, MUCL1, PDCD4, and CST6. In some embodiments, the method comprises detecting the expression levels of IL1RN, SPRR1A, and KLK10. In some embodiments, the method comprises detecting the expression levels of IL1RN and IL36G. In some embodiments, the method comprises detecting the expression levels of CRABP2, MUCL1, PDCD4, SPRR1A, CST6, and KLK10. In some embodiments, the method comprises detecting the expression levels of CRABP2, IL1RN, IL36G, MUCL1, PDCD4, SPRR1A, CST6, and KLK10. In some embodiments, the expression level is a down-regulated gene expression level, compared to a gene expression level of an equivalent gene from a control sample. In some embodiments, the gene expression level of CRABP2, MUCL1, PDCD4, SPRR1A, CST6, or KLK10 is down-regulated. In some embodiments, the expression level is an up-regulated gene expression level, compared to a gene expression level of an equivalent gene from a control sample. In some embodiments, the gene expression level of IL1RN or IL36G is up-regulated. In some embodiments, the set of probes recognizes at least one but no more than eight genes. In some embodiments, the method further comprises detecting the expression levels of IL22RA1, IL36B, KRT17, ADAMTSL4, CDKN1A, KIF18B, MKI67, SLAMF7, TRIP13, UHRF1, or a combination thereof. In some embodiments, the detecting comprises contacting the isolated nucleic acids with an additional set of probes that recognizes IL22RA1, IL36B, KRT17, ADAMTSL4, CDKN1A, KIF18B, MKI67, SLAMF7, TRIP13, UHRF1, or a combination thereof, and detects binding between IL22RA1, IL36B, KRT17, ADAMTSL4, CDKN1A, KIF18B, MKI67, SLAMF7, TRIP13, UHRF1, or a combination thereof and the additional set of probes. In some embodiments, the additional set of probes recognizes one but no more than ten genes. In some embodiments, the nucleic acids comprise RNA, DNA, or a combination thereof. In some embodiments, the RNA is mRNA. In some embodiments, the RNA is cell-free circulating RNA. In some embodiments, the cells from the stratum corneum comprises T cells or components of T cells. In some embodiments, the cells from the stratum corneum comprises keratinocytes. In some embodiments, the skin sample does not comprise melanocytes. In some embodiments, the skin sample is obtained by applying an adhesive patch to a skin region of the subject in a manner sufficient to adhere cells to the adhesive patch, and removing the adhesive patch from the skin region in a manner sufficient to retain the adhered cells to the adhesive patch. In some embodiments, the skin sample is obtained by applying a plurality of adhesive patches to a skin region of the subject in a manner sufficient to adhere cells to each of the adhesive patches, and removing each of the adhesive patches from the skin region in a manner sufficient to retain the adhered cells to each of the adhesive patches. In some embodiments, the plurality of adhesive patches comprises at least 4 adhesive patches. In some embodiments, the skin region is a skin lesion region. In some embodiments, the skin region of the subject has UV damage. In some embodiments, the subject is treated with T4 endonuclease V-based treatment or photolyase-based treatment. In some embodiments, the expression level of genes is monitored over the course of 1 week, 2 weeks, 3 weeks, 1 month, 2 months, 6 months, or more. In some embodiments, the subject is a human.

Disclosed herein, in certain embodiments, is a method of detecting gene expression levels from a first gene classifier and a second gene classifier in a subject in need thereof, comprising: (a) isolating nucleic acids from a skin sample obtained from the subject, wherein the skin sample comprises cells from the stratum corneum; (b) detecting the expression levels of one or more genes from the first gene classifier: CRABP2, IL1RN, IL36G, MUCL1, PDCD4, SPRR1A, CST6, and KLK10, by contacting the isolated nucleic acids with a set of probes that recognizes one or more genes from the first gene classifier, and detects binding between one or more genes from the first gene classifier and the set of probes; and (c) detecting the expression levels of one or more genes from the second gene classifier: IL22RA1, IL36B, KRT17, ADAMTSL4, CDKN1A, KIF18B, MKI67, SLAMF7, TRIP13, and UHRF1, by contacting the isolated nucleic acids with an additional set of probes that recognizes one or more genes from the second gene classifier, and detects binding between one or more genes from the second gene classifier and the additional set of probes. In some embodiments, the method comprises detecting the expression levels of SPRR1A and MUCL1 from the first gene classifier. In some embodiments, the method comprises detecting the expression levels of CRABP2, IL36G, MUCL1, PDCD4, and CST6 from the first gene classifier. In some embodiments, the method comprises detecting the expression levels of IL1RN, SPRR1A, and KLK10 from the first gene classifier. In some embodiments, the method comprises detecting the expression levels of IL1RN and IL36G from the first gene classifier. In some embodiments, the method comprises detecting the expression levels of CRABP2, MUCL1, PDCD4, SPRR1A, CST6, and KLK10 from the first gene classifier. In some embodiments, the method comprises detecting the expression levels of CRABP2, IL1RN, IL36G, MUCL1, PDCD4, SPRR1A, CST6, and KLK0 from the first gene classifier. In some embodiments, the expression level is a down-regulated gene expression level, compared to a gene expression level of an equivalent gene from a control sample. In some embodiments, the gene expression level of CRABP2, MUCL1, PDCD4, SPRR1A, CST6, or KLK10 is down-regulated. In some embodiments, the expression level is an up-regulated gene expression level, compared to a gene expression level of an equivalent gene from a control sample. In some embodiments, the gene expression level of IL1RN or IL36G is up-regulated. In some embodiments, the set of probes recognizes at least one but no more than eight genes. In some embodiments, the additional set of probes recognizes one but no more than ten genes. In some embodiments, the nucleic acids comprise RNA, DNA, or a combination thereof. In some embodiments, the RNA is mRNA. In some embodiments, the RNA is cell-free circulating RNA. In some embodiments, the cells from the stratum corneum comprises T cells or components of T cells. In some embodiments, the cells from the stratum corneum comprises keratinocytes. In some embodiments, the skin sample does not comprise melanocytes. In some embodiments, the skin sample is obtained by applying an adhesive patch to a skin region of the subject in a manner sufficient to adhere cells to the adhesive patch, and removing the adhesive patch from the skin region in a manner sufficient to retain the adhered cells to the adhesive patch. In some embodiments, the skin sample is obtained by applying a plurality of adhesive patches to a skin region of the subject in a manner sufficient to adhere cells to each of the adhesive patches, and removing each of the adhesive patches from the skin region in a manner sufficient to retain the adhered cells to each of the adhesive patches. In some embodiments, the plurality of adhesive patches comprises at least 4 adhesive patches. In some embodiments, the skin region is a skin lesion region. In some embodiments, the skin region of the subject has UV damage. In some embodiments, the subject is treated with T4 endonuclease V-based treatment or photolyase-based treatment. In some embodiments, the expression level of genes is monitored over the course of 1 week, 2 weeks, 3 weeks, 1 month, 2 months, 6 months, or more. In some embodiments, the subject is a human.

Disclosed herein, in certain embodiments, is a method of administering a DNA repair enzyme to a subject in need thereof. In some embodiments, the subject is suffering from a sunburn. In some embodiments, the method modulates gene or protein expression in the subject.

Disclosed herein, in certain embodiments, are methods of determining the presence of UV skin damage. Disclosed herein, in certain embodiments, are methods of identifying a subject with UV skin damage. Disclosed herein, in certain embodiments, are methods of measuring UV skin damage. Disclosed herein, in certain embodiments, are methods of assessing the extent of UV skin damage. Some embodiments include identifying a subject suspected of having UV skin damage. Some embodiments include isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum. Some embodiments include detecting an expression level of at least one target gene known to be upregulated or downregulated in subjects with UV skin damage, by contacting the isolated nucleic acids with a set of probes that recognize the target gene, and detecting binding between the at least one target gene and the set of probes. In some embodiments, the nucleic acids comprise mRNA. In some embodiments, the cells from the stratum corneum comprise T cells or components of T cells. In some embodiments the cells from the stratum corneum comprise keratinocytes. In some embodiments, the skin sample does not comprise melanocytes. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region does not comprise a skin lesion. Some embodiments include determining whether the subject has UV skin damage based on the expression level of the at least one target gene. Some embodiments include determining the extent of UV skin damage based on the expression level of the at least one target gene. Some embodiments include administering a skin damage treatment to the subject based on the determination of whether the subject has UV skin damage. In some embodiments, the subject has UV skin damage. In some embodiments, the subject is a human. In some embodiments, the expression level is upregulated compared to a gene expression level of an equivalent gene from a control sample. Some embodiments include determining that the subject has an extent of UV skin damage above a threshold amount, based on the expression level of the at least one target gene. In some embodiments, the expression level is downregulated compared to a gene expression level of an equivalent gene from a control sample. Some embodiments include administering a UV skin damage treatment to the subject when the subject is determined to have an extent of UV skin damage above the threshold amount. Some embodiments include not administering the UV skin damage treatment to the subject when the subject is determined to have an extent of UV skin damage below the threshold amount. In some embodiments, the at least one target gene comprises a Vitamin A gene family or family member, a Programmed Cell Death Protein gene family or family member, a Small Proline Rich Protein gene family or family member, an Interleukin 1/2 gene family or family member, a cystatin gene family or family member, or a combination thereof. In some embodiments, the at least one target gene comprises ADAMTSL4, CDKN1A, CST6, KIF18B, MKI67, SLAMF7, TRIP13, UHRF1, CRABP2, IL1RN, IL22RA1, IL36B, IL36G, KLK10, KRT17, MUCL1, PDCD4, or SPRR1A, or a combination thereof.

Disclosed herein, in certain embodiments, is a method of treating a subject with UV skin damage. Some embodiments include identifying a subject suspected of having UV skin damage. Some embodiments include isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum. Some embodiments include detecting an expression level of at least one target gene known to be upregulated or downregulated in subjects with UV skin damage, by contacting the isolated nucleic acids with a set of probes that recognize the target gene, and detecting binding between the at least one target gene and the set of probes. Some embodiments include determining whether the subject has UV skin damage based on the expression level of the at least one target gene. Some embodiments include administering a UV skin damage treatment to the subject when the subject is determined to have UV skin damage based on the expression level of the at least one target gene. Some embodiments include not administering the UV skin damage treatment to the subject when the subject is not determined to have UV skin damage based on the expression level of the at least one target gene. In some embodiments, the nucleic acids comprise mRNA. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region does not comprise a skin lesion. Some embodiments include determining that the subject has UV skin damage based on the expression level of the at least one target gene. Some embodiments include determining that the subject has an extent of UV skin damage above a threshold amount, based on the expression level of the at least one target gene. Some embodiments include determining that the subject has an extent of UV skin damage below a threshold amount, based on the expression level of the at least one target gene. Some embodiments include administering a UV skin damage treatment to the subject when the subject is determined to have an extent of UV skin damage above the threshold amount. Some embodiments include not administering the UV skin damage treatment to the subject when the subject is determined to have an extent of UV skin damage below the threshold amount. In some embodiments, the at least one target gene comprises a Vitamin A gene family or family member, a Programmed Cell Death Protein gene family or family member, a Small Proline Rich Protein gene family or family member, an Interleukin 1/2 gene family or family member, a cystatin gene family or family member, or a combination thereof. In some embodiments, the at least one target gene comprises ADAMTSL4, CDKN1A, CST6, KIF18B, MKI67, SLAMF7, TRIP13, UHRF1, CRABP2, IL1RN, IL22RA1, IL36B, IL36G, KLK10, KRT17, MUCL1, PDCD4, or SPRR1A, or a combination thereof.

Disclosed herein, in certain embodiments, is a method of assessing ultraviolet (UV) skin damage, including identifying a subject exposed to UV radiation; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and measuring or detecting an expression level of at least one target gene known to be upregulated or downregulated in subjects with UV skin damage, by contacting the isolated nucleic acids with a set of probes that recognize the target gene, and measuring or detecting binding between the at least one target gene and the set of probes. In some embodiments, the UV radiation comprises UVB rays. In some embodiments, the subject is a human. In some embodiments, the nucleic acids comprise mRNA. In some embodiments, the cells from the stratum corneum comprise keratinocytes. In some embodiments, the nucleic acids are amplified prior to being contacted with the set of probes. In some embodiments, the adhesive patch comprises a rubber adhesive on a polyurethane film. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a sunburn. Some embodiments include determining whether the skin sample has UV skin damage based on the expression level of the at least one target gene. Some embodiments include administering a skin treatment to the skin region of the subject based on the determination of whether the subject has UV skin damage. Some embodiments include determining that the subject has an extent of UV skin damage above a threshold amount, based on the expression level of the at least one target gene. Some embodiments include determining that the subject has an extent of UV skin damage below a threshold amount, based on the expression level of the at least one target gene. Some embodiments include administering a UV skin damage treatment to the subject when the subject is determined to have an extent of UV skin damage above the threshold amount. Some embodiments include not administering the UV skin damage treatment to the subject when the subject is determined to have an extent of UV skin damage below the threshold amount. In some embodiments, the skin treatment is topical. In some embodiments, the skin treatment comprises a T4 endonuclease V-based treatment or photolyase-based treatment. In some embodiments, the skin sample has UV skin damage. In some embodiments, the expression level is upregulated compared to a control. In some embodiments, the expression level is downregulated compared to a control. In some embodiments, the at least one target gene comprises of a Vitamin A gene family or family member, a Programmed Cell Death Protein gene family or family member, a Small Proline Rich Protein gene family or family member, an Interleukin 1/2 gene family or family member, a cystatin gene family or family member, or a combination thereof. In some embodiments, the expression level of genes is monitored over the course of 1 week, 2 weeks, 3 weeks, 1 month, 2 months, 6 months, or more.

Disclosed herein, in certain embodiments, is a method of treating a subject with ultraviolet (UV) skin damage including identifying a subject exposed to UV radiation; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; measuring or detecting an expression level of at least one target gene known to be upregulated or downregulated in subjects with UV skin damage, by contacting the isolated nucleic acids with a set of probes that recognize the target gene, and measuring or detecting binding between the at least one target gene and the set of probes; determining whether the subject has UV skin damage based on the expression level of the at least one target gene; and administering a skin treatment to the subject when the subject is determined to have UV skin damage based on the expression level of the at least one target gene, and not administering the skin treatment to the subject when the subject is not determined to have UV skin damage based on the expression level of the at least one target gene. In some embodiments, the determination of whether the subject has UV skin damage is based on comparing the expression level(s) of the at least one target gene to a threshold amount of expression. In some embodiments, the UV radiation comprises UVB rays. In some embodiments, the subject is a human. In some embodiments, the nucleic acids comprise mRNA. In some embodiments, the cells from the stratum corneum comprise keratinocytes. In some embodiments, the nucleic acids are amplified prior to being contacted with the set of probes. In some embodiments, the adhesive patch comprises a rubber adhesive on a polyurethane film. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a sunburn. In some embodiments, the skin treatment is topical. In some embodiments, the skin treatment comprises a T4 endonuclease V-based treatment or photolyase-based treatment. In some embodiments, the skin sample has UV skin damage. In some embodiments, the expression level is upregulated compared to a control. In some embodiments, the expression level is downregulated compared to a control. In some embodiments, the at least one target gene comprises of a Vitamin A gene family or family member, a Programmed Cell Death Protein gene family or family member, a Small Proline Rich Protein gene family or family member, an Interleukin 1/2 gene family or family member, a cystatin gene family or family member, or a combination thereof. In some embodiments, the expression level of genes is monitored over the course of 1 week, 2 weeks, 3 weeks, 1 month, 2 months, 6 months, or more.

Disclosed herein, in certain embodiments, is a method of assessing ultraviolet (UV) skin damage, comprising: obtaining expression levels of target genes in a skin sample obtained from a subject; generating a UV exposure score for the subject by comparing the expression levels to a model derived from target gene expression levels in skin samples from a cohort of subjects, and derived from amounts UV skin damage or exposure in the cohort of subjects. In some embodiments, the model comprises a random forest model, a boosting model, a lasso model, or a logistic model. Some embodiments include identifying a subject exposed to UV radiation or suspected of having UV skin damage. In some embodiments, the expression levels have been obtained by isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; measuring or detecting the expression levels of the target genes, wherein the target genes are known to be upregulated or downregulated in subjects with UV skin damage, by contacting the isolated nucleic acids with a set of probes that recognize the target gene, and measuring or detecting binding between the at least one target gene and the set of probes. Some embodiments include determining whether the subject has UV skin damage based on the UV exposure score. Some embodiments include determining an amount or extent UV skin damage based on the UV exposure score. Some embodiments include administering a skin treatment to the subject. Some embodiments include administering a skin treatment to the subject based on the UV exposure score. In some embodiments, the determination of whether the subject has UV skin damage is based on comparing the UV exposure score for the subject to a threshold UV exposure score. In some embodiments, the UV radiation comprises UVB rays. In some embodiments, the subject is a human. In some embodiments, the nucleic acids comprise mRNA. In some embodiments, the cells from the stratum corneum comprise keratinocytes. In some embodiments, the nucleic acids are amplified prior to being contacted with the set of probes. In some embodiments, the adhesive patch comprises a rubber adhesive on a polyurethane film. In some embodiments, the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a sunburn. In some embodiments, the skin treatment is topical. In some embodiments, the skin treatment comprises a T4 endonuclease V-based treatment or photolyase-based treatment. In some embodiments, the skin sample has UV skin damage. In some embodiments, the skin sample has an amount of UV skin damage corresponding to the UV exposure score. In some embodiments, the expression level of one or more of the target genes is upregulated compared to a control. In some embodiments, the expression level of one or more of the target genes is downregulated compared to a control. In some embodiments, the target genes comprises a Vitamin A gene family or family member, a Programmed Cell Death Protein gene family or family member, a Small Proline Rich Protein gene family or family member, an Interleukin 1/2 gene family or family member, or a cystatin gene family or family member, or a combination thereof. In some embodiments, the expression level of genes is monitored over the course of 1 week, 2 weeks, 3 weeks, 1 month, 2 months, 6 months, or more, and multiple UV exposure scores are calculated over the course.

Disclosed herein, in certain embodiments, is a method of monitoring ultraviolet (UV) skin damage, comprising: isolating nucleic acids from a first skin sample obtained from a subject at a first time by applying an adhesive patch to a skin region of the subject in a manner sufficient to adhere skin sample cells to the adhesive patch, and removing the adhesive patch from the first skin sample in a manner sufficient to retain the adhered skin sample cells to the adhesive patch, wherein the skin sample cells comprise cells from the stratum corneum; measuring or detecting an expression level of one or more target genes known to be upregulated or downregulated in subjects with UV skin damage, in the first skin sample; determining a presence or an amount of UV skin damage in the first skin sample based on the expression level of the one or more target genes; isolating nucleic acids from a skin sample obtained from the subject at a second time; measuring or detecting an expression level of the one or more target genes in the second skin sample; determining a presence or an amount of UV skin damage in the second skin sample based on the expression level of the one or more target genes; and comparing the presence or amount of UV skin damage in the second skin sample to the presence or amount of UV skin damage in the first skin sample. Some embodiments include providing a skin treatment to the subject after the first skin sample is obtained, and before the second skin sample is obtained. In some embodiments, the skin treatment comprises a sunscreen. Some embodiments include an efficacy of the skin treatment based on the comparison of the presence or amount of UV skin damage in the second skin sample to the presence or amount of UV skin damage in the first skin sample. Some embodiments include providing a second skin treatment to the subject after second skin sample is obtained, based on the presence or amount of UV skin damage in the second skin sample compared to the presence or amount of UV skin damage in the first skin sample.

Disclosed herein, in certain embodiments, is a kit for assessing ultraviolet (UV) skin damage, comprising an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with UV skin damage.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a statistical analysis of gene expression levels.

FIG. 2 is a plot illustrating a multivariate analysis.

FIG. 3 illustrates a statistical analysis of gene expression levels.

FIG. 4 illustrates a hypothetical distribution of UV exposure scores.

FIG. 5 is a box plot showing UV exposure scores before and after UV exposure.

FIG. 6 illustrates a distribution of UV exposure scores.

FIG. 7 shows plots of gene expression data.

FIG. 8 is a density plot of UV exposure scores.

FIG. 9 is a histogram of UV exposure scores.

FIG. 10 is a plot illustrating a multivariate analysis.

FIG. 11 is a plot illustrating a multivariate analysis.

FIG. 12 is a plot illustrating a statistical analysis.

FIG. 13 is a plot illustrating a multivariate analysis with clinical characteristics included.

FIG. 14 is a density plot of UV exposure scores.

FIG. 15 is a histogram of UV exposure scores.

FIG. 16 shows a non-limiting example of a UV assessment workflow.

### DETAILED DESCRIPTION OF THE DISCLOSURE

Ultraviolet (UV) rays present one of the greatest risk factors for developing a skin cancer. The UV rays comprise 3 main types, UVA, UVB, and UVC. About 95% of the UV radiation is UVA rays, and which penetrates deep into the skin layer, leading to DNA damage by creating free radicals via reactive oxygen species and decreasing the activity of antigen present cells of the epidermis. UVB rays, also known as sunburn rays, are generally associated with skin cancer due the ability to induce formation of cyclobutane pyrimidine dimers and pyrimidine (6-4) photoproducts.

In some embodiments, disclosed herein is a method of determining an expression change in one or more skin gene markers following exposure to UV radiation (e.g., UVB radiation). In some instances, also described herein is a method of monitoring the one or more skin gene markers for the presence of sun damage, and downstream development of a skin cancer.

In some embodiments, disclosed herein is a method of utilizing the expression level of genes in a gene classifier to determine the presence of UV skin damage. The gene classifier may be a UV exposure score. In some cases, the method comprises determining a change in the expression level of genes in a gene classifier, in which the change is compared to a gene expression level of an equivalent gene from a normal sample. In additional embodiments, disclosed herein is a method of determining whether a subject has UV skin damage based on the expression level of genes in a gene classifier.

Disclosed herein, in some embodiments, are methods of determining the presence of UV skin damage in a skin sample, comprising: identifying a subject suspected of having UV skin damage; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and detecting an expression level of at least one target gene known to be upregulated or downregulated in subjects with UV skin damage, by contacting the isolated nucleic acids with a set of probes that recognize the target gene, and detecting binding between the at least one target gene and the set of probes. Some embodiments include the use of a clinical factor in determining the presence of the UV skin damage.

Disclosed herein, in some embodiments, are methods of treating a subject with UV skin damage, comprising: identifying a subject suspected of having UV skin damage; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; detecting an expression level of at least one target gene known to be upregulated or downregulated in subjects with UV skin damage, by contacting the isolated nucleic acids with a set of probes that recognize the target gene, and detecting binding between the at least one target gene and the set of probes; determining whether the subject has UV skin damage based on the expression level of the at least one target gene; and administering a skin damage treatment such as a UV skin damage treatment to the subject when the subject is determined to have UV skin damage based on the expression level of the at least one target gene, and not administering the skin damage treatment to the subject when the subject is not determined to have UV skin damage based on the expression level of the at least one target gene. Some embodiments include the use of a clinical factor in determining the presence of the UV skin damage.

Disclosed herein, in some embodiments, are kits for determining the presence of UV skin damage in a skin sample, comprising: an adhesive patch comprising an adhesive matrix configured to adhere skin sample cells from the stratum corneum of a subject; a nucleic acid isolation reagent; and a plurality of probes. In some embodiments, the probes recognize at least one target gene known to be upregulated or downregulated in subjects with UV skin damage.

The kits and methods disclosed herein have several advantages over the prior art. An advantage of using target genes for identifying subjects with UV skin damage, or for determining the presence of UV skin damage in a skin sample, is the relatively low cost of obtaining genetic data such as information about gene expression compared to, for example, protein biomarkers. An advantage of using an adhesive tape to collect a skin sample is its non-invasiveness.

In some cases, gene expression data, such as measured amounts of mRNA of one or more target genes, are indicative of UV skin damage. Because mRNA levels do not always correlate with protein levels for a given gene, an existing method that measures protein levels would not render obvious the methods described herein. The usefulness of expression levels of the various genes and type of genes described herein is unexpected in light of such methods because of the unpredictability of whether mRNA levels and protein levels will always align. For example, in one instance a mRNA expression level for a gene may be increased in UV damaged skin compared to a control sample while the protein level of the gene may be unchanged; or vice versa, a protein level may be increased or decreased in UV damaged skin while an mRNA level for the same gene as the protein is unchanged. A benefit of using RNA-based gene expression to indicate of UV skin damage is that RNA deals with a snapshot in time, and can be used to monitor changes and repair, such as UV skin damage repair linked to cosmeceutical or therapeutic products.

The methods described herein may be used to evaluate a skin treatment regimen. For example, an adhesive patch skin collection system described herein may be used to evaluate a sunscreen based on the expression of one or more target genes, rather than evaluating the sunscreen simply based on erythema levels. Thus, the methods described herein include novel way to evaluate sunscreens and sunscreen ingredients.

### Target Genes, Gene Classifiers, and Methods of Use

Disclosed herein, in some embodiments, are methods that include measuring, detecting, or using a target gene. For example, some embodiments relate to a method of detecting, assessing, measuring, or determining the presence of a skin damage such as UV skin damage based on a presence or expression level of the target gene. Some embodiments relate to a method of identifying a subject with UV skin damage based on a presence or expression level of the target gene. Some embodiments relate to a method of identifying a subject with an amount or extent of UV skin damage based on a presence or expression level of the target gene. Some embodiments include detecting, assessing, measuring, or determining the presence of UV skin damage based on a presence or expression level of the target gene. Some embodiments include the use of multiple target genes. In some embodiments, the target genes described herein are used in any method described herein. In some embodiments, the target genes are used to rule out a skin damage other than UV skin damage. In some embodiments, the UV skin damage is caused by the sun. In some embodiments, the UV skin damage is not caused by the sun. Some embodiments include use of one or more target genes in a method described herein.

The target genes may be used to evaluate UV skin damage. Disclosed herein, in certain embodiments, are methods of determining the presence of UV skin damage. Disclosed herein, in certain embodiments, are methods of identifying a subject with UV skin damage. Disclosed herein, in certain embodiments, are methods of measuring UV skin damage. Disclosed herein, in certain embodiments, are methods of assessing the extent of UV skin damage. In some embodiments, the determination, identification, measurement, or assessment is before UV skin damage or UV exposure occurs. In some embodiments, the determination, identification, measurement, or assessment is after the UV skin damage or UV exposure. In some embodiments, the UV skin damage is acute, or the UV exposure is acute. In some embodiments, the determination, identification, measurement, or assessment is a period of time after the UV skin damage or UV exposure. In some embodiments the period of time is 6 hours, 12 hours, 18 hours, 1 day, 2 days, 3 days, 4 days, 5 days, 6 days, 1 week, 2 weeks, 3 weeks, 4 weeks, or 1 month, or a range of time defined by any two of the aforementioned time periods. In some embodiments the period of time is about 6 hours, about 12 hours, about 18 hours, about 1 day, about 2 days, about 3 days, about 4 days, about 5 days, about 6 days, about 1 week, about 2 weeks, about 3 weeks, about 4 weeks, or about 1 month. In some embodiments the period of time is at least 6 hours, at least 12 hours, at least 18 hours, at least 1 day, at least 2 days, at least 3 days, at least 4 days, at least 5 days, at least 6 days, at least 1 week, at least 2 weeks, at least 3 weeks, at least 4 weeks, or at least 1 month. In some embodiments the period of time is no more than 6 hours, no more than 12 hours, no more than 18 hours, no more than 1 day, no more than 2 days, no more than 3 days, no more than 4 days, no more than 5 days, no more than 6 days, no more than 1 week, no more than 2 weeks, no more than 3 weeks, no more than 4 weeks, or no more than 1 month. In some embodiments the period of time is 24 hours. In some embodiments the period of time is about 24 hours. In some embodiments the period of time is at least 24 hours. In some embodiments the period of time is at least about 24 hours. In some embodiments the period of time is no more than 24 hours. In some embodiments the period of time is no more than about 24 hours. In some embodiments the period of time is 2 weeks. In some embodiments the period of time is about 2 weeks. In some embodiments the period of time is at least 2 weeks. In some embodiments the period of time is at least about 2 weeks. In some embodiments the period of time is no more than 2 weeks. In some embodiments the period of time is no more than about 2 weeks. Some embodiments include multiple determinations, identifications, measurements, or assessments (e.g. to monitor UV skin damage over time).

The target genes may be used to evaluate UV skin damage during a skin treatment regimen, or may be used to evaluate the skin treatment regimen. In some embodiments, the determination, identification, measurement, or assessment is before a skin treatment such as a UV skin treatment. In some embodiments, the UV skin treatment comprises a sunscreen product. In some embodiments, the determination, identification, measurement, or assessment is after the skin treatment. In some embodiments, the UV skin damage is acute, or the UV exposure is acute. In some embodiments, the determination, identification, measurement, or assessment is a period of time after the skin treatment. In some embodiments the period of time is 6 hours, 12 hours, 18 hours, 1 day, 2 days, 3 days, 4 days, 5 days, 6 days, 1 week, 2 weeks, 3 weeks, 4 weeks, or 1 month, or a range of time defined by any two of the aforementioned time periods. In some embodiments the period of time is about 6 hours, about 12 hours, about 18 hours, about 1 day, about 2 days, about 3 days, about 4 days, about 5 days, about 6 days, about 1 week, about 2 weeks, about 3 weeks, about 4 weeks, or about 1 month. In some embodiments the period of time is at least 6 hours, at least 12 hours, at least 18 hours, at least 1 day, at least 2 days, at least 3 days, at least 4 days, at least 5 days, at least 6 days, at least 1 week, at least 2 weeks, at least 3 weeks, at least 4 weeks, or at least 1 month. In some embodiments the period of time is no more than 6 hours, no more than 12 hours, no more than 18 hours, no more than 1 day, no more than 2 days, no more than 3 days, no more than 4 days, no more than 5 days, no more than 6 days, no more than 1 week, no more than 2 weeks, no more than 3 weeks, no more than 4 weeks, or no more than 1 month. In some embodiments the period of time is 24 hours. In some embodiments the period of time is about 24 hours. In some embodiments the period of time is at least 24 hours. In some embodiments the period of time is at least about 24 hours. In some embodiments the period of time is no more than 24 hours. In some embodiments the period of time is no more than about 24 hours. In some embodiments the period of time is 2 weeks. In some embodiments the period of time is about 2 weeks. In some embodiments the period of time is at least 2 weeks. In some embodiments the period of time is at least about 2 weeks. In some embodiments the period of time is no more than 2 weeks. In some embodiments the period of time is no more than about 2 weeks. Some embodiments include multiple determinations, identifications, measurements, or assessments (e.g. to monitor UV skin damage over time during a skin treatment period).

In some embodiments, the method includes one or more target genes (e.g. 1 target gene, or multiple target genes). In some embodiments, the one or more target genes include a gene encoding a Vitamin A gene family or family member, a gene encoding a Programmed Cell Death Protein gene family or family member, a gene encoding a Small Proline Rich Protein gene family or family member, a gene encoding an Interleukin 1/2 gene family member, a gene encoding a cystatin gene family or family member, or a combination thereof.

In some embodiments, the one or more target genes include a gene encoding a retinoid response gene. In some embodiments, the retinoid response gene encodes a Vitamin A gene family member. In some embodiments, the one or more target genes include a gene encoding a Vitamin A gene family member. An example of a gene encoding a Vitamin A gene family member includes but is not limited to a gene encoding cellular retinoic acid binding protein 2 (CRABP2).

In some embodiments, the one or more target genes include a gene encoding a Programmed Cell Death Protein. An example of a Programmed Cell Death Protein gene family member includes but is not limited to a gene encoding programmed cell death 4 (PDCD4).

In some embodiments, the one or more target genes include a gene encoding a Small Proline Rich Protein gene family member. An example of a Small Proline Rich Protein gene family member includes but is not limited to a gene encoding a small proline-rich protein 1A (SPRR1A).

In some embodiments, the one or more target genes include one or more genes encoding an interleukin. In some embodiments, the genes encoding an interleukin include genes encoding an Interleukin 1/2 gene family member. In some embodiments, the one or more interleukins include an Interleukin 1 gene family member. In some embodiments, the one or more interleukins include an Interleukin 2 gene family member. Examples of genes encoding Interleukin 1/2 gene family members include but are not limited to a gene encoding interleukin 1 receptor antagonist (IL1RN) and a gene encoding interleukin-36 gamma (IL36G). An example of a cystatin gene family member includes but is not limited to a gene encoding cystatin E/M (CST6).

In some embodiments, the one or more target genes include one or more genes encoding a CDKN Family member. In some embodiments, the CDKN Family member includes CDKN1A. In some embodiments, the CDKN Family member includes CDKN2A.

Disclosed herein, in some embodiments, are one or more target genes. In some embodiments, the one or more target genes comprises an ADAMTSL Family member, a CDKN Family member, a CST Family member, a KIF Family member, a MKI Family member, a SLAM Family member, a TRIP Family member, a UHRF Family member, a Vitamin A Family member, an Interleukin Family member, a KLK Family member, a KRT Family member, a MUCL Family member, a PDCD Family member, a SPRR Family member, or a combination thereof. In some embodiments, the one or more target genes comprises an ADAMTSL Family member. In some embodiments, the one or more target genes comprises a CDKN Family member. In some embodiments, the one or more target genes comprises a CST Family member. In some embodiments, the one or more target genes comprises a KIF Family member. In some embodiments, the one or more target genes comprises a MKI Family member. In some embodiments, the one or more target genes comprises a SLAM Family member. In some embodiments, the one or more target genes comprises a TRIP Family member. In some embodiments, the one or more target genes comprises a UHRF Family member. In some embodiments, the one or more target genes comprises a Vitamin A Family member. In some embodiments, the one or more target genes comprises an Interleukin Family member. In some embodiments, the one or more target genes comprises a KLK Family member. In some embodiments, the one or more target genes comprises a KRT Family member. In some embodiments, the one or more target genes comprises a MUCL Family member. In some embodiments, the one or more target genes comprises a PDCD Family member. In some embodiments, the one or more target genes comprises a SPRR Family member. Non-limiting examples of gene families and gene family members that may be used as target genes are included in Table 1. Some embodiments include a combination of the one or more target genes.

TABLE 1

| | | Non-Limiting |
|---|---|---|
| | | Example |
| | | of Gene |
| Gene Family Name | Full Name of the Family | Family Member |
| ADAMTSL Family | ADAMTS like Family | ADAMTSL4 |
| CDKN Family | Cyclin Dependent Kinase | CDKN1A |
| | Inhibitor (CDKN) Family | |
| CST Family | Cystatin (CST) Family | CST6 |
| KIF Family | kinesin family member | KIF18B |
| MKI Family | marker of proliferation | MKI67 |
| | Ki-67 | |
| SLAM Family | SLAM family member | SLAM F7 |
| TRIP Family | thyroid hormone receptor | TRI P13 |
| | interactor | |
| UHRF Family | ubiquitin like with PHD | UHRF1 |
| | and ring finger domains | |
| Vitamin A Family | Vitamin A Family | CRABP2 |
| Interleukin Family (1) | Interleukin Family (1) | IL1RN |
| Interleukin Family (2) | Interleukin Family (2) | IL22RA1 |
| Interleukin Family (3) | Interleukin Family (3) | IL36B |
| Interleukin Family (4) | Interleukin Family (4) | IL36G |
| KLK Family | Kallikrein (KLK) Family | KLK10 |
| KRT Family | Keratin (KRT) Family | KRT17 |
| MUCL Family | Mucin-Like (MUCL) | MUCL1 |
| | Protein Family | |
| PDCD Family | Programmed Cell Death | PDCD4 |
| | (PDCD) Protein Family | |
| SPRR Family | Small Proline-Rich Protein | SPRR1A |
| | (SPRR) Family | |

In some embodiments, the one or more target genes comprises ADAMTSL4, CDKN1A, CDKN2A, CST6, KIF18B, MKI67, SLAMF7, TRIP13, UHRF1, CRABP2, IL1RN, IL22RA1, IL36B, IL36G, KLK1, KRT17, MUCL1, PDCD4, or SPRR1A. Some embodiments include 1 target gene. Some embodiments include multiple target genes. In some embodiments, one or more target genes include a combination of ADAMTSL4, CDKN1A, CDKN2A, CST6, KF18B, MKI67, SLAMF7, TRIP13, UHRF1, CRABP2, IL1RN, IL22RA1, IL36B, IL36G, KLK10, KRT17, MUCL1, PDCD4, and/or SPRR1A. In some embodiments, the one or more target genes comprise the gene families of any one or more of these genes. In some embodiments, the one or more target genes comprises CDKN1A, CST6, CRABP2, IL1RN, IL22RA1, KLK1, MUCL1, PDCD4, or SPRR1A, or a combination thereof. In some embodiments, the one or more target genes comprises CDKN1A, CST6, CRABP2, IL1RN, IL22RA1, KLK10, MUCL1, PDCD4, and SPRR1A. In some embodiments, the one or more target genes comprises CST6, CRABP2, IL1RN, IL36G, MUCL1, PDCD4, or SPRR1A, or a combination thereof. In some embodiments, the one or more target genes comprises CST6, CRABP2, IL1RN, IL36G, MUCL1, PDCD4, and SPRR1A. In some embodiments, the one or more target genes comprises CST6, SPRR1A, MUCL1, or PDCD4, or a combination thereof. In some embodiments, the one or more target genes comprises CST6, SPRR1A, MUCL1, and PDCD4. In some embodiments, the one or more target genes comprises CRABP2, IL36G, or IL1RN, or a combination thereof. In some embodiments, the one or more target genes comprises CRABP2, IL36G, and IL1RN. In some embodiments, the one or more target genes comprise MUCL1, PDCD4, CST6, SPRR1A, IL1RN, CRABP2, or IL36G, or a combination thereof. In some embodiments, the one or more target genes comprise MUCL1, PDCD4, CST6, SPRR1A, IL1RN, CRABP2, and IL36G. In some embodiments, the one or more target genes comprises CDKN1A, CST6, CRABP2, IL1RN, IL36G, KLK10, MUCL1, PDCD4, or SPRR1A, or a combination thereof. In some embodiments, the one or more target genes comprises CDKN1A, CST6, CRABP2, IL1RN, IL36G, KLK10, MUCL1, PDCD4, and SPRR1A. In some embodiments, the one or more target genes comprises CST6, SPRR1A, MUCL1, KLK10, SPRR1A, or PDCD4, or a combination thereof. In some embodiments, the one or more target genes comprises CST6, SPRR1A, MUCL1, KLK10, SPRR1A, and PDCD4. In some embodiments, the one or more target genes comprise one or more genes in the gene families of any one or more of these genes or combinations of genes.

In some embodiments, the one or more target genes are genes linked to UV damage. In some embodiments, the one or more target genes include one or more retinoid response genes. In some embodiments, the one or more target genes include one or more genes linked to hydration. An example of a gene linked to hydration includes but is not limited to aquaporin. In some embodiments, disclosed herein is a method of detecting the expression level of a gene family or a gene family member, which is associated with UV exposure (e.g., from sun damage) of the skin of a subject. In some instances, the method comprises measuring or detecting the expression level of a Vitamin A gene family or family member, a Programmed Cell Death Protein gene family or family member, a Small Proline Rich Protein gene family or family member, an Interleukin 1/2 gene family or family member, a cystatin gene family or family member, or a combination thereof.

In some embodiments, the target gene encodes a microRNA. In some embodiments, the microRNA is a small non-coding RNA. In some embodiments, the microRNA comprises or consists of 19-25 nucleotides. In some embodiments, the microRNA is from an intronic, intergenic, or antisense nucleic acid region. In some embodiments, the microRNA regulates post-transcriptional gene expression. Some embodiments described herein, include an RNA comprising a microRNA as described herein. Measuring or determining expression levels of one or more microRNAs may be useful because some microRNAs are dysregulated in a skin damage such as UV skin damage. In some embodiments, an amount of the microRNA is increased in UV skin damage relative to a non-UV skin damage control. In some embodiments, an amount of the microRNA is decreased in UV skin damage relative to a non-UV skin damage control. In some embodiments, the microRNA expression is measured by microarray followed by PCR analysis.

In some embodiments, disclosed herein is a method of detecting the expression level of a gene from a gene classifier, which is associated with UV exposure (e.g., sun damage) of the skin of a subject. In some instances, the method comprises detecting the expression level of cellular retinoic acid binding protein 2 (CRABP2), interleukin 1 receptor antagonist (IL1RN), interleukin-36 gamma (IL36G), small breast epithelial mucin (MUCL1), programmed cell death 4 (PDCD4), small proline-rich protein 1A (SPRR1A), cystatin E/M (CST6), kallikrein related peptidase 10 (KLK10), or a combination thereof. In some instances, the method comprises (a) isolating nucleic acids from a skin sample obtained from the subject, wherein the skin sample (e.g., comprising cells from the stratum corneum); and (b) detecting the expression level of CRABP2, IL1RN, IL36G, MUCL1, PDCD4, SPRR1A, CST6, KLK10, or a combination thereof, by contacting the isolated nucleic acids with a set of probes that recognizes CRABP2, IL1RN, IL36G, MUCL1, PDCD4, SPRR1A, CST6, KLK10, or a combination thereof, and detects binding between CRABP2, IL1RN, IL36G, MUCL1, PDCD4, SPRR1A, CST6, KLK10, or a combination thereof and the set of probes.

In some embodiments, the method comprises detecting the expression levels of two or more, three or more, or four or more of genes from the gene classifier: CRABP2, IL1RN, IL36G, MUCL1, PDCD4, SPRR1A, CST6, and KLK10. In some cases, the method comprises detecting the expression levels of SPRR1A and MUCL1. In some cases, the method comprises detecting the expression levels of CRABP2, IL36G, MUCL1, PDCD4, and CST6. In some cases, the method comprises detecting the expression levels of IL1RN, SPRR1A, and KLK10. In some cases, the method comprises detecting the expression levels of IL1RN and IL36G. In some cases, the method comprises detecting the expression levels of CRABP2, MUCL1, PDCD4, SPRR1A, CST6, and KLK10. In some cases, the method comprises detecting the expression levels of CRABP2, IL1RN, IL36G, MUCL1, PDCD4, SPRR1A, CST6, and KLK10.

In some instances, the expression level is a downregulated gene expression level. In some instances, the expression level is a down-regulated gene expression level, compared to a gene expression level of an equivalent gene from a control sample. In some cases, the control sample is a normal skin sample. In some cases, the gene expression level of CRABP2, MUCL1, PDCD4, SPRR1A, CST6, KLK10, or a combination thereof is down-regulated. In some instances, the down-regulated gene expression level occurs within 24 hours from time of UV exposure (e.g., sun exposure or sun damage).

In some instances, the gene expression level of CRABP2, MUCL1, PDCD4, SPRR1A, CST6, or KLK10 is decreased by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the gene expression level of CRABP2, MUCL1, PDCD4, SPRR1A, CST6, or KLK10 is decreased by at least 10-fold. In some cases, the gene expression level of CRABP2, MUCL1, PDCD4, SPRR1A, CST6, or KLK10 is decreased by at least 20-fold. In some cases, the gene expression level of CRABP2, MUCL1, PDCD4, SPRR1A, CST6, or KLK10 is decreased by at least 30-fold. In some cases, the gene expression level of CRABP2, MUCL1, PDCD4, SPRR1A, CST6, or KLK10 is decreased by at least 40-fold. In some cases, the gene expression level of CRABP2, MUCL1, PDCD4, SPRR1A, CST6, or KLK0 is decreased by at least 50-fold. In some cases, the gene expression level of CRABP2, MUCL1, PDCD4, SPRR1A, CST6, or KLK10 is decreased by at least 80-fold. In some cases, the gene expression level of CRABP2, MUCL1, PDCD4, SPRR1A, CST6, or KLK10 is decreased by at least 100-fold. In some cases, the gene expression level of CRABP2, MUCL1, PDCD4, SPRR1A, CST6, or KLK10 is decreased by at least 130-fold. In some cases, the gene expression level of CRABP2, MUCL1, PDCD4, SPRR1A, CST6, or KLK10 is decreased by at least 150-fold. In some cases, the gene expression level of CRABP2, MUCL1, PDCD4, SPRR1A, CST6, or KLK10 is decreased by at least 200-fold. In some cases, the gene expression level of CRABP2, MUCL1, PDCD4, SPRR1A, CST6, or KLK10 is decreased by at least 300-fold. In some cases, the gene expression level of CRABP2, MUCL1, PDCD4, SPRR1A, CST6, or KLK10 is decreased by at least 500-fold. In some cases, the decreased gene expression level is compared to a gene expression level of an equivalent gene from a control sample. In some cases, the control sample is a normal skin sample. In some instances, the down-regulated gene expression level occurs within 24 hours from time of UV exposure (e.g., sun exposure or sun damage).

In some cases, the gene expression level of CRABP2, MUCL1, PDCD4, SPRR1A, CST6, or KLK10 is decreased by at least 10%, 20%, 30%, 40%, 50%, 60%, 70%, 80%, 90%, 100%, 200%, 300%, 400%, 500%, or more. In some cases, the gene expression level of CRABP2, MUCL1, PDCD4, SPRR1A, CST6, or KLK10 is decreased by at least 10%. In some cases, the gene expression level of CRABP2, MUCL1, PDCD4, SPRR1A, CST6, or KLK10 is decreased by at least 20%. In some cases, the gene expression level of CRABP2, MUCL1, PDCD4, SPRR1A, CST6, or KLK10 is decreased by at least 30%. In some cases, the gene expression level of CRABP2, MUCL1, PDCD4, SPRR1A, CST6, or KLK10 is decreased by at least 40%. In some cases, the gene expression level of CRABP2, MUCL1, PDCD4, SPRR1A, CST6, or KLK10 is decreased by at least 50%. In some cases, the gene expression level of CRABP2, MUCL1, PDCD4, SPRR1A, CST6, or KLK10 is decreased by at least 80%. In some cases, the gene expression level of CRABP2, MUCL1, PDCD4, SPRR1A, CST6, or KLK10 is decreased by at least 90%. In some cases, the gene expression level of CRABP2, MUCL1, PDCD4, SPRR1A, CST6, or KLK10 is decreased by at least 100%. In some cases, the gene expression level of CRABP2, MUCL1, PDCD4, SPRR1A, CST6, or KLK10 is decreased by at least 150%. In some cases, the gene expression level of CRABP2, MUCL1, PDCD4, SPRR1A, CST6, or KLK10 is decreased by at least 200%. In some cases, the gene expression level of CRABP2, MUCL1, PDCD4, SPRR1A, CST6, or KLK10 is decreased by at least 300%. In some cases, the gene expression level of CRABP2, MUCL1, PDCD4, SPRR1A, CST6, or KLK10 is decreased by at least 500%. In some cases, the decreased gene expression level is compared to a gene expression level of an equivalent gene from a control sample. In some cases, the control sample is a normal skin sample. In some instances, the down-regulated gene expression level occurs within 24 hours from time of UV exposure (e.g., sun exposure or sun damage).

In some instances, the expression level is an up-regulated gene expression level. In some cases, the gene expression level of IL1RN or IL36G is up-regulated. In some cases, the up-regulated gene expression level is compared to a gene expression level of an equivalent gene from a control sample. In some cases, the control sample is a normal skin sample. In some instances, the up-regulated gene expression level occurs within 24 hours from time of UV exposure (e.g., sun exposure or sun damage).

In some instances, the gene expression level of IL1RN or IL36G is up-regulated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some cases, the gene expression level of IL1RN or IL36G is up-regulated by at least 1-fold. In some cases, the gene expression level of IL1RN or IL36G is up-regulated by at least 5-fold. In some cases, the gene expression level of IL1RN or IL36G is up-regulated by at least 10-fold. In some cases, the gene expression level of IL1RN or IL36G is up-regulated by at least 20-fold. In some cases, the gene expression level of IL1RN or IL36G is up-regulated by at least 30-fold. In some cases, the gene expression level of IL1RN or IL36G is up-regulated by at least 40-fold. In some cases, the gene expression level of IL1RN or IL36G is up-regulated by at least 50-fold. In some cases, the gene expression level of IL1RN or IL36G is up-regulated by at least 80-fold. In some cases, the gene expression level of IL1RN or IL36G is up-regulated by at least 100-fold. In some cases, the gene expression level of IL1RN or IL36G is up-regulated by at least 200-fold. In some cases, the up-regulated gene expression level is compared to a gene expression level of an equivalent gene from a control sample. In some cases, the control sample is a normal skin sample. In some instances, the up-regulated gene expression level occurs within 24 hours from time of UV exposure (e.g., sun exposure or sun damage).

In some instances, the gene expression level of IL1RN or IL36G is up-regulated by at least 10%, 20%, 30%, 40%, 50%, 60%, 70%, 80%, 90%, 100%, 200%, 300%, 400%, 500%, or more. In some cases, the gene expression level of IL1RN or IL36G is up-regulated by at least 10%. In some cases, the gene expression level of IL1RN or IL36G is up-regulated by at least 20%. In some cases, the gene expression level of IL1RN or IL36G is up-regulated by at least 30%. In some cases, the gene expression level of IL1RN or IL36G is up-regulated by at least 40%. In some cases, the gene expression level of IL1RN or IL36G is up-regulated by at least 50%. In some cases, the gene expression level of IL1RN or IL36G is up-regulated by at least 80%. In some cases, the gene expression level of IL1RN or IL36G is up-regulated by at least 100%. In some cases, the gene expression level of IL1RN or IL36G is up-regulated by at least 200%. In some cases, the up-regulated gene expression level is compared to a gene expression level of an equivalent gene from a control sample. In some cases, the control sample is a normal skin sample. In some instances, the up-regulated gene expression level occurs within 24 hours from time of UV exposure (e.g., sun exposure or sun damage).

In some embodiments, the set of probes recognizes at least one but no more than eight genes selected from CRABP2, IL1RN, IL36G, MUCL1, PDCD4, SPRR1A, CST6, and KLK10. In some cases, the set of probes recognizes SPRR1A and MUCL1. In some cases, the set of probes recognizes CRABP2, IL36G, MUCL1, PDCD4, and CST6. In some cases, the set of probes recognizes IL1RN, SPRR1A, and KLK10. In some cases, the set of probes recognizes IL1RN and IL36G. In some cases, the set of probes recognizes CRABP2, MUCL1, PDCD4, SPRR1A, CST6, and KLK10. In some cases, the set of probes recognizes CRABP2, IL1RN, IL36G, MUCL1, PDCD4, SPRR1A, CST6, and KLK10.

In some embodiments, the method further comprises detecting the expression levels of interleukin 22 receptor subunit alpha 1 (IL22RA1), interleukin 36 Beta (IL36B), keratin 17 (KRT17), a disintegrin and metalloproteinase with thrombospondin motifs-like 4 (ADAMTSL4), cyclin dependent kinase inhibitor 1A (CDKN1A), kinesin family member 18B (KIF18B), marker of proliferation Ki-67 (MKI67), SLAM family member 7 (SLAMF7), thyroid hormone receptor interactor 13 (TRIP13), ubiquitin like with PHD and ring finger domains 1 (UHRF1), or a combination thereof. In some cases, the detecting comprises contacting the isolated nucleic acids with an additional set of probes that recognizes IL22RA1, IL36B, KRT7, ADAMTSL4, CDKN1A, KIF18B, MKI67, SLAMF7, TRIP13, UHRF1, or a combination thereof, and detects binding between IL22RA1, IL36B, KRT17, ADAMTSL4, CDK1A, KIF8B, MKI67, SLAMF7, TRIP13, UHRF1, or a combination thereof and the additional set of probes.

In some cases, the additional set of probes recognizes one but no more than ten genes. In some cases, the additional set of probes recognizes 2, 3, 4, 5, 6, 7, 8, 9, or 10 genes selected from IL22RA1, IL36B, KRT17, ADAMTSL4, CDK1A, KIF8B, MKI67, SLAMF7, TRIP13, and UHRF1.

In some cases, the expression level of one or more genes selected from IL22RA1, IL36B, KRT17, ADAMTSL4, CDK1A, KIF18B, MKI67, SLAMF7, TRIP13, and UHRF1 is an elevated gene expression level. In such cases, the gene expression level is elevated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some instances, the gene expression level is elevated by at least 10%, 20%, 30%, 40%, 50%, 60%, 70%, 80%, 90%, 100%, 200%, 300%, 400%, 500%, or more. In some instances, the expression level is compared to a gene expression level of an equivalent gene from a control sample. In some instances, the control sample is a normal skin sample.

In additional cases, the expression level of one or more genes selected from IL22RA1, IL36B, KRT17, ADAMTSL4, CDK1A, KIF18B, MKI67, SLAMF7, TRIP13, and UHRF1 is a down-regulated gene expression level. In such cases, the gene expression level is down-regulated by at least 1-fold, 2-fold, 3-fold, 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 150-fold, 200-fold, 300-fold, 500-fold, or more. In some instances, the gene expression level is down-regulated by at least 10%, 20%, 30%, 40%, 50%, 60%, 70%, 80%, 90%, 100%, 200%, 300%, 400%, 500%, or more. In some instances, the expression level is compared to a gene expression level of an equivalent gene from a control sample. In some instances, the control sample is a normal skin sample.

In some embodiments, a method described herein further comprises detecting a skin region that is damaged by UV (e.g., sun damage). In some cases, also described herein include a method monitoring the skin region that has been damaged by UV, for about 1 week, 2 weeks, 3 weeks, 1 month, 2 months, 6 months, or more.

In some instances, the method has an improved specificity, of at least or about 70%, 75%, 80%, 85%, 90%, or more than 95% when detecting the gene expression level of CRABP2, IL1RN, IL36G, MUCL1, PDCD4, SPRR1A, CST6, KLK10, or a combination thereof. In some embodiments, the specificity is at least or about 70%, 75%, 80%, 85%, 90%, or more than 95% when detecting the gene expression level of IL22RA1, IL36B, KRT7, ADAMTSL4, CDKN1A, KIF8B, MKI67, SLAMF7, TRIP13, UHRF1, or a combination thereof.

In some cases, the method also has an improved sensitivity. In some embodiments, the sensitivity is at least or about 70%, 75%, 80%, 85%, 90%, or more than 95% when detecting the gene expression levels of CRABP2, IL1RN, IL36G, MUCL1, PDCD4, SPRR1A, CST6, KLK10, or a combination thereof. In some cases, the sensitivity is at least or about 70%, 75%, 80%, 85%, 90%, or more than 95% when detecting the gene expression levels of IL22RA1, IL36B, KRT17, ADAMTSL4, CDKN1A, KIF18B, MKI67, SLAMF7, TRIP13, UHRF1, or a combination thereof.

In some embodiments, a method described herein comprises detecting gene expression levels from a first gene classifier and a second gene classifier in a subject in need thereof, comprising: (a) isolating nucleic acids from a skin sample obtained from the subject, wherein the skin sample (e.g., comprising cells from the stratum corneum); (b) detecting the expression levels of one or more genes from the first gene classifier: CRABP2, IL1RN, IL36G, MUCL1, PDCD4, SPRR1A, CST6, and KLK1, by contacting the isolated nucleic acids with a set of probes that recognizes one or more genes from the first gene classifier, and detects binding between one or more genes from the first gene classifier and the set of probes; and (c) detecting the expression levels of one or more genes from the second gene classifier: IL22RA1, IL36B, KRT17, ADAMTSL4, CDKN1A, KIF18B, MKI67, SLAMF7, TRIP13, and UHRF1, by contacting the isolated nucleic acids with an additional set of probes that recognizes one or more genes from the second gene classifier, and detects binding between one or more genes from the second gene classifier and the additional set of probes.

In some embodiments, a number of probes in the set of probes described above is at least or about 1, 2, 3, 5, 6, 7, 8, 9, 10, 11, 12, 13, 14, 15, 16, 17, 18, 19, 20, 25, 30, or more than 30 probes. In some embodiments, the number of probes in the set of probes is about 6 probes. In some embodiments, the number of probes in the set of probes is about 7 probes. In some embodiments, the number of probes in the set of probes is about 8 probes. In some embodiments, the number of probes in the set of probes is about 9 probes. In some embodiments, the number of probes in the set of probes is about 13 probes.

In some embodiments, the set of probes comprises one or more primer pairs. In some embodiments, a number of primer pairs is at least or about 1, 2, 3, 5, 6, 7, 8, 9, 10, 11, 12, 13, 14, 15, 16, 17, 18, 19, 20, 25, 30, or more than 30 primer pairs. In some embodiments, the number of primer pairs is about 8 primer pairs. In some embodiments, the number of primer pairs is about 9 primer pairs. In some embodiments, the number of primer pairs is about 10 primer pairs. In some embodiments, the number of primer pairs is about 6 primer pairs. In some embodiments, the number of primer pairs is about 7 primer pairs. In some embodiments, the number of primer pairs is about 13 primer pairs.

In some embodiments, one or more probes in the set of probes is labeled. In some embodiments, the one or more probe is labeled with a radioactive label, a fluorescent label, an enzyme, a chemiluminescent tag, a colorimetric tag, an affinity tag or other labels or tags that are known in the art.

Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione.

In some embodiments, the fluorescent label is a fluorophore, a fluorescent protein, a fluorescent peptide, quantum dots, a fluorescent dye, a fluorescent material, or variations or combinations thereof.

Exemplary fluorophores include, but are not limited to, Alexa-Fluor dyes (e.g., Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750), APC, Cascade Blue, Cascade Yellow and R-phycoerythrin (PE), DyLight 405, DyLight 488, DyLight 550, DyLight 650, DyLight 680, DyLight 755, DyLight 800, FITC, Pacific Blue, PerCP, Rhodamine, and Texas Red, Cy5, Cy5.5, Cy7.

Examples of fluorescent peptides include GFP (Green Fluorescent Protein) or derivatives of GFP (e.g., EBFP, EBFP2, Azurite, mKalama1, ECFP, Cerulean, CyPet, YFP, Citrine, Venus, and YPet.

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earthmetal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, and rhodamine 101 sulfonyl chloride (sold under the tradename of TEXAS RED®). In some embodiments, the cyanine dye is Cy3, Cy3B, Cy3.5, Cy5, Cy5.5, Cy7, IRDYE680, Alexa Fluor 750, IRDye800CW, or ICG.

In some embodiments, the gene expression levels of CRABP2, IL1RN, IL36G, MUCL1, PDCD4, SPRR1A, CST6, KLK10, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the gene expression levels of IL22RA1, IL36B, KRT17, ADAMTSL4, CDKN1A, KIF18B, MKI67, SLAMF7, TRIP13, UHRF1, or a combination thereof is measured using PCR. Examples of PCR techniques include, but are not limited to quantitative PCR (qPCR), single cell PCR, PCR-RFLP, digital PCR (dPCR), droplet digital PCR (ddPCR), single marker qPCR, hot start PCR, and Nested PCR.

In some embodiments, the expression levels are measured using qPCR. In some embodiments, the qPCR comprises use of fluorescent dyes or fluorescent probes. In some embodiments, the fluorescent dye is an intercalating dye. Examples of intercalating dyes include, but are not limited to, intercalating dyes include SYBR green I, SYBR green II, SYBR gold, ethidium bromide, methylene blue, Pyronin Y, DAPI, acridine orange, Blue View, or phycoerythrin. In some embodiments, the qPCR comprises use of more than one fluorescent probe. In some embodiments, the use of more than one fluorescent probes allows for multiplexing. For example, different non-classical variants are hybridized to different fluorescent probes and can be detected in a single qPCR reaction. Some embodiments include detecting or measuring an amount of binding between genes of interest and a set of probes, and includes detecting or measuring a fluorescent dye or a fluorescent probe.

Disclosed herein, in some embodiments, are methods of detecting, assessing, measuring, or determining the presence of a skin damage such as UV skin damage. Some embodiments include isolating nucleic acids from a skin sample obtained from a subject. Some embodiments include measuring, detecting, receiving, or using an expression level of a target gene. Some embodiments include detecting an expression level of a target gene in the skin sample. Some embodiments include measuring an expression level of a target gene in the skin sample. Some embodiments include receiving an expression level of a target gene in the skin sample. Some embodiments include using an expression level of a target gene in the skin sample. Some embodiments include measuring an expression level of a target gene in the skin sample. Some embodiments include measuring or detecting an expression level of the target gene.

Some embodiments include multiple target genes. For example, multiple target genes may be measured, detected, or used in the methods described herein. Some embodiments include determining the presence of UV skin damage based on a presence or expression level of multiple target genes. Some embodiments include determining the presence of UV skin damage based on a presence or expression level of a first target gene, and based on a presence or expression level of a second target gene.

Some embodiments include more than one target gene (e.g., at least one target gene). For example, the method may include measuring, detecting, receiving, or using expression levels of multiple target genes. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more target genes. Some embodiments include 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 250, 300, 350, 400, 450, 500, 550, 600, 650, 700, 750, 800, 850, 900, 950, 1000, or more target genes, or a range of target genes defined by any two of the aforementioned integers. For example, some embodiments include measuring or detecting an expression level of 17 target genes. Some embodiments include measuring or detecting an expression level of 10 target genes. Some embodiments include measuring or detecting an expression level of 9 target genes. Some embodiments include measuring or detecting an expression level of 8 target genes. Some embodiments include measuring or detecting an expression level of 7 target genes. Some embodiments include measuring or detecting an expression level of 6 target genes. Some embodiments include measuring or detecting an expression level of 5 target genes. Some embodiments include measuring or detecting an expression level of 4 target genes. Some embodiments include measuring or detecting an expression level of 3 target genes. Some embodiments include measuring or detecting an expression level of 2 target genes. Some embodiments include measuring or detecting an expression level of 1 target gene. Some embodiments include measuring or detecting an expression level of 1-4 target genes. Some embodiments include measuring or detecting an expression level of 1-7 target genes. Some embodiments include measuring or detecting an expression level of 1-10 target genes. Some embodiments include measuring or detecting an expression level of 1-100 target genes. Some embodiments include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, or at least 100 target genes. Some embodiments include no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95, or no more than 100 target genes.

In some embodiments, the nucleic acids comprise RNA. In some embodiments, the nucleic acids comprise mRNA. In some embodiments, measuring or detecting the expression level of the target gene comprises measuring or detecting an amount of RNA or mRNA encoded by a nucleic acid comprising the target gene. In some embodiments, measuring or detecting the expression level of the target gene comprises measuring or detecting an amount of mRNA encoded by a nucleic acid comprising the target gene. In some embodiments, using or receiving the expression level of the target gene comprises using or receiving information on an amount of RNA or mRNA encoded by a nucleic acid comprising the target gene.

Disclosed herein, in some embodiments, are methods of determining the presence or amount of UV skin damage, comprising isolating nucleic acids from a skin sample obtained from a subject, and detecting an expression level of a target gene. Some embodiments include measuring or detecting an expression level of the target gene. Some embodiments include detecting an expression level of the target gene. Some embodiments include measuring an expression level of the target gene. Some embodiments include more than one target gene (e.g., at least one target gene). In some embodiments, measuring or detecting the expression level of the target gene comprises measuring or detecting an amount of RNA or mRNA encoded by a nucleic acid comprising the target gene.

Disclosed herein, in some embodiments, are methods of determining the presence of UV skin damage in a skin sample. Some embodiments include identifying a subject suspected of having UV skin damage. Some embodiments include isolating nucleic acids from a skin sample obtained from the subject. In some embodiments, the skin sample is obtained by applying an adhesive patch to a skin region of the subject. In some embodiments, the adhesive patch is applied in a manner sufficient to adhere skin sample cells to the adhesive patch. In some embodiments, the skin sample is further obtained by removing the adhesive patch from the skin sample in a manner sufficient to retain the adhered skin sample cells to the adhesive patch. In some embodiments, the skin sample cells comprise cells from the stratum corneum. In some embodiments, the skin sample cells consist of cells from the stratum corneum. Some embodiments include isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise or consist of cells from the stratum corneum. Some embodiments include measuring or detecting an expression level of at least one target gene. In some embodiments, the at least one target gene is known to be upregulated or downregulated in subjects with UV skin damage. Some embodiments include contacting the isolated nucleic acids with a set of probes that recognize the target gene. Some embodiments include detecting binding between the at least one target gene and the set of probes.

Disclosed herein, in some embodiments, are methods of determining the presence of UV skin damage in a skin sample, comprising: identifying a subject suspected of having UV skin damage; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and measuring or detecting an expression level of at least one target gene known to be upregulated or downregulated in subjects with UV skin damage, by contacting the isolated nucleic acids with a set of probes that recognize the target gene, and detecting binding between the at least one target gene and the set of probes.

Disclosed herein, in some embodiments, are methods for non-invasively identifying UV skin damage in a subject suspected of having the UV skin damage. In some embodiments, the method includes isolating nucleic acids from a skin sample adhered to an adhesive patch, the skin sample having been obtained from the subject suspected of having the UV skin damage. Some embodiments include contacting the isolated nucleic acids with a set of probes that recognize one or more genes of interest implicated in the UV skin damage. Some embodiments include detecting or measuring an amount of binding between the genes of interest and the set of probes. Some embodiments include comparing the amount of binding between the genes of interest and the set of probes to a control or threshold amount of binding. Some embodiments include identifying the subject as having the UV skin damage, or as not having the UV skin damage, based on the amount of binding between the genes of interest and the set of probes relative to the control or threshold of binding. In some embodiments, identifying the subject as having the UV skin damage, or as not having the UV skin damage, based on the amount of binding between the genes of interest and the set of probes relative to the control or threshold amount of binding comprises applying the amount of binding to a random forest model, a boosting model, a logit model, a lasso model, or a combination thereof, and comprises taking into account interactions of the genes of interest. Some embodiments include administering an effective amount of a therapeutic agent to the subject identified as having the UV skin damage.

Disclosed herein, in some embodiments, are methods for non-invasively identifying UV skin damage in a subject suspected of having UV skin damage, the method comprising: isolating nucleic acids from a skin sample adhered to an adhesive patch, the skin sample having been obtained from the subject suspected of having the UV skin damage; contacting the isolated nucleic acids with a set of probes that recognize one or more genes of interest implicated in UV skin damage; and detecting or measuring an amount of binding between the genes of interest and the set of probes.

Disclosed herein, in some embodiments, are methods for non-invasively identifying UV skin damage. Some embodiments include identifying a subject suspected of having the UV skin damage. Some embodiments include applying an adhesive patch to the subject's skin in a manner sufficient to adhere a skin sample to the adhesive patch. Some embodiments include removing the adhesive patch from the subject's skin in a manner sufficient to retain the skin sample adhered to the adhesive patch. Some embodiments include obtaining expression levels of genes of interest implicated in UV skin damage, or determining an amount of binding between the genes of interest and a set of probes that recognize the genes of interest.

Disclosed herein, in some embodiments, are methods for non-invasively identifying UV skin damage, comprising: identifying a subject suspected of having the UV skin damage; applying an adhesive patch to the subject's skin in a manner sufficient to adhere a skin sample to the adhesive patch; removing the adhesive patch from the subject's skin in a manner sufficient to retain the skin sample adhered to the adhesive patch; and obtaining expression levels of genes of interest implicated in UV skin damage, or determining an amount of binding between the genes of interest and a set of probes that recognize the genes of interest.

Disclosed herein, in some embodiments, are methods for non-invasively identifying UV skin damage in a subject suspected of having UV skin damage. In some embodiments, the method includes isolating nucleic acids from a skin sample adhered to an adhesive patch. In some embodiments, the skin sample was obtained from the stratum corneum of the subject suspected of having UV skin damage. Some embodiments include contacting the isolated nucleic acids with a set of probes that recognize target genes; and detecting or measuring an amount of binding between the nucleic acids and the set of probes.

Disclosed herein, in some embodiments, are methods for non-invasively identifying UV skin damage in a subject suspected of having UV skin damage, the method comprising: isolating nucleic acids from a skin sample adhered to an adhesive patch, the skin sample having been obtained from the stratum corneum of the subject suspected of having UV skin damage; contacting the isolated nucleic acids with a set of probes that recognize target genes; and detecting or measuring an amount of binding between the nucleic acids and the set of probes.

Disclosed herein, in some embodiments, are methods for non-invasively identifying UV skin damage. In some embodiments, the method includes identifying a subject suspected of having UV skin damage. Some embodiments include applying an adhesive patch to the subject's skin in a manner sufficient to adhere a skin sample to the adhesive patch. Some embodiments include removing the adhesive patch from the subject's skin in a manner sufficient to retain the skin sample adhered to the adhesive patch. Some embodiments include obtaining expression levels of target genes implicated in UV skin damage. Some embodiments include determining an amount of binding between the genes of interest and a set of probes that recognize the target genes.

Disclosed herein, in some embodiments, are methods for non-invasively identifying UV skin damage, comprising: identifying a subject suspected of having UV skin damage; applying an adhesive patch to the subject's skin in a manner sufficient to adhere a skin sample to the adhesive patch; removing the adhesive patch from the subject's skin in a manner sufficient to retain the skin sample adhered to the adhesive patch; and obtaining expression levels of target genes implicated in UV skin damage, or determining an amount of binding between the genes of interest and a set of probes that recognize the target genes.

Disclosed herein, in certain embodiments, is a method of assessing ultraviolet (UV) skin damage. Some embodiments include identifying a subject exposed to UV radiation. Some embodiments include isolating nucleic acids from a skin sample obtained from the subject. In some embodiments, the skin sample is obtained by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch. In some embodiments, the skin sample cells comprise cells from the stratum corneum. Some embodiments include measuring or detecting an expression level of at least one target gene known to be upregulated or downregulated in subjects with UV skin damage. Some embodiments include contacting the isolated nucleic acids with a set of probes that recognize the target gene. Some embodiments include measuring or detecting binding between the at least one target gene and the set of probes.

Disclosed herein, in certain embodiments, is a method of treating a subject with ultraviolet (UV) skin damage. Some embodiments include identifying a subject exposed to UV radiation. Some embodiments include isolating nucleic acids from a skin sample obtained from the subject. In some embodiments, the skin sample is obtained by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch. In some embodiments, the skin sample cells comprise cells from the stratum corneum. Some embodiments include measuring or detecting an expression level of at least one target gene known to be upregulated or downregulated in subjects with UV skin damage. Some embodiments include contacting the isolated nucleic acids with a set of probes that recognize the target gene. Some embodiments include measuring or detecting binding between the at least one target gene and the set of probes. Some embodiments include determining whether the subject has UV skin damage based on the expression level of the at least one target gene. Some embodiments include administering a skin treatment to the subject when the subject is determined to have UV skin damage based on the expression level of the at least one target gene. Some embodiments include not administering the skin treatment to the subject when the subject is not determined to have UV skin damage based on the expression level of the at least one target gene. In some embodiments, the determination of whether the subject has UV skin damage is based on comparing the expression level(s) of the at least one target gene to a threshold amount of expression.

Disclosed herein, in some embodiments, are methods of assessing UV skin damage. Some uses of such a method include but are not limited to testing of sunscreen products, testing of UV exposure supplements, and monitoring sun exposure. In some embodiments, the sun exposure monitoring is performed on a patient under the supervision of a medical professional. In some embodiments, the sun exposure monitoring is incorporated into a consumer product. In some embodiments, the sun exposure monitoring is self directed by consumers, and in some instances is not performed under a medical professional's supervision.

Some embodiments relate to a UV skin damage assessment comprising a method as described herein. For example, the UV skin damage assessment may include the measurement of one or more target genes, and/or generating a UV exposure score. The UV skin damage assessment may be initiated by consumers, cosmetologists or clinicians depending on the nature of the UV damage (e.g. UV damage related accelerated aging, testing, or recommendations of anti-aging products including sunscreens with or without repair enzymes). The UV skin damage assessment may be initiated based on the presence of physical evidence of UV skin damage such as sun damaged skin, wrinkles, pigment changes, loss of elastosis, or emerging lesions related to UV damage (e.g. actinic keratoses).

In some embodiments, the UV skin damage assessment is performed or initiated by a medical professional on a subject. In some cases, a clinician would be assessing a patient and determining if the a UV skin damage assessment. In some embodiments, the UV skin damage assessment includes a determination of UV skin damage based on the subject's medical history (e.g. actinic keratoses, a skin cancer such as melanoma, SCC or BCC, and/or solar lentigo). In some cases, the clinician gets a report of high risk patients. In some cases, a patient file is flagged for a UV skin damage assessment based on medical history. In some embodiments, the clinician orders the test yearly, or more often depending on subjects.

In some embodiments, the UV skin damage assessment is performed or initiated by the subject. For example, the UV skin damage assessment may be an annual screening test sent to the patient, or that the patient initiates and sends to a diagnostic lab or to a clinician. For example, the subject may receive skin sampling patches that the subject uses to collect his or her own skin samples, and sends to the laboratory or clinician. In some embodiments, the patient is sent a kit, on an annual basis for example, after having been identified by a medical record, algorithm or clinician. In some embodiments, the patient is simply concerned and orders the test.

In some embodiments, the need for a UV skin damage assessment is determined by a computer or algorithm. In some embodiments, photography or images are used to demonstrate sun damage, and a need for the subject to have a UV skin damage assessment. Some embodiments include a combination of criteria from a patient health file that be algorithmically identified and to whom a kit may be automatically sent, or may be flagged to be sent a communication, or placed on a high-risk list for insurers. In some embodiments, the need for a UV skin damage assessment is determined using a mobile communication device such as a cell phone. For example, the subject may take a picture on a cell phone, the image may be analyzed, and a recommendation to have a UV skin damage assessment may be returned to the subject.

Some embodiments include monitoring a subject using a method as described herein. For example, the presence or extent of UV skin damage may be determined multiple times based on the expression levels of one or more target genes at separate time points. Some embodiments include comparing UV skin damage in sequentially obtained samples. In some embodiments, a kit is provided that includes a space kit for “before” and “after” samples differentially labeled, useful for those undergoing specific treatments. In some embodiments, the multiple UV skin assessments are performed about a month or more apart. Some embodiments include performing the assessment again after 1 month, 2 months, 3 months, 4 months, 5 months, 6 months, 7 month, 8 months, 9 months, 10 months, 11 months, 12 months, or more, or a range of months including any two of the aforementioned numbers of months. Some embodiments include performing the assessment again after at least 30 days. Some embodiments include testing sequentially, or may include looking for incremental changes in UV skin damage. Some embodiments include performing a method as provided herein to determine the presence or extent of skin damage before and/or after (e.g. 30 or more days after) a laser treatment, chemical peel or other treatment. In some cases, the UV skin assessment is used to determine a pass/fail, or to show a positive or negative impact of a particular skin treatment. For example, a pass or improvement may include an increase or decrease in one or more target genes, such as a 2×, 5×, or OX improvement in the up/downregulation of the target gene(s).

Disclosed herein, in certain embodiments, is a method of monitoring ultraviolet (UV) skin damage. Some embodiments include isolating nucleic acids from a first skin sample obtained from a subject at a first time. In some embodiments, the nucleic acids are isolated from the first skin sample by applying an adhesive patch to a skin region of the subject in a manner sufficient to adhere skin sample cells to the adhesive patch, and removing the adhesive patch from the first skin sample in a manner sufficient to retain the adhered skin sample cells to the adhesive patch. In some embodiments, the skin sample cells from the first skin sample comprise cells from the stratum corneum. Some embodiments include measuring or detecting an expression level of one or more target genes known to be upregulated or downregulated in subjects with UV skin damage, in the first skin sample. Some embodiments include determining a presence or an amount of UV skin damage in the first skin sample based on the expression level of the one or more target genes. Some embodiments include isolating nucleic acids from a skin sample obtained from the subject at a second time. Some embodiments include measuring or detecting an expression level of the one or more target genes in the second skin sample. In some embodiments, the nucleic acids are isolated from the second skin sample by applying an adhesive patch to a skin region of the subject in a manner sufficient to adhere skin sample cells to the adhesive patch, and removing the adhesive patch from the second skin sample in a manner sufficient to retain the adhered skin sample cells to the adhesive patch. In some embodiments, the skin sample cells from the second skin sample comprise cells from the stratum corneum. Some embodiments include determining a presence or an amount of UV skin damage in the second skin sample based on the expression level of the one or more target genes. Some embodiments include comparing the presence or amount of UV skin damage in the second skin sample to the presence or amount of UV skin damage in the first skin sample. Some embodiments include providing a skin treatment to the subject after the first skin sample is obtained, and before the second skin sample is obtained. In some embodiments, the skin treatment comprises a sunscreen. Some embodiments include an efficacy of the skin treatment based on the comparison of the presence or amount of UV skin damage in the second skin sample to the presence or amount of UV skin damage in the first skin sample. An advantage of using target genes to assess UV skin damage is that it does not, in some instances, require a determination of erythema in the subject. Thus, in some embodiments, the assessment is more quantitative and/or less subjective than erythema assessment. In some embodiments, the efficacy of the skin treatment comprises a SPF. For example, some embodiments relate to using target gene expression levels, or a UV exposure score, to evaluate a sun protection factor (SPF) of a product. Some embodiments include using target gene expression levels to evaluate a sun protection factor (SPF) of a product, to evaluate an SPF-equivalent of the product, or to evaluate a sun protection score of the product. Some embodiments include using target gene expression levels to determine a sun protection factor (SPF) of a product, to determine an SPF-equivalent of the product, or to determine a sun protection score of the product. The product may be a sunscreen or a lip balm, but is not limited to such embodiments. Some embodiments include providing a second skin treatment to the subject. Some embodiments include providing a second skin treatment to the subject after second skin sample is obtained. Some embodiments include providing a second skin treatment to the subject after second skin sample is obtained, based on the presence or amount of UV skin damage in the second skin sample compared to the presence or amount of UV skin damage in the first skin sample. Some embodiments include providing a second skin treatment to the subject after the second skin sample is obtained, when there is UV skin damage or an amount of UV skin damage above a threshold, or greater than a control amount. Some embodiments include not providing a second skin treatment to the subject after the second skin sample is obtained, when there is not UV skin damage or when an amount of UV skin damage below a threshold, or lower than a control amount. Some embodiments include not providing a second skin treatment to the subject after the second skin sample is obtained, when there is UV skin damage or an amount of UV skin damage above a threshold, or greater than a control amount. Some embodiments include providing a second skin treatment to the subject after the second skin sample is obtained, when there is not UV skin damage or when an amount of UV skin damage below a threshold, or lower than a control amount.

### UV Exposure Scores

Disclosed herein, in some embodiments, is a UV exposure score. In some embodiments, the UV exposure score is used in a method described herein. For example, the UV exposure score may be used in a method of detecting, assessing, measuring, or determining the presence of a skin damage such as UV skin damage. In some embodiments, the UV exposure score incorporates the expression level of one or more target genes described herein. Based on a patient's UV exposure score, they may be treated with, or recommended treatment with a skin treatment described herein. In some embodiments, the UV exposure score is generated with a computer or processor. In some embodiments, the UV exposure score is provided to a medical practitioner. In some embodiments, the UV exposure score is provided to a patient or subject.

In some embodiments, the UV exposure score comprises an integer indicative of UV exposure. In some embodiments, the UV exposure, or the UV exposure score, is indicative of sun damage. In some embodiments, the UV exposure score is indicative of UV skin damage. In some cases, a higher UV exposure score indicates more UV exposure or more UV skin damage than a lower score. In some cases, a lower UV exposure score indicates less UV exposure or less UV skin damage than a higher score. Examples of UV exposure scores include integers from 1 to 10. In some embodiments, the UV exposure score is 1, 2, 3, 4, 5, 6, 7, 8, 9, or 10. In some embodiments, the UV exposure score is in a range defined by any two of. 1, 2, 3, 4, 5, 6, 7, 8, 9, or 10.

The UV exposure score may quantitative (e.g., numeric or alphanumeric), with higher or lower resolution (e.g., 1-10 or high/medium/low), or qualitative (e.g., significant increase/decrease relative to a cohort), or the like. In some embodiments, the UV exposure score is quantitative. In some embodiments, the quantitative UV exposure score is numeric. In some embodiments, the quantitative UV exposure score is alphanumeric. In some embodiments, the quantitative UV exposure score is alphabetic. In some embodiments, the quantitative UV exposure score is a value or a range of values such as 1-10 or A-Z. In some embodiments, the quantitative UV exposure score is relative or general, for example: “low,” “medium,” or “high.” In some embodiments, the quantitative UV exposure score is relative to a control UV exposure score, or relative to a baseline (e.g. pre-exposure) UV exposure score.

In some embodiments, the UV exposure score is qualitative. In some embodiments, the qualitative UV exposure score is numeric. In some embodiments, the qualitative UV exposure score is “yes” or “no.” In some embodiments, the qualitative UV exposure score is “significant” or “insignificant.” In some embodiments, the qualitative UV exposure score is a significant increase or decrease relative to a control such as a cohort. In some embodiments, the qualitative UV exposure score is relative to a control UV exposure score, or relative to a baseline (e.g. pre-exposure) UV exposure score.

In some embodiments, the UV exposure score incorporates the expression level of one or more target genes. In some embodiments, the one or more target genes comprises an ADAMTSL Family member, a CDKN Family member, a CST Family member, a KIF Family member, a MKI Family member, a SLAM Family member, a TRIP Family member, a UHRF Family member, a Vitamin A Family member, an Interleukin Family member, a KLK Family member, a KRT Family member, a MUCL Family member, a PDCD Family member, a SPRR Family member, or a combination thereof, as disclosed herein. In some embodiments, the UV exposure score incorporates a gene classifier disclosed herein.

In some embodiments, an algorithm evaluates the various expression levels and make assumptions or recommendations. In some embodiments, the algorithm uses gene expression data, and/or patient parameters such as age, sex, skin type, history of sun damage, tanning bed use, smoking, sunburns.

In some embodiments, the UV exposure score incorporates an assessment of a subject's age, sex, skin type, history of sun damage, tanning bed use, smoking, or visible sunburn status. In some embodiments, the UV exposure score incorporates an assessment of a subject's age, smoking history, place of residence, occupation, or medical history. In some embodiments, the UV exposure score incorporates an assessment of a subject's age, gender, and/or skin condition. In some embodiments, the UV exposure score incorporates an assessment of a subject's smoking history. In some embodiments, the UV exposure score incorporates an assessment of a subject's place of residence. In some embodiments, the UV exposure score incorporates an assessment of a subject's occupation. In some embodiments, the UV exposure score incorporates an assessment of a subject's medical history. In some embodiments, the UV exposure score incorporates an assessment of a subject's skin condition. In some embodiments, the UV exposure score incorporates an assessment of a subject's history of sun damage. In some embodiments, the UV exposure score incorporates an assessment of a subject's tanning bed use. In some embodiments, the UV exposure score incorporates a visual assessment of a subject's skin damage. In some embodiments, the assessment of a subject's skin damage includes an image of the subject's skin. In some embodiments, the UV exposure score incorporates an assessment of a subject's erythema. In some embodiments, the assessment of a subject's erythema includes an erythema grade.

In some embodiments, the UV exposure score incorporates a subject's age. In some embodiments, the UV exposure score is normalized based on the subject's age. In some embodiments, the UV exposure score is increased based on the subject's age. In some embodiments, the UV exposure score is decreased based on the subject's age.

In some embodiments, the UV exposure score incorporates a subject's gender. In some embodiments, the UV exposure score is normalized based on the subject's gender. In some embodiments, the UV exposure score is increased based on the subject's gender. In some embodiments, the UV exposure score is decreased based on the subject's gender.

In some embodiments, the UV exposure score incorporates an assessment of a subject's skin condition. In some embodiments, the skin condition is visually assessed and/or scored. In some embodiments, the UV exposure score is increased based on the subject's skin condition, such as a poor skin condition and/or erythema. In some embodiments, the UV exposure score is decreased based on the subject's skin condition, such as a good skin condition and/or lack of erythema.

In some embodiments, the UV exposure score incorporates an assessment of a subject's skin type. For example, skin type may be used to categorize the level or pigmentation in skin. This level may be used in algorithm to generate the score.

Some embodiments of the methods described herein include analyzing a plurality of target genes (e.g. 2 or more target genes) using skin patch collection methodology for gene expression analysis to obtain gene expression data. Some embodiments include analyzing or algorithmically analyzing the gene expression data by statistically analyzing the gene expression data. Some embodiments include determining a correlation of at least two of the target genes. In some embodiments, the correlation is linear. In some embodiments, the correlation is logistic. In some embodiments, the correlation is exponential. In some embodiments, the correlation is a Pearson correlation. Some embodiments include classifying data using regression. In some embodiments, the regression is logistic. In some embodiments, the regression is linear. In some embodiments, the regression is exponential. Some embodiments include analyzing or algorithmically analyzing the gene expression data by statistically analyzing the gene expression data and/or other variables such as clinical parameters. In some embodiments, some of the gene expressions or other variables are correlated with each other, and their statistical dependence is considered when analyzing the data. In some embodiments, the analysis includes correlating the expression levels of at least two of the plurality of target genes. In some embodiments, the analysis includes classifying data based on a regression. Some embodiments include calculating a UV exposure score based on the analyzed gene expression data. Some embodiments of the methods described herein include analyzing a plurality of target genes using skin patch collection methodology for gene expression analysis to obtain gene expression data; algorithmically analyzing the gene expression data by statistically analyzing the gene expression data; and calculating a UV exposure score based on the analyzed gene expression data. In some embodiments, the gene expression data is from target genes, or from a gene classifier, as described herein. Some embodiments include comparing the subject's UV exposure score to a population UV exposure score range. Some embodiments include outputting the UV exposure score (for example, to a report, health database, healthcare practitioner, or subject). Some embodiments include recommending a skin treatment for the subject (e.g., in the report or health database, or to the healthcare practitioner or patient).

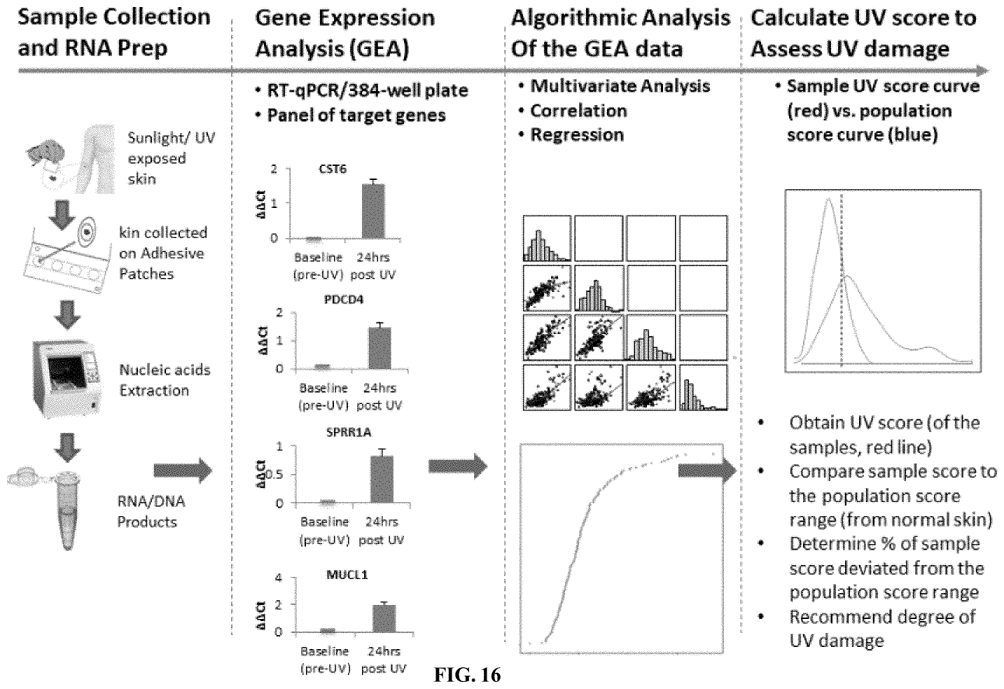

UV exposure scores may be used to determine UV exposure produced synthetically or by the sun. FIG. 16 provides non-limiting exemplary workflow processes that may be used in such a method, or for another method described herein. Some non-limiting examples of uses of the UV exposure score include testing of sunscreen products and UV exposure supplements, and monitoring of sun exposure in patients and consumers.

Provided herein are methods of assessing and monitoring UV exposure in a subject. Some embodiments of the methods described herein include producing a UV exposure score for a patient based on the levels of the one or more target genes. In some embodiments, determining a UV exposure score comprises determining a probability that a subject has UV skin damage based on the levels of the one or more target genes.

In some embodiments, producing a UV exposure score comprises applying a mathematical algorithm to the target gene expression levels. In some embodiments, the production of the UV exposure score is performed by a processor and cannot practically be performed in a human mind. For example, in some embodiments, some calculations performed by the algorithm may not be practically performed by the human mind. In some embodiments, the methods described herein provide a significant advantage in computer processing, assessment of UV skin damage, and patient treatment, over conventional methods. For example, the methods and systems provided herein may provide benefits in patient monitoring over conventional methods of patient monitoring, or aid in speeding up computer processing.

In some embodiments, the UV exposure score incorporates a target gene measurement. In some embodiments, the target gene is compared to a reference or control target gene measurement. In some embodiments, the target gene is compared to a reference target gene measurement. In some embodiments, the target gene is compared to a control target gene measurement. In some embodiments, the target gene is compared to multiple reference or control target gene measurements. In some embodiments, the target gene measurement is entered into a model, such as a regression model, relating the to an amount of UV skin damage. In some embodiments, the target gene measurement is entered into multiple models. The reference or control target gene measurements can include ranges of values. In some embodiments, the reference or control target gene measurement is from a control patient with a known amount of UV skin exposure or UV damage. In some embodiments, the UV exposure score is relative to a control UV exposure score, or relative to a baseline (e.g. pre-exposure) UV exposure score.

Disclosed herein, in some embodiments, is a method of producing a UV exposure score. In some embodiments, the method comprises obtaining expression levels of target genes in a skin sample obtained from a subject. Some embodiments include generating a UV exposure score for the subject. Some embodiments include comparing the expression levels to a model. In some embodiments, the model is derived from target gene expression levels in skin samples from a cohort of subjects. In some embodiments, the model is derived from amounts UV skin damage or exposure in the cohort of subjects. In some embodiments, the model is derived from target gene expression levels in skin samples from a cohort of subjects, and is derived from amounts UV skin damage or exposure in the cohort of subjects. Some embodiments include generating a UV exposure score for the subject by comparing the expression levels to a model derived from target gene expression levels in skin samples from a cohort of subjects, and derived from amounts UV skin damage or exposure in the cohort of subjects. In some embodiments, the model comprises a random forest model. In some embodiments, comprises a boosting model. In some embodiments, the model comprises a lasso model. In some embodiments, the model comprises a logistic model. In some embodiments, the model comprises a random forest model, a boosting model, a lasso model, and/or a logistic model. In some embodiments, the model is derived using regression. In some embodiments, the model is derived using random forest classification. In some embodiments, the model is derived using logistic regression. In some embodiments, the model is derived using quantile classification. In some embodiments, the model is derived using ordinary least squares regression. In some embodiments, the model is derived using classification and regression trees.

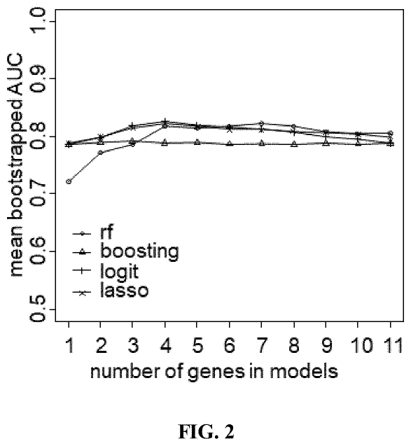

In some embodiments, a multivariate analysis is performed to reduce a number of possible variables. In some embodiments, the analysis weighs multiple variables (which may be single target genes or interactions of target genes) based on a p-value or area under the curve (AUC) value of each individual factor. In some embodiments, the analysis puts the variables together to calculate an overall AUC value. As the overall AUC values may change with the number of variables used for the calculation, in some embodiments this produces one or more AUC curves. The one or more AUC curves may be visualized graphically (e.g. with the AUC value on y-axis, and the number of variables on x-axis and also shown in the gene table, see, e.g., FIG. 2). In some embodiments, a gene table ranks the importance of each variable from top to bottom (e.g. 1 to 16). Various models may be used for calculation of the overall AUC values with the number of variables. In some embodiments, 1-4 models used (random forest (rf), boosting, lasso, logistic). In, for example, FIG. 2 and some other figures, 4 models were used, and so 4 AUC curves may be shown in the AUC figures, and 4 columns of variables in some gene tables (e.g. Table 7). In some embodiments, AUC values on the y-axis include accumulative AUC values, with increased number of variables shown on the x-axis. In some embodiments, a higher AUC may mean a better test (given a better separation of 2 groups examined, e.g., UV skin damage vs. non-UV skin damage). In some embodiments, the best (or the highest) AUC is picked from the AUC curves (e.g. from AUC curves shown on an AUC figure) (regardless the models), and a number of variables (one-axis) is identified that gives this best AUC. In some embodiments, genes from the variables will make up a gene panel for a UV skin exposure test (e.g. a method incorporating target genes). In some embodiments, an overall AUC is calculated, individual target genes and/or interactions of target genes are included.

Relationships between the target gene expression levels and the UV skin exposure may be derived by any of a number of statistical processes or statistical analysis techniques. In some embodiments, logistic regression is used to derive one or more equations of the mathematical algorithm. In some embodiments, linear regression is used to derive one or more equations of the algorithm. In some embodiments, ordinary least squares regression or unconditional logistic regression is used to derive one or more equations of the algorithm. Some embodiments include a computer system that performs a method described herein, or steps of a method described herein. Some embodiments include a computer-readable medium with instructions for performing all or some of the various steps of the methods and systems provided herein. In some embodiments, the logistic regression comprises backward elimination. In some embodiments, the logistic regression comprises Akike information criterion.

Some embodiments include developing or training a model. In some embodiments, the model is an algorithm such as a UV exposure score algorithm. In some embodiments, the model is developed by testing candidate target gene expression levels. In some embodiments, the model is developed by testing candidate target gene expression levels from skin samples known to have UV skin damage. In some embodiments, the model is developed by testing candidate target gene expression levels from skin samples known to have a specific amount of UV skin damage. In some embodiments, an analytical method validation (AMV) is performed on a target gene panel. In some embodiments, multiple logistic regression is used to predict UV skin damage as a function of skin target gene expression levels. Some embodiments include logarithmic transformation and/or combined through backward elimination with Akaike information criterion (AIC). In some embodiments, a UV exposure score model is obtained by transforming a logistic function in terms of probability to have UV skin damage. Some embodiments include transforming a logistic function of each target gene to a probability such as a probability of having UV skin damage. Some embodiments include combining one or two logistic functions or models to product the probability. Some embodiments include generating a UV exposure score based on an input of probabilities generated for each target gene expression level.

In some embodiments, continuous variables are reported as medians with interquartile ranges (IQR), and compared between groups using the Mann-Whitney test. In some embodiments, categorical variables are reported as numbers (n) and percentages (%), and compared between groups using a Fisher's exact test. In some embodiments, a Delong method is used to compute a 95% confidence interval (CI) of AUROC, and/or to compare AUROCs of different target genes on paired samples. In some embodiments, exact binomial confidence limits are used for the 95% CIs of sensitivity and specificity. In some embodiments, the 95% CIs of PLR and NLR are computed. In some embodiments, a pairwise Wilcoxon rank sum test is used for comparing effect size of different variables. In some embodiments, a p value (e.g. one-sided or two-sided) of 0.05 or lower is considered as significant.

In some embodiments, applying the mathematical algorithm to the target gene expression levels comprises using one, two, three, or more models relating the levels of the target genes to a UV exposure score. In some embodiments, results are generated from more than one model. In some embodiments, the results comprise a probability such as a probability of a patient having UV skin damage. In some embodiments, the results generated from each of the more than one model are averaged. In some embodiments, producing an exposure score for the patient comprises using one, two, three, or more models relating the levels of the target genes to a known amount of UV skin damage. In some embodiments, the mathematical algorithm comprises a model relating the levels of the target genes to a known amount of UV skin damage. In some embodiments, the mathematical algorithm comprises two or more models relating the levels of the target genes to a known amount of UV skin damage. In some embodiments, one or more of the models are derived by using classification and regression trees, and/or one or more of the models are derived by using ordinary least squares regression to model diagnostic specificity. In some embodiments, one or more of the models are derived by using random forest learning classification, and/or one or more of the models are derived by using quantile classification. In some embodiments, one or more of the models are derived by using logistic regression to model diagnostic sensitivity, and/or one or more of the models are derived by using logistic regression to model diagnostic specificity. In some embodiments, the use of two or more models provides an unexpected benefit of increasing sensitivity in relating the UV exposure score to the known amount of UV skin damage. In some embodiments, the use of two or more models provides an unexpected benefit of increasing specificity in relating the target gene expression levels to the known amount of UV skin damage.

In some embodiments, the statistical analyses includes a quantile measurement of one or more target genes. Quantiles can be a set of “cut points” that divide a sample of data into groups containing (as far as possible) equal numbers of observations. For example, quartiles can be values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The algorithm can also include the use of percentile ranges of target gene expression levels (e.g., tertiles, quartile, quintiles, etc.), or their cumulative indices (e.g., quartile sums of target gene expression levels to obtain quartile sum scores (QSS), etc.) as variables in the statistical analyses (just as with continuous variables).

In some embodiments, the statistical analyses include one or more learning statistical classifier systems. As used herein, the term “learning statistical classifier system” includes a machine learning algorithmic technique capable of adapting to complex data sets (e.g., panel of target genes of interest) and making decisions based upon such data sets. In some embodiments, a single learning statistical classifier system such as a decision/classification tree (e.g., random forest (RF) or classification and regression tree (C&RT)) is used. In some embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as RF, C&RT, boosted trees, etc.), Probably Approximately Correct (PAC) learning, connectionist learning (e.g., neural networks (NN), artificial neural networks (ANN), neuro fuzzy networks (NFN), network structures, the Cox Proportional-Hazards Model (CPHM), perceptrons such as multi-layer perceptrons, multi-layer feed-forward networks, applications of neural networks, Bayesian learning in belief networks, etc., reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ).

Random forests are learning statistical classifier systems that are constructed using an algorithm developed by Leo Breiman and Adele Cutler. Random forests use a large number of individual decision trees and decide the class by choosing the mode (i.e., most frequently occurring) of the classes as determined by the individual trees.

Classification and regression trees represent a computer intensive alternative to fitting classical regression models and are typically used to determine the best possible model for a categorical or continuous response of interest based upon one or more predictors. In some embodiments, the statistical methods or models are trained or tested using a cohort of samples (e.g., skin samples) from healthy individuals with and without UV skin damage.

In certain aspects, one or more equations of the mathematical algorithm are derived to model diagnostic sensitivity, e.g., the proportion of actual positives that are correctly identified as such. For example, one or more equations can be trained using the data to predict an amount of UV skin damage with the measured target gene expression levels. In certain aspects, one or more equations of the mathematical algorithm are derived to model diagnostic specificity, e.g., the proportion of actual negatives that are correctly identified as such. For example, one or more equations can be trained using the data to predict a UV skin damage with the measured target gene expression levels. In some embodiments, the mathematical algorithm includes two or more equations, one or more of which are derived to model diagnostic sensitivity, and one or more of which are derived to model diagnostic specificity. In certain aspects, the mathematical algorithm applies one or more diagnostic sensitivity equations prior to applying one or more diagnostic specificity equations in a sequence to generate a UV exposure score. In certain aspects, the mathematical algorithm applies one or more diagnostic specificity equations prior to applying one or more diagnostic sensitivity equations in a sequence to generate a UV exposure score. In some embodiments, the algorithm is trained based on skin samples known to have UV skin damage and known expression levels of target genes.

Some embodiments of the methods and systems described herein include generating a probability of the patient having UV skin damage by applying a model to at least one target gene expression level. In some embodiments, the probability is 0%, 1%, 5%, 10%, 15%, 20%, 25%, 30%, 35%, 40%, 45%, 50%, 55%, 60%, 65%, 70%, 75%, 80%, 85%, 90%, 95%, 99%, or 100%. In some embodiments, the probability is 0-10%. In some embodiments, the probability is 10-20%. In some embodiments, the probability is 20-30%. In some embodiments, the probability is 30-40%. In some embodiments, the probability is 40-50%. In some embodiments, the probability is 50-60%. In some embodiments, the probability is 60-70%. In some embodiments, the probability is 70-80%. In some embodiments, the probability is 80-90%. In some embodiments, the probability is 90-100%. Some embodiments include generating a probability for each target gene. In some embodiments, each target gene expression level is multiplied by a separate factor. In some embodiments, the probability for each target gene expression level is multiplied by a separate factor. Some embodiments, include generating a probability based on multiple target genes.

In some embodiments, at least one target gene expression level is weighted. In some embodiments, the weight of a target gene expression level is compared to a threshold. In some embodiments, the weight of a target gene expression level is assigned by a computer algorithm. In some embodiments, the weight of a target gene expression level affects how much a particular target gene contributes to calculating a UV exposure score. In some embodiments, the weight of a first target gene expression level is less than the weight of a second target gene expression level. In such cases, the first target gene expression level can be less informative of the UV exposure score than the second target gene. In some embodiments, the weight of a first target gene expression level is greater than the weight of a second target gene expression level. In such cases, the first target gene can be more informative of UV skin damage or the UV exposure score than the second target gene. In some embodiments, each target gene is given a separate weight in the mathematical algorithm. For example, the level of one target gene may have a greater impact on the UV exposure score than another of target gene.

In some embodiments, the weight is 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 50, or 100, in relation to another of the target genes. In some embodiments, the weight is 0.01-0.1 in relation to another of the target genes. In some embodiments, the weight is 0.1-0.5 in relation to another of the target genes. In some embodiments, the weight is 0.5-1 in relation to another of the target genes. In some embodiments, the weight is 1-1.5 in relation to another of the target genes. In some embodiments, the weight is 1.5-2 in relation to another of the target genes. In some embodiments, the weight is 2-10 in relation to another of the target genes. In some embodiments, the weight is 10-100 in relation to another of the target genes. In some embodiments, the a target gene is weighted such that it contributes 0.01, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 50, or 100% of the UV exposure score.

Some embodiments of the methods and systems described herein include based on the weight for the probability generated from each target gene, generating an overall probability of the subject having UV skin damage, or an amount of UV skin damage. In some embodiments, the overall probability is 0%, 1%, 5%10%, 15%, 20%, 25%, 30%, 35%, 40%, 45%, 50%, 55%, 60%, 65%, 70%, 75%, 80%, 85%, 90%, 95%, 99%, or 100%. In some embodiments, the overall probability is 0-10%. In some embodiments, the overall probability is 10-20%. In some embodiments, the overall probability is 20-30%. In some embodiments, the overall probability is 30-40%. In some embodiments, the overall probability is 40-50%. In some embodiments, the overall probability is 50-60%. In some embodiments, the overall probability is 60-70%. In some embodiments, the overall probability is 70-80%. In some embodiments, the overall probability is 80-90%. In some embodiments, the overall probability is 90-100%.

Some embodiments include the use of an intermediate value for one or more target gene expression levels. In some embodiments, the algorithm converts the level of a target gene into an intermediate value for that target gene. In some embodiments, the algorithm converts the level of multiple target genes, or all of the target genes, into intermediate values. In some embodiments, the algorithm converts the expression level of multiple target genes into a single intermediate value. In some embodiments, the intermediate values are converted by the algorithm into the UV exposure score. In some embodiments, the use of an intermediate value improves the speed of producing the UV exposure score from the expression levels, thereby increasing the processing speed of a computer or device implementing the mathematical algorithm. In some embodiments, the use of an intermediate value improves a computer technology or other device.

In some embodiments, a target gene expression level that is less than a reference or control target gene expression level is indicative of UV skin damage. In some embodiments, a target gene expression level that is greater than a reference or control target gene expression level is indicative of UV skin damage. In some embodiments, a target gene expression level that is less than a reference or control target gene expression level is indicative of a lack of UV skin damage. In some embodiments, a target gene expression level that is greater than a reference or control target gene expression level is indicative of a lack of UV skin damage. In some embodiments, a target gene expression level that is less than a reference or control target gene expression level is indicative of an amount of UV skin damage. In some embodiments, a target gene expression level that is greater than a reference or control target gene expression level is indicative of an amount of UV skin damage.

In some embodiments, a computer or processor applies a mathematical algorithm to the target gene expression levels. In some embodiments, the UV exposure score is produced by or using a computer or processor. In some embodiments, the computer or processor receives the target gene expression levels. In some embodiments, a user enters the target gene expression levels, for example into a graphical user interface. In some embodiments, the computer or processor implements the mathematical algorithm to generate the UV exposure score. In some embodiments, the computer or processor performs or is used to perform one, more, or all steps of the method. In some embodiments, the computer or processor displays the UV exposure score. In some embodiments, the computer or processor transmits the UV exposure score, for example over a network to another computer or processor. Some embodiments include receiving the UV exposure score.

Some embodiments of the methods described herein include obtaining or generating a UV exposure score for a subject. Some embodiments include comparing the UV exposure score for the subject to a reference score such as a population score. The population score may include scores or a score range for subjects with UV skin damage. The population score may include scores or a score range for subjects with various amounts of UV skin damage (e.g. quantile amounts of UV skin damage or gene expression levels, and UV expression scores or score ranges delineating each quantile). The population score may include scores or a score range for subjects without UV skin damage. Some embodiments include determining an amount of deviation of the UV exposure score for the subject compared to a population UV exposure score or score range. For example, some embodiments include determining a percent of deviation of the UV exposure score for the subject compared to the population UV exposure score or score range. In some embodiments, the population UV exposure score or score range includes an average UV exposure score, or a quantile UV exposure score such as a quartile or quintile UV exposure score. Some embodiments include indicating a degree of UV damage for the subject based on the UV exposure score for the subject. Such indications may come in the form of a recommendation, a determination, or a communication about the determination or recommendation.

Some embodiments relate to a method that includes using a UV exposure score to evaluate a sun protection factor (SPF) of a product, to evaluate an SPF-equivalent of the product, or to evaluate a sun protection score of the product. Some embodiments include using a UV exposure score to determine a SPF of a product, to determine an SPF-equivalent of the product, or to determine a sun protection score of the product. The product may be a sunscreen or a lip balm, but is not limited to such embodiments.

In some embodiments, the UV exposure score is informative of UV skin damage. In some embodiments, the UV exposure score is informative of an amount of UV skin damage. In some embodiments, the UV exposure score is informative of UV skin exposure. In some embodiments, the UV exposure score is informative of an amount of UV skin exposure. The UV exposure scores may be used in the methods described herein.

Some embodiments relate to a method comprising one or more of the following steps: Step 1) analyze a plurality of target genes for gene expression analysis of skin samples collected using skin patch methodology to obtain gene expression data; Step 2) algorithmically analyze gene expression data collected in Step 1 using the method in Steps 2A and 2B; Step 2A) statistically analyze a plurality of collected gene expression data (e.g. from gene families provided herein); Step 2C) combine the expression values of the collected genes by classification or regression algorithms to calculate a UV exposure score; Step 4) (optional) compare patient UV score to population UV score range; Step 5) output the UV score (e.g., to a report, to a database such as a health database, or to a patient; and Step 6) (optional) recommend a treatment. The plurality of target genes may include one or more target genes or target genes from gene families described herein.

### Components of the Skin Collection Kit

In some embodiments, the adhesive patch from the sample collection kit described herein comprises a first collection area comprising an adhesive matrix and a second area extending from the periphery of the first collection area. The adhesive matrix is located on a skin facing surface of the first collection area. The second area functions as a tab, suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some instances, the adhesive patch is handled with gloves to reduce contamination of the adhesive matrix prior to use.

In some embodiments, the first collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some instances, the adhesive patch does not comprise latex, silicone, or both. In some instances, the adhesive patch is manufactured by applying an adhesive material as a liquid-solvent mixture to the first collection area and subsequently removing the solvent. In some embodiments, the adhesive matrix is configured to adhere cells from the stratum corneum of a skin sample.

The matrix material is sufficiently sticky to adhere to a skin sample. The matrix material is not so sticky that is causes scarring or bleeding or is difficult to remove. In some embodiments, the matrix material is comprised of a transparent material. In some instances, the matrix material is biocompatible. In some instances, the matrix material does not leave residue on the surface of the skin after removal. In certain instances, the matrix material is not a skin irritant.

In some embodiments, the adhesive patch comprises a flexible material, enabling the patch to conform to the shape of the skin surface upon application. In some instances, at least the first collection area is flexible. In some instances, the tab is plastic. In an illustrative example, the adhesive patch does not contain latex, silicone, or both. In some embodiments, the adhesive patch is made of a transparent material, so that the skin sampling area of the subject is visible after application of the adhesive patch to the skin surface. The transparency ensures that the adhesive patch is applied on the desired area of skin comprising the skin area to be sampled. In some embodiments, the adhesive patch is between about 5 and about 100 mm in length. In some embodiments, the first collection area is between about 5 and about 40 mm in length. In some embodiments, the first collection area is between about 10 and about 20 mm in length. In some embodiments the length of the first collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm. In some embodiments, the first collection area is elliptical.

In further embodiments, the adhesive patch of this invention is provided on a peelable release sheet in the adhesive skin sample collection kit. In some embodiments, the adhesive patch provided on the peelable release sheet is configured to be stable at temperatures between −80° C. and 30° C. for at least 6 months, at least 1 year, at least 2 years, at least 3 years, and at least 4 years. In some instances, the peelable release sheet is a panel of a tri-fold skin sample collector.

In some instances, nucleic acids are stable on adhesive patch or patches when stored for a period of time or at a particular temperature. In some instances, the period of time is at least or about 1 day, 2 days, 3 days, 4 days, 5 days, 6 days, 7 days, 2 weeks, 3 weeks, 4 weeks, or more than 4 weeks. In some instances, the period of time is about 7 days. In some instances, the period of time is about 10 days. In some instances, the temperature is at least or about −80° C., −70° C., −60° C., −50° C., −40° C., −20° C., −10° C., −4° C., 0° C., 5° C., 15° C., 18° C., 20° C., 25° C., 30° C., 35° C., 40° C., 45° C., 50° C., or more than 50° C. The nucleic acids on the adhesive patch or patches, in some embodiments, are stored for any period of time described herein and any particular temperature described herein. For example, the nucleic acids on the adhesive patch or patches are stored for at least or about 7 days at about 25° C., 7 days at about 30° C., 7 days at about 40° C., 7 days at about 50° C., 7 days at about 60° C., or 7 days at about 70° C. In some instances, the nucleic acids on the adhesive patch or patches are stored for at least or about 10 days at about −80° C.

The peelable release sheet, in certain embodiments, is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the peelable release sheet is configured to hold about 12 adhesive patches. In some instances, the peelable release sheet is configured to hold about 11 adhesive patches. In some instances, the peelable release sheet is configured to hold about 10 adhesive patches. In some instances, the peelable release sheet is configured to hold about 9 adhesive patches. In some instances, the peelable release sheet is configured to hold about 8 adhesive patches. In some instances, the peelable release sheet is configured to hold about 7 adhesive patches. In some instances, the peelable release sheet is configured to hold about 6 adhesive patches. In some instances, the peelable release sheet is configured to hold about 5 adhesive patches. In some instances, the peelable release sheet is configured to hold about 4 adhesive patches. In some instances, the peelable release sheet is configured to hold about 3 adhesive patches. In some instances, the peelable release sheet is configured to hold about 2 adhesive patches. In some instances, the peelable release sheet is configured to hold about 1 adhesive patch.

Provided herein, in certain embodiments, are methods and compositions for obtaining a sample using an adhesive patch, wherein the adhesive patch is applied to the skin and removed from the skin. After removing the used adhesive patch from the skin surface, the patch stripping method, in some instances, further comprise storing the used patch on a placement area sheet, where the patch remains until the skin sample is isolated or otherwise utilized. In some instances, the used patch is configured to be stored on the placement area sheet for at least 1 week at temperatures between −80° C. and 30° C. In some embodiments, the used patch is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some instances, the placement area sheet comprises a removable liner, provided that prior to storing the used patch on the placement area sheet, the removable liner is removed. In some instances, the placement area sheet is configured to hold a plurality of adhesive patches, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. In some instances, the placement area sheet is configured to hold about 12 adhesive patches. In some instances, the placement area sheet is configured to hold about 11 adhesive patches. In some instances, the placement area sheet is configured to hold about 10 adhesive patches. In some instances, the placement area sheet is configured to hold about 9 adhesive patches. In some instances, the placement area sheet is configured to hold about 8 adhesive patches. In some instances, the placement area sheet is configured to hold about 7 adhesive patches. In some instances, the placement area sheet is configured to hold about 6 adhesive patches. In some instances, the placement area sheet is configured to hold about 5 adhesive patches. In some instances, the placement area sheet is configured to hold about 4 adhesive patches. In some instances, the placement area sheet is configured to hold about 3 adhesive patches. In some instances, the placement area sheet is configured to hold about 2 adhesive patches. In some instances, the placement area sheet is configured to hold about 1 adhesive patch.

The used patch, in some instances, is stored so that the matrix containing, skin facing surface of the used patch is in contact with the placement area sheet. In some instances, the placement area sheet is a panel of the tri-fold skin sample collector. In some instances, the tri-fold skin sample collector further comprises a panel. In some instances, the tri-fold skin sample collector further comprises a clear panel. In some instances, the tri-fold skin sample collector is labeled with a unique barcode that is assigned to a subject. In some instances, the tri-fold skin sample collector comprises an area for labeling subject information.

In an illustrative embodiment, the adhesive skin sample collection kit comprises the tri-fold skin sample collector comprising adhesive patches stored on a peelable release panel. In some instances, the tri-fold skin sample collector further comprises a placement area panel with a removable liner. In some instances, the patch stripping method involves removing an adhesive patch from the tri-fold skin sample collector peelable release panel, applying the adhesive patch to a skin sample, removing the used adhesive patch containing a skin sample and placing the used patch on the placement area sheet. In some instances, the placement area panel is a single placement area panel sheet. In some instances, the identity of the skin sample collected is indexed to the tri-fold skin sample collector or placement area panel sheet by using a barcode or printing patient information on the collector or panel sheet. In some instances, the indexed tri-fold skin sample collector or placement sheet is sent to a diagnostic lab for processing. In some instances, the used patch is configured to be stored on the placement panel for at least 1 week at temperatures between −80° C. and 25° C. In some embodiments, the used patch is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed tri-fold skin sample collector or placement sheet is sent to a diagnostic lab using UP S or FedEx.

In an exemplary embodiment, the patch stripping method further comprises preparing the skin sample prior to application of the adhesive patch. Preparation of the skin sample includes, but is not limited to, removing hairs on the skin surface, cleansing the skin surface and/or drying the skin surface. In some instances, the skin surface is cleansed with an antiseptic including, but not limited to, alcohols, quaternary ammonium compounds, peroxides, chlorhexidine, halogenated phenol derivatives and quinolone derivatives. In some instances, the alcohol is about 0 to about 20%, about 20 to about 40%, about 40 to about 60%, about 60 to about 80%, or about 80 to about 100% isopropyl alcohol. In some instances, the antiseptic is 70% isopropyl alcohol.

In some embodiments, the patch stripping method is used to collect a skin sample from the surfaces including, but not limited to, the face, head, neck, arm, chest, abdomen, back, leg, hand or foot. In some instances, the skin surface is not located on a mucous membrane. In some instances, the skin surface is not ulcerated or bleeding. In certain instances, the skin surface has not been previously biopsied. In certain instances, the skin surface is not located on the soles of the feet or palms.

The patch stripping method, devices, and systems described herein are useful for the collection of a skin sample from a skin lesion. A skin lesion is a part of the skin that has an appearance or growth different from the surrounding skin. In some instances, the skin lesion is pigmented. A pigmented lesion includes, but is not limited to, a mole, dark colored skin spot and a melanin containing skin area. In some embodiments, the skin lesion is from about 5 mm to about 16 mm in diameter. In some instances, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some instances, the diameter is the longest diameter of the skin lesion. In some instances, the diameter is the smallest diameter of the skin lesion.

The adhesive skin sample collection kit, in some embodiments, comprises at least one adhesive patch, a sample collector, and an instruction for use sheet. In an exemplary embodiment, the sample collector is a tri-fold skin sample collector comprising a peelable release panel comprising at least one adhesive patch, a placement area panel comprising a removable liner, and a clear panel. The tri-fold skin sample collector, in some instances, further comprises a barcode and/or an area for transcribing patient information. In some instances, the adhesive skin sample collection kit is configured to include a plurality of adhesive patches, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet provide the kit operator all of the necessary information for carrying out the patch stripping method. The instructions for use sheet preferably include diagrams to illustrate the patch stripping method.

In some instances, the adhesive skin sample collection kit provides all the necessary components for performing the patch stripping method. In some embodiments, the adhesive skin sample collection kit includes a lab requisition form for providing patient information. In some instances, the kit further comprises accessory components. Accessory components include, but are not limited to, a marker, a resealable plastic bag, gloves and a cleansing reagent. The cleansing reagent includes, but is not limited to, an antiseptic such as isopropyl alcohol. In some instances, the components of the skin sample collection kit are provided in a cardboard box.

In some embodiments, the kit includes a skin collection device. In some embodiments, the skin collection device includes a non-invasive skin collection device. In some embodiments, the skin collection device includes an adhesive patch as described herein. In some embodiments, the skin collection device includes a brush. In some embodiments, the skin collection device includes a swab. In some embodiments, the skin collection device includes a probe. In some embodiments, the skin collection device includes a medical applicator. In some embodiments, the skin collection device includes a scraper. In some embodiments, the skin collection device includes an invasive skin collection device such as a needle or scalpel. In some embodiments, the skin collection device includes a needle. In some embodiments, the skin collection device includes a microneedle. In some embodiments, the skin collection device includes a hook.

Disclosed herein, in some embodiments, are kits for determining the presence of UV skin damage in a skin sample. In some embodiments, the kit includes an adhesive patch. In some embodiments, the adhesive patch comprises an adhesive matrix configured to adhere skin sample cells from the stratum corneum of a subject. Some embodiments include a nucleic acid isolation reagent. Some embodiments include a plurality of probes that recognize at least one target gene. In some embodiments, the at least one target gene is upregulated or downregulated in subjects with UV skin damage. Disclosed herein, in some embodiments, are kits for determining the presence of UV skin damage in a skin sample, comprising: an adhesive patch comprising an adhesive matrix configured to adhere skin sample cells from the stratum corneum of a subject; a nucleic acid isolation reagent; and a plurality of probes that recognize at least one target gene known to be upregulated or downregulated in subjects with UV skin damage.

In some embodiments, the kit is labeled for where the skin sample comes from on the subject (e.g., high UV exposure areas vs low UV exposure areas; or specific sampling locations such as temple, forehead, cheek, or nose). In some embodiments, the adhesive patch is at least 1 cm², at least 2 cm², at least 3 cm², or at least 4 cm², based on the skin sampling location.

Examples of subjects include but are not limited to vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female. In some embodiments, the subject has UV skin damage.

### Cellular Material and Sample Process

In some embodiments of the methods described herein, a skin sample is obtained from the subject by applying an adhesive patch to a skin region of the subject. In some embodiments, the skin sample is obtained using an adhesive patch. In some embodiments, the adhesive patch comprises tape. In some embodiments, the skin sample is not obtained with an adhesive patch. In some instances, the skin sample is obtained using a brush. In some instances, the skin sample is obtained using a swab, for example a cotton swab. In some cases, the skin sample is obtained using a probe. In some cases, the skin sample is obtained using a hook. In some instances, the skin sample is obtained using a medical applicator. In some instances, the skin sample is obtained by scraping a skin surface of the subject. In some cases, the skin sample is obtained through excision. In some instances, the skin sample is biopsied. In some embodiments, the skin sample is a biopsy. In some instances, the skin sample is obtained using one or more needles. For example, the needles may be microneedles. In some instances, the biopsy is a needle biopsy, or a microneedle biopsy. In some instances, the skin sample is obtained invasively. In some instances, the skin sample is obtained non-invasively.

In some embodiments, the skin sample comprises cells of the stratum corneum. In some embodiments, the skin sample consists of cells of the stratum corneum. In some embodiments, the skin sample does not include the basal layer of the skin. In some embodiments, the skin sample comprises or consists of a skin depth of 10 μm, 50 μm, 100 μm, 150 μm, 200 μm, 250 μm, 300 μm, 350 μm, 400 μm, 450 μm, 500 μm, or a range of skin depths defined by any two of the aforementioned skin depths. In some embodiments, the skin sample comprises or consists of a skin depth of 50-100 μm. In some embodiments, the skin sample comprises or consists of a skin depth of 100-200 μm. In some embodiments, the skin sample comprises or consists of a skin depth of 200-300 μm. In some embodiments, the skin sample comprises or consists of a skin depth of 300-400 μm. In some embodiments, the skin sample comprises or consists of a skin depth of 400-500 μm.

In some embodiments, the skin sample is no more than 10 μm thick. In some embodiments, the skin sample is no more than 50 μm thick. In some embodiments, the skin sample is no more than 100 μm thick. In some embodiments, the skin sample is no more than 150 μm thick. In some embodiments, the skin sample is no more than 200 μm thick. In some embodiments, the skin sample is no more than 250 μm thick. In some embodiments, the skin sample is no more than 300 μm thick. In some embodiments, the skin sample is no more than 350 μm thick. In some embodiments, the skin sample is no more than 400 μm thick. In some embodiments, the skin sample is no more than 450 μm thick. In some embodiments, the skin sample is no more than 500 μm thick.

In some embodiments, the skin sample is at least 10 μm thick. In some embodiments, the skin sample is at least 50 μm thick. In some embodiments, the skin sample is at least 100 μm thick. In some embodiments, the skin sample is at least 150 μm thick. In some embodiments, the skin sample is at least 200 μm thick. In some embodiments, the skin sample is at least 250 μm thick. In some embodiments, the skin sample is at least 300 μm thick. In some embodiments, the skin sample is at least 350 μm thick. In some embodiments, the skin sample is at least 400 μm thick. In some embodiments, the skin sample is at least 450 μm thick. In some embodiments, the skin sample is at least 500 M thick.

In some embodiments, the adhesive patch removes a skin sample from the subject at a depth no greater than 10 m. In some embodiments, the adhesive patch removes a skin sample from the subject at a depth no greater than 50 μm. In some embodiments, the adhesive patch removes a skin sample from the subject at a depth no greater than 100 μm. In some embodiments, the adhesive patch removes a skin sample from the subject at a depth no greater than 150 m. In some embodiments, the adhesive patch removes a skin sample from the subject at a depth no greater than 200 μm. In some embodiments, the adhesive patch removes a skin sample from the subject at a depth no greater than 250 μm. In some embodiments, the adhesive patch removes a skin sample from the subject at a depth no greater than 300 m. In some embodiments, the adhesive patch removes a skin sample from the subject at a depth no greater than 350 m. In some embodiments, the adhesive patch removes a skin sample from the subject at a depth no greater than 400 μm. In some embodiments, the adhesive patch removes a skin sample from the subject at a depth no greater than 450 μm. In some embodiments, the adhesive patch removes a skin sample from the subject at a depth no greater than 500 μm.

In some embodiments, the adhesive patch removes 1, 2, 3, 4, or 5 layers of stratum corneum from a skin surface of the subject. In some embodiments, the adhesive patch removes a range of layers of stratum corneum from a skin surface of the subject, for example a range defined by any two of the following integers: 1, 2, 3, 4, or 5. In some embodiments, the adhesive patch removes 1-5 layers of stratum corneum from a skin surface of the subject. In some embodiments, the adhesive patch removes 2-3 layers of stratum corneum from a skin surface of the subject. In some embodiments, the adhesive patch removes 2-4 layers of stratum corneum from a skin surface of the subject. In some embodiments, the adhesive patch removes no more than the basal layer of a skin surface from the subject.

Some embodiments include collecting cells from the stratum corneum of a subject, for instance, by using an adhesive tape with an adhesive matrix to adhere the cells from the stratum corneum to the adhesive matrix. In some embodiments, the cells from the stratum corneum comprise T cells or components of T cells. In some embodiments, the cells from the stratum corneum comprise keratinocytes. In some embodiments, the skin sample does not comprise melanocytes. In some embodiments, a skin sample is obtained by applying a plurality of adhesive patches to a skin region of a subject in a manner sufficient to adhere skin sample cells to each of the adhesive patches, and removing each of the plurality of adhesive patches from the skin region in a manner sufficient to retain the adhered skin sample cells to each of the adhesive patches. In some embodiments, the skin region comprises a skin lesion.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. In some cases, the skin sample adhered to the adhesive matrix comprises or consists of cells from the stratum corneum of a subject. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. In some instances, the nucleic acid is RNA (e.g. mRNA). An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA includes, but not limited to, picogram, nanogram, and microgram quantities. In some embodiments, the RNA includes mRNA. In some embodiments, the RNA includes microRNAs. In some embodiments, the RNA includes mRNA and microRNAs.

The methods and devices provided herein, in certain embodiments, involve applying an adhesive or other similar patch to the skin in a manner so that an effective or sufficient amount of a tissue, such as a skin sample, adheres to the adhesive matrix of the adhesive patch. For example, the effective or sufficient amount of a skin sample is an amount that removably adheres to a material, such as the matrix or adhesive patch. The adhered skin sample, in certain embodiments, comprises cellular material including nucleic acids. In some instances, the nucleic acid is RNA or DNA. An effective amount of a skin sample contains an amount of cellular material sufficient for performing a diagnostic assay. In some instances, the diagnostic assay is performed using the cellular material isolated from the adhered skin sample on the used adhesive patch. In some instances, the diagnostic assay is performed on the cellular material adhered to the used adhesive patch. In some embodiments, an effect amount of a skin sample comprises an amount of RNA sufficient to perform a gene expression analysis. Sufficient amounts of RNA includes, but not limited to, picogram, nanogram, and microgram quantities.

In some instances, the nucleic acid is a RNA molecule or a fragmented RNA molecule (RNA fragments). In some instances, the RNA is a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (IncRNA), a small nuclear RNA (snRNA), a circulating RNA, a cell-free RNA, an exosomal RNA, a vector-expressed RNA, a RNA transcript, a synthetic RNA, or combinations thereof. In some instances, the RNA is mRNA. In some instances, the RNA is cell-free circulating RNA.

In some instances, the nucleic acid is DNA. DNA includes, but not limited to, genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. In some instances, the DNA is genomic DNA. In some instances, the DNA is cell-free circulating DNA.

In additional embodiments, the adhered skin sample comprises cellular material including nucleic acids such as RNA or DNA, in an amount that is at least about 1 picogram. In some embodiments, the amount of cellular material is no more than about 1 nanogram. In further or additional embodiments, the amount of cellular material is no more than about 1 microgram. In still further or additional embodiments, the amount of cellular material is no more than about 1 gram.

In further or additional embodiments, the amount of cellular material is from about 1 picogram to about 1 gram. In further or additional embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In further or additional embodiments, the amount of cellular material, including nucleic acids such as RNA or DNA, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In further or additional embodiments, the amount of cellular material, including nucleic acids such as RNA or DNA, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram.

In some embodiments, isolated RNA from a collected skin sample is reverse transcribed into cDNA, for example for amplification by PCR to enrich for target genes. The expression levels of these target genes are quantified by quantitative PCR in a gene expression test. In some instances, in combination with quantitative PCR, a software program performed on a computer is utilized to quantify RNA isolated from the collected skin sample. In some instances, a software program or module is utilized to relate a quantity of RNA from a skin sample to a gene expression signature, wherein the gene expression signature is associated with a disease such as skin cancer. In some embodiments, a software program or module scores a sample based on gene expression levels. In some embodiments, the sample score is compared with a reference sample score to determine if there is a statistical significance between the gene expression signature and a disease.

In some instances, the layers of skin include epidermis, dermis, or hypodermis. The outer layer of epidermis is the stratum corneum layer, followed by stratum lucidum, stratum granulosum, stratum spinosum, and stratum basale. In some instances, the skin sample is obtained from the epidermis layer. In some cases, the skin sample is obtained from the stratum corneum layer. In some instances, the skin sample is obtained from the dermis.

In some instances, cells from the stratum corneum layer are obtained, which comprises keratinocytes. In some instances, cells from the stratum corneum layer comprise T cells or components of T cells. In some cases, melanocytes are not obtained from the skin sample.

Following extraction of nucleic acids from a biological sample, the nucleic acids, in some instances, are further purified. In some instances, the nucleic acids are RNA. In some instances, the nucleic acids are DNA. In some instances, the RNA is human RNA. In some instances, the DNA is human DNA. In some instances, the RNA is microbial RNA. In some instances, the DNA is microbial DNA. In some instances, human nucleic acids and microbial nucleic acids are purified from the same biological sample. In some instances, nucleic acids are purified using a column or resin based nucleic acid purification scheme. In some instances, this technique utilizes a support comprising a surface area for binding the nucleic acids. In some instances, the support is made of glass, silica, latex or a polymeric material. In some instances, the support comprises spherical beads.

Methods for isolating nucleic acids, in certain embodiments, comprise using spherical beads. In some instances, the beads comprise material for isolation of nucleic acids. Exemplary material for isolation of nucleic acids using beads include, but not limited to, glass, silica, latex, and a polymeric material. In some instances, the beads are magnetic. In some instances, the beads are silica coated. In some instances, the beads are silica-coated magnetic beads. In some instances, a diameter of the spherical bead is at least or about 0.5 um, 1 um, 1.5 um, 2 um, 2.5 um, 3 um, 3.5 um, 4 um, 4.5 um, 5 um, 5.5 um, 6 um, 6.5 um, 7 um, 7.5 um, 8 um, 8.5 um, 9 um, 9.5 um, 10 um, or more than 10 um.

In some cases, a yield of the nucleic acids products obtained using methods described herein is about 500 picograms or higher, about 600 picograms or higher, about 1000 picograms or higher, about 2000 picograms or higher, about 3000 picograms or higher, about 4000 picograms or higher, about 5000 picograms or higher, about 6000 picograms or higher, about 7000 picograms or higher, about 8000 picograms or higher, about 9000 picograms or higher, about 10000 picograms or higher, about 20000 picograms or higher, about 30000 picograms or higher, about 40000 picograms or higher, about 50000 picograms or higher, about 60000 picograms or higher, about 70000 picograms or higher, about 80000 picograms or higher, about 90000 picograms or higher, or about 100000 picograms or higher.

In some cases, a yield of the nucleic acids products obtained using methods described herein is about 100 picograms, 500 picograms, 600 picograms, 700 picograms, 800 picograms, 900 picograms, 1 nanogram, 5 nanograms, 10 nanograms, 15 nanograms, 20 nanograms, 21 nanograms, 22 nanograms, 23 nanograms, 24 nanograms, 25 nanograms, 26 nanograms, 27 nanograms, 28 nanograms, 29 nanograms, 30 nanograms, 35 nanograms, 40 nanograms, 50 nanograms, 60 nanograms, 70 nanograms, 80 nanograms, 90 nanograms, 100 nanograms, 500 nanograms, or higher.

In some cases, methods described herein provide less than less than 10%, less than 8%, less than 5%, less than 2%, less than 1%, or less than 0.5% product yield variations between samples.

In some embodiments, a number of cells is obtained for use in a method described herein. Some embodiments include use of an adhesive patch comprising an adhesive comprising a tackiness that is based on the number of cells to be obtained. Some embodiments include use of a number of adhesive patches based on the number of cells to be obtained. Some embodiments include use of an adhesive patch sized based on the number of cells to be obtained. The size and/or tackiness may be based on the type of skin to be obtained. For example, normal looking skin generally provides less cells and RNA yield than flaky skin. In some embodiments, a skin sample is used comprising skin from a subject's temple, forehead, cheek, or nose. In some embodiments, only one patch is used. In some embodiments, only one patch is used per skin area (e.g. skin area on a subject's temple, forehead, cheek, or nose).

In some cases, methods described herein provide a substantially homogenous population of a nucleic acid product. In some cases, methods described herein provide less than 30%, less than 25%, less than 20%, less than 15%, less than 10%, less than 8%, less than 5%, less than 2%, less than 1%, or less than 0.5% contaminants.

In some instances, following extraction, nucleic acids are stored. In some instances, the nucleic acids are stored in water, Tris buffer, or Tris-EDTA buffer before subsequent analysis. In some instances, this storage is less than 8° C. In some instances, this storage is less than 4° C. In certain embodiments, this storage is less than 0° C. In some instances, this storage is less than −20° C. In certain embodiments, this storage is less than −70° C. In some instances, the nucleic acids are stored for about 1, 2, 3, 4, 5, 6, or 7 days. In some instances, the nucleic acids are stored for about 1, 2, 3, or 4 weeks. In some instances, the nucleic acids are stored for about 1, 2, 3, 4, 5, 6, 7, 8, 9, 10, 11, or 12 months.

In some instances, nucleic acids isolated using methods described herein are subjected to an amplification reaction following isolation and purification. In some instances, the nucleic acids to be amplified are RNA including, but not limited to, human RNA and human microbial RNA. In some instances, the nucleic acids to be amplified are DNA including, but not limited to, human DNA and human microbial DNA. Non-limiting amplification reactions include, but are not limited to, quantitative PCR (qPCR), self-sustained sequence replication, transcriptional amplification system, Q-Beta Replicase, rolling circle replication, or any other nucleic acid amplification known in the art. In some instances, the amplification reaction is PCR. In some instances, the amplification reaction is quantitative such as qPCR.

Provided herein are methods for detecting an expression level of one or more genes of interest from nucleic acids isolated from a biological sample. In some instances, the expression level is detected following an amplification reaction. In some instances, the nucleic acids are RNA. In some instances, the RNA is human RNA. In some instances, the RNA is microbial RNA. In some instances, the nucleic acids are DNA. In some instances, the DNA is human DNA. In some instances, the DNA is microbial DNA. In some instances, the expression level is determined using PCR. In some instances, the expression level is determined using qPCR. In some instances, the expression level is determined using a microarray. In some instances, the expression level is determined by sequencing.

Some embodiments include measuring a microRNA. In some embodiments, the measurement includes use of a stem-loop primer. Some embodiments include the use of poly-A tailing. Some embodiments include a pre-amplification of microRNAs.

FIG. 16 includes non-limiting exemplary cell material and sample processes that may be used in methods described herein.

### Methods of Treatment and Modulation of Gene Expression

Disclosed herein, in some embodiments, are methods of treating a subject suspected of having UV skin damage. Some embodiments include methods of treating a subject with UV skin damage. In some embodiments, the method includes identifying a subject suspected of having the UV skin damage. Some embodiments include determining a treatment regimen for UV skin damage of the subject based on the determined presence or amount of UV skin damage. In some embodiments, the treatment comprises providing a cosmetic regimen. Some embodiments include monitoring treatment efficacy.

Some embodiments relate to making a recommendation or treating a patient in response to the results of a method described herein such as a UV skin damage test. For example, some embodiments include providing or recommending a skin treatment. Some embodiments include not providing or not recommending the skin treatment. In some embodiments, the recommendation or treatment relates to a specific sunscreen or moisturizer for prevention of further damage to, for example, topical agents, chemical peels, lasers, over-the-counter products, or prescription products, for specific treatment depending on the level of damage. In some embodiments, the skin treatment is provided or recommended based on the gene expression levels of one or more target genes, or based on the results of a UV skin damage assessment (e.g. based on a method described herein, or based on a UV exposure score).

Some embodiments include isolating nucleic acids from a skin sample of the subject. In some embodiments, the skin sample is obtained from the subject by applying an adhesive patch to a skin region of the subject. In some embodiments, the adhesive patch is applied in a manner sufficient to adhere skin sample cells. In some embodiments, the skin sample is obtained from the subject further by removing the adhesive patch from the skin sample. In some embodiments, the adhesive patch is removed in a manner sufficient to retain the adhered skin sample cells to the adhesive patch. In some embodiments, the skin sample cells comprise cells from the stratum corneum. In some embodiments, the skin sample cells consist of cells from the stratum corneum. Some embodiments include measuring or detecting an expression level of at least one target gene. The target gene may include any of the target genes s described herein. In some embodiments, the at least one target gene is known to be upregulated or downregulated in subjects with UV skin damage. In some embodiments, the at least one target gene is upregulated or downregulated in the subject. Some embodiments include contacting the isolated nucleic acids with a set of probes that recognize the target gene. Some embodiments include detecting binding between the at least one target gene and the set of probes. In some embodiments, the expression level is detected or measured by contacting the isolated nucleic acids with a set of probes that recognize the target gene, and detecting binding between the at least one target gene and the set of probes. Some embodiments include receiving the expression level of the at least one target gene, wherein the expression level was measured or detected using a method as described herein. Some embodiments include determining whether the subject has UV skin damage based on the expression level of the at least one target gene. Some embodiments include administering a skin damage treatment such as a UV skin damage treatment to the subject. Some embodiments include administering the skin damage treatment to the subject when the subject is determined to have UV skin damage based on the expression level of the at least one target gene. Some embodiments include not administering the skin damage treatment to the subject if the subject is not determined to have UV skin damage based on the expression level of the at least one target gene. Some embodiments include withholding the skin damage treatment from the subject when the subject is not determined to have UV skin damage based on the expression level of the at least one target gene. In some embodiments, the subject has UV skin damage. In some embodiments, the UV skin damage is caused by UVB radiation.

Disclosed herein, in some embodiments, are methods of treating a subject with UV skin damage, comprising: identifying a subject suspected of having UV skin damage; isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; detecting an expression level of at least one target gene known to be upregulated or downregulated in subjects with UV skin damage, by contacting the isolated nucleic acids with a set of probes that recognize the target gene, and detecting binding between the at least one target gene and the set of probes; determining whether the subject has UV skin damage based on the expression level of the at least one target gene; and administering a skin damage treatment to the subject when the subject is determined to have UV skin damage based on the expression level of the at least one target gene, and not administering the skin damage treatment to the subject when the subject is not determined to have UV skin damage based on the expression level of the at least one target gene.

Disclosed herein, in some embodiments, are methods of treating a subject with UV skin damage. Some embodiments include identifying a subject suspected of having UV skin damage. Some embodiments include obtaining a skin sample the subject by applying the adhesive patch to the subject's skin in a manner sufficient to adhere the skin sample to the adhesive patch, and removing the adhesive patch from the subject's skin in a manner sufficient to retain the skin sample adhered to the adhesive patch. Some embodiments include isolating nucleic acids from the skin sample. Some embodiments include contacting the isolated nucleic acids with a set of probes that recognize one or more genes of interest implicated in UV skin damage. Some embodiments include detecting or measuring the amount of binding between the genes of interest and the set of probes. Some embodiments include identifying the subject as having UV skin damage, or as not having UV skin damage, based on the amount of binding between the genes of interest and the set of probes. Some embodiments include administering a treatment for the UV skin damage based on the determination of whether the subject has UV skin damage.

Disclosed herein, in some embodiments, are methods of treating a subject with UV skin damage, comprising: identifying a subject suspected of having UV skin damage; obtaining a skin sample the subject by applying the adhesive patch to the subject's skin in a manner sufficient to adhere the skin sample to the adhesive patch, and removing the adhesive patch from the subject's skin in a manner sufficient to retain the skin sample adhered to the adhesive patch; isolating nucleic acids from the skin sample; contacting the isolated nucleic acids with a set of probes that recognize one or more genes of interest implicated in UV skin damage; detecting or measuring the amount of binding between the genes of interest and the set of probes; identifying the subject as having UV skin damage, or as not having UV skin damage, based on the amount of binding between the genes of interest and the set of probes; and administering a treatment for the UV skin damage based on the determination of whether the subject has UV skin damage.

Disclosed herein, in some embodiments, are methods of treating a subject suspected of having UV skin damage. In some embodiments, the method includes isolating nucleic acids from a skin sample adhered to an adhesive patch. In some embodiments, the skin sample has been obtained from the subject's stratum corneum. Some embodiments include contacting the isolated nucleic acids with a set of probes that recognize target genes. Some embodiments include detecting or measuring an amount of binding between the nucleic acids and the set of probes. Some embodiments include administering to the subject a treatment for UV skin damage when the amount of binding between the nucleic acids and the set of probes is altered in the skin sample relative to a control or threshold amount of binding. Some embodiments include determining that the subject has UV skin damage when the amount of binding between the nucleic acids and the set of probes in the skin sample is altered relative to the control or threshold amount of binding. In some embodiments, the amount of binding between the nucleic acids and the set of probes in the skin sample is greater than the control or threshold amount of binding. In some embodiments, the amount of binding between the nucleic acids and the set of probes in the skin sample is less than the control or threshold amount of binding.

Disclosed herein, in some embodiments, are methods of treating a subject suspected of having UV skin damage, comprising: isolating nucleic acids from a skin sample adhered to an adhesive patch, the skin sample having been obtained from the subject's stratum corneum; contacting the isolated nucleic acids with a set of probes that recognize target genes; detecting or measuring an amount of binding between the nucleic acids and the set of probes; and administering to the subject a treatment for UV skin damage when the amount of binding between the nucleic acids and the set of probes is altered in the skin sample relative to a control or threshold amount of binding.

Described herein, in some embodiments, are methods of treatment that include administering a skin damage treatment to a subject. In some embodiments, the skin damage treatment comprises or consists of a UV skin damage treatment. Some embodiments include administering a skin damage treatment to the subject based on a determination of whether the subject has UV skin damage. Some embodiments include administering a skin damage treatment to the subject based on an extent of UV skin damage. In some embodiments, the skin damage treatment comprises a pharmaceutical composition. In some embodiments, the skin damage treatment comprises a steroid treatment. In some embodiments, the skin damage treatment comprises a surgery. In some embodiments, the skin damage treatment comprises a transplant. In some embodiments, the skin damage treatment comprises an agent for reducing or increasing expression of one or more target genes described herein. In some embodiments, the skin damage treatment comprises vitamin A. In some embodiments, the skin damage treatment comprises a chemical peel. In some embodiments, the skin damage treatment comprises a laser treatment. In some embodiments, the skin damage treatment comprises a topical agent. In some embodiments, the skin damage treatment comprises an over-the-counter product. In some embodiments, the skin damage treatment comprises a prescription, or comprises a prescription product. In some embodiments, the skin damage treatment comprises a cosmetic.

In some embodiments, the skin damage treatment comprises a cosmetic formulation. Some embodiments include providing a cosmetic formulation containing agents for reducing or increasing expression of one or more target genes described herein. In some embodiments, the cosmetic formulation comprises an emulsion, a cream, a lotion, a solution, an anhydrous base, a paste, a powder, a gel, or an ointment. The emulsion may be an oil-in-water emulsion or a water-in-oil emulsion. Alternatively, the formulation may be a solution, such as an aqueous solution or a hydro-alcoholic solution. In another embodiment, the cosmetic formulation is an anhydrous base, such as a lipstick or a powder. In yet another embodiment, the formulation is comprised within an anti-aging product or a moisturizing product. The cosmetic formulation may further contain one or more of estradiol; progesterone; pregnanalone; coenzyme Q10; methylsolanomethane (MSM); copper peptide (copper extract); plankton extract (phytosome); glycolic acid; kojic acid; ascorbyl palmitate; all trans retinol; azaleic acid; salicylic acid; broparoestrol; estrone; adrostenedione; androstanediols; or sunblocks. In some embodiments, the skin damage treatment comprises a lotion. In some embodiments, the skin damage treatment comprises a sunscreen. In some embodiments, the skin damage treatment comprises a hydrogel. In some embodiments, the cosmetic formulation is administered topically.

Some embodiments include 1, 2, 3, 4, 5, 6, 7, 8, 9, 10, 11, 12, 13, 14, or 15, or more administrations of the skin damage treatment. Some embodiments include a range defined by any two of 1, 2, 3, 4, 5, 6, 7, 8, 9, 10, 11, 12, 13, 14, or 15, administrations of the skin damage treatment. Some embodiments include administration daily, weekly, biweekly, or monthly.

In some embodiments, the skin damage treatment includes a pharmaceutical composition. In some embodiments, the pharmaceutical composition is sterile. In some embodiments, the pharmaceutical composition includes a pharmaceutically acceptable carrier. In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

In some embodiments, the skin damage treatment results in prevention, inhibition, or reversion of the UV skin damage in the subject. Some embodiments relate to use of a skin damage treatment herein in the method of preventing, inhibiting, or reversing the UV skin damage. Some embodiments relate to a method of preventing, inhibiting, or reversing UV skin damage in a subject in need thereof. Some embodiments include administering a pharmaceutical composition to a subject with UV skin damage. In some embodiments, the administration prevents, inhibits, or reverses the UV skin damage in the subject. In some embodiments, the pharmaceutical composition prevents, inhibits, or reverses the UV skin damage in the subject.

Some embodiments include administering a skin damage treatment. In some embodiments, administering comprises giving, applying or bringing the skin damage treatment into contact with the subject. In some embodiments, administration is accomplished by any of a number of routes. In some embodiments, administration is accomplished by a topical, oral, subcutaneous, intramuscular, intraperitoneal, intravenous, intrathecal or intradermal route.

In some embodiments, the UV skin damage treatment comprises a DNA repair enzyme. The methods and devices provided herein, in certain embodiments, involve administering a DNA repair enzyme to a subject in need thereof, such as a subject exposed to UVB radiation or suffering from a sun burn. Some embodiments relate to a method of modulating gene or protein expression in the subject. In some embodiments, the DNA repair enzyme is a T4N5 endonuclease. In some embodiments, the DNA repair enzyme is a photolyase. In some embodiments, the UV radiation comprises UVB radiation.

In some embodiments, the DNA repair enzyme is administered to an area of skin exposed to UV radiation. In some embodiments, the DNA repair enzyme is administered to a sunburn or sunburned area of skin on the subject. In some embodiments, the DNA repair enzyme is administered topically.

In some embodiments, the DNA repair enzyme is administered to the subject 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 175, or 200 times, or a range of times defined by any two of the aforementioned numbers. In some embodiments, the DNA repair enzyme is administered daily (e.g. once daily). In some embodiments, the DNA repair enzyme is administered once, twice, three times, four times, or five times daily. In some embodiments, the DNA repair enzyme is administered every 1, 2, 3, 4, 5, 6, or 7 days. In some embodiments, the DNA repair enzyme is administered to the subject for 1, 2, 3, 4, 5, 6, 7, 8, 9, 10, 11, 12, 13, 14, 15, 16, 17, 18, 19, 20, 21, 22, 23, 24, 25 days or weeks, or a range of days or weeks defined by any two of the aforementioned numbers. In some embodiments, the DNA repair enzyme is administered to the subject for two weeks, or for about two weeks.

In some embodiments, the administration modulates expression of one or more gene families or family members, or gene classifiers as described herein. In some embodiments, the administration modulates a gene or protein expression level of CRABP2, IL1RN, IL36G, MUCL1, PDCD4, SPRR1A, CST6, KLK10, or a combination thereof, in the area of skin of the subject where the DNA repair enzyme is administered. In some embodiments, the administration prevents, decreases, or reverses down-regulation of a gene or protein expression level of CRABP2, MUCL1, PDCD4, SPRR1A, CST6, KLK10, or a combination thereof. In some embodiments, the DNA repair enzyme upregulates a gene or protein expression level of CRABP2, MUCL1, PDCD4, SPRR1A, CST6, KLK10, or a combination thereof. In some embodiments, the administration prevents, decreases, or reverses up-regulation of a gene or protein expression level of IL1RN or IL36G. In some embodiments, the DNA repair enzyme down-regulates a gene or protein expression level of IL1RN or IL36G.

In some embodiments, the administration improves or alleviates the sun burn, or a symptom of the sun burn such as itchiness, dryness, cracking, redness, or soreness. In some embodiments, the administration prevents occurrence of a sun burn or symptom thereof.

In some embodiments, the DNA repair enzyme exerts its effects within two weeks of the first administration. For example, the DNA repair enzyme may modulate mRNA expression of CRABP2, IL1RN, IL36G, MUCL1, PDCD4, SPRR1A, CST6, and/or KLK10 over a two week period of time in which the DNA repair enzyme is administered to the skin of the subject daily. In some embodiments, daily administration over a period of time such as 24 hours, two weeks, or 1-14 days, prevents, decreases, or reverses modulation of mRNA expression of CRABP2, IL1RN, IL36G, MUCL1, PDCD4, SPRR1A, CST6, and/or KLK10 by a sunburn. In some embodiments, the DNA repair enzyme is applied or administered to the subject beginning on a day when the subject receives a sunburn or is exposed to UVB radiation. In some embodiments, the DNA repair enzyme is applied or administered to the subject beginning on a day when the subject receives a sunburn or is exposed to UVB radiation. In some embodiments, the DNA repair enzyme is applied or administered to the subject beginning on a day subsequent to the day the subject receives a sunburn or is exposed to UVB radiation.

### Certain Terminologies

Unless defined otherwise, all technical and scientific terms used herein have the same meaning as is commonly understood by one of skill in the art to which the claimed subject matter belongs. It is to be understood that the detailed description are exemplary and explanatory only and are not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

Cellular retinoic acid binding protein 2 (CRABP2), also known as CRABP-II or RBP6, a member of the retinoic acid (RA, a form of vitamin A) binding protein family and lipocalin/cytosolic fatty-acid binding protein family. The CRABP2 protein is a cytosol-to-nuclear shuttling protein, which facilitates RA binding to its cognate receptor complex and transfer to the nucleus. In some instances, CRABP2 has Gene ID: 1382.

Interleukin 1 receptor antagonist (IL1RN), also known as IL1 inhibitor, IRAP, type II interleeeeeukin-1 receptor antagonist, ankinra, IL-Ira3, or DIRA, encodes a member of the interleukin 1 cytokine family that modulates interleukin 1 related immune and inflammatory responses. In some instances, IL1RN has Gene ID: 3557.

Interleukin-36 gamma (IL36G), also known as interleukin-1 homolog 1, interleukin-1 epsilon, interleukin-1 family member 9, interleukin 1-related protein 2, IL-1HH1, IL1RP2, IL1H1, IL1F9, or IL1E, encodes a member of the interleukin 1 cytokine family, in which its activity is stimulated by interferon-gamma, tumor necrosis factor-alpha, and interleukin 1, beta. In some instances, IL36G has Gene ID: 56300.

Small breast epithelial mucin (MUCL1), also known as mucin like 1 or SBEM, encodes a protein that is primary expressed in skin and breast tissues. In some instances, MUCL1 has Gene ID: 118430.

Programmed cell death 4 (PDCD4), also known as neoplastic transformation inhibitor protein, nuclear antigen H731, protein 197/15a, or H731, is a tumor suppressor and encodes a protein that binds to the eukaryotic translation initiation factor 4A1. In some instances, PDCD4 has Gene ID: 27250.

Small proline-rich protein 1A (SPRR1A), also known as cornifin-A, 19 KDa pancornulin, SPR-IA, or SPRK, encodes a protein of the keratinocytes. In some instances, SPRR1A has Gene ID: 6698.

Cystatin E/M (CST6), also known as cysteine proteinase inhibitor, or cystatin 6, encodes a member of the cystatin superfamily of proteins. In some instances, CST6 has Gene ID: 1474.

Kallikrein related peptidase 10 (KLK10), also known as normal epithelial cell-specific 1, protease serine-like 1, PRSSL1, NES1, breast normal epithelial cell associated serine protease, or Kallikrein 10, encodes a protein that belongs to a subgroup of serine proteases. In some instances, KLK10 has Gene ID: 5655.

Interleukin 22 receptor subunit alpha 1 (IL22RA1), also known as cytokine receptor class-II member 9, cytokine receptor family 2 member 9, IL-22R-alpha-1, zcytoR11, CRF2-9, or IL22R, encodes a member of the class II cytokine receptor family of proteins. In some instances, IL22RA1 has Gene ID: 58985.

Interleukin 36 Beta (IL36B), also known as interleukin 1 family member 8, interleukin-1 homolog 2, interleukin-36 beta, interleukin-1 Eta, or IL-1H2, encodes a member of the interleukin 1 cytokine family of proteins. In some instances, IL36B has Gene ID: 27177.

Keratin 17 (KRT17), also known as cytokerain-17, CK-17, PCHC1, or PC2, encodes the keratin 17 protein which is a type Ikeratin. In some instances, KRT17 has Gene ID: 3872.

A disintegrin and metalloproteinase with thrombospondin motifs-like 4 (ADAMTSL4), also known as thrombospondin repeat-containing protein 1, ADAMTS-like protein 4, TSRC1, or ECTOL2, encodes a protein comprising a seven thrombospondin type 1 repeats. The ADAMTSL4 protein is involved in cellular adhesion, angiogenesis, and patterning of the nervous system. In some instances, ADAMTSL4 has Gene ID: 54507.

Cyclin dependent kinase inhibitor 1A (CDKN1A), also known as CDK-interacting protein 1, CAP20, MDA-6, SDI1, WAF1, melanoma differentiation associated protein 6, wild-type P53-activated fragment 1, or P21CIP1, encodes a protein that inhibits the activity of cyclin-cyclin-dependent kinase 2 or -cyclin-dependent kinase 4 complexes. In some instances, CDKN1A has Gene ID: 1026.

Kinesin family member 18B (KIF18B), also known as kinesin-like protein KIF 18B, encodes a protein that is involved in transport along the microtubules. In some instances, KIF18B has Gene ID: 146909.

Marker of proliferation Ki-67 (MKI67), also known as antigen identified by monoclonal antibody Ki-67, protein phosphatase 1, regulatory subunit 105, or PPP1R105, encodes a nuclear protein that is associated with cellular proliferation. In some instances, MKI67 has Gene ID: 4288.

SLAM family member 7 (SLAMF7), also known as CD2 subset 1, protein 19A, CRACC, CS1, novel LY9 (lymphocyte antigen 9) like protein, CD2-like receptor activating cytotoxic cells, novel Ly9, or CD319, encodes a cell surface receptor protein. In some instances, SLAMF7 has Gene ID: 57823.

Thyroid hormone receptor interactor 13 (TRIP13), also known as human papillomavirus type 16 E1 protein-binding protein, thyroid receptor-interacting protein 13, pachytene checkpoint protein 2 homolog, TR-interacting protein 13, 16E1-BP, TRIP-13, MVA3, or PCH2, encodes a protein that interacts with thyroid hormone receptors. In some instances, TRIP13 has Gene ID: 9319.

Ubiquitin like with PHD and ring finger domains 1 (UHRF1), also known as inverted CCAAT box-binding protein of 90 KDa, E3 ubiquitin-protein ligase UHRF1, nuclear zinc finger protein Np95, transcription factor ICBP90, RING finger protein 106, nuclear protein 95, HuNp95, ICBP90, or RNF106, encodes a member of the subfamily of RING-finger type E3 ubiquitin ligases. In some instances, UHRF1 has Gene ID: 29128.

Some embodiments relate to a gene in Table 2. For example, some embodiments include measuring, determining, using, or receiving an expression level for one or more genes in Table 2. Some embodiments of the methods described herein relate to an expression level. In some embodiments, the expression level is the expression level of a gene, for example the expression level of a gene encoding a protein. In some embodiments, the expression level is an amount of mRNA such as a measured amount of mRNA. In some embodiments, the mRNA may be measured in RNA isolated from a skin sample.

TABLE 2

| | Gene name |
|---|---|
| Gene name | symbol |
| ADAMTS like 4 | ADAMTSL4 |
| cyclin dependent kinase inhibitor 1A | CDKN1A |
| cystatin E/M | CST6 |
| kinesin family member 18B | KIF18B |
| marker of proliferation KI-67 | MKI67 |
| SLAM family member 7 | SLAMF7 |
| thyroid hormone receptor interactor 13 | TRIP13 |
| ubiquitin like with PHD and ring finger domains 1 | UHRF1 |
| Cellular retinoic acid binding protein 2 | CRABP2 |
| Interleukin 1 receptor antagonist | IL1RN |
| Interleukin 22 receptor, alpha 1 | IL22RA1 |
| Interleukin 1 family, member 8 (IL1F8), =IL36B | IL36B |
| Interleukin 1 family, member 9 (IL1F9), =IL36G | IL36G |
| Kallikrein 10 | KLK10 |
| Keratin 17 | KRT17 |
| Small breast epithelial mucin | MUCL1 |
| Programmed cell death 4 | PDCD4 |
| Small proline-rich protein 1A | SPRR1A |

Some embodiments disclosed herein relate to UV skin damage. In some embodiments, the UV skin damage is visible. In some embodiments, the UV skin damage comprises is not visually detectable. In some embodiments, the UV skin damage comprises redness. In some embodiments, the UV skin damage comprises blistering. In some embodiments, the UV skin damage comprises soreness. In some embodiments, the UV skin damage comprises a sunburn. In some embodiments, the UV skin damage comprises flakiness. In some embodiments, the UV skin damage comprises peeling. In some embodiments, the UV skin damage comprises dryness. In some embodiments, the UV skin damage comprises water loss or dehydration. In some embodiments, the UV skin damage comprises upregulated expression of one or more target genes. In some embodiments, the UV skin damage comprises downregulated expression of one or more target genes. In some embodiments, the upregulated or downregulated expression of one or more target genes correlates with a symptom of UV skin damage. In some embodiments, the skin damage includes one or more of darkening of the skin, formation of actinic keratoses, and/or wrinkles.

Some embodiments relate to UV skin damage or suspected UV skin damage. In some embodiments, the UV skin damage or suspected UV skin damage is caused by UV exposure. In some embodiments, the UV exposure includes exposure to sunlight. In some embodiments, the UV exposure includes exposure from a synthetic UV light source. In some embodiments, the synthetic UV light source includes a laser.

### EXAMPLES

These examples are provided for illustrative purposes only and not to limit the scope of the claims provided herein.

### Example 1

This study was to determine changes in skin gene expression following exposure to ultraviolet (UV) B radiation.

Non-invasive, adhesive biopsies (DermTech) were performed on the right and left post-auricular areas of 24 subjects before and 24-hours following UV-B exposure using the excimer laser dosed at 300 mJ. RNA was isolated from the adhesive biopsies and then underwent reverse transcriptase followed by quantitative polymerase chain reaction protocols to extract DNA. Gene expression was determined 18 genes previously believed to hold a role in skin cancer development.

Of these 18 genes, 8 showed significantly changed gene expression (p<0.05) related to UV-exposure (based on T-test, comparing gene expression in skin after 24 hour UV exposure to skin before UV exposure from the same site) (see Table 3). These 8 genes include CRAPB2, IL1RN, IL36G, MUCL1, PDCD4, SPRR1A, CST6, and KLK10. One gene, UHRF1, did not demonstrate significant change in gene expression after 24 hours, but showed significant change (p<0.05) 2 weeks following UV exposure, suggesting this gene is a slow responder to UV irradiation.

These results indicate that UV exposure does cause quick gene expression change among exposed skin cells.

TABLE 3

| | | | | | Natural | |
|---|---|---|---|---|---|---|
| | | UV-Effect | ΔΔCt | | Recovery | ΔΔCt |
| | | p-val | Averag | SD | p-val | Averag |
| CRABP2 | day 1 | 0.000 | 0.728 | 0.985 | | |
| | 24 hr post UV | | | | | |
| | 2 weeks | | | | 0.185 | |
| | post UV + T4 or Ph | | | | | |
| IL1RN | day 1 | 0.000 | −1.217 | 1.928 | | |
| | 24 hr post UV | | | | | |
| | 2 weeks | | | | 0.013 | −1.399 |
| | post UV + T4 or Ph | | | | | |
| IL36G | day 1 | 0.000 | −3.279 | 4.665 | | |
| | 24 hr post UV | | | | | |
| | 2 weeks | | | | 0.000 | −4.357 |
| | post UV + T4 or Ph | | | | | |
| MUCL1 | day 1 | 0.000 | 1.925 | 1.353 | | |
| | 24 hr post UV | | | | | |
| | 2 weeks | | | | 0.002 | 1.476 |
| | post UV + T4 or Ph | | | | | |
| PDCD4 | day 1 | 0.000 | 1.251 | 1.533 | | |
| | 24 hr post UV | | | | | |
| | 2 weeks | | | | 0.000 | 1.531 |
| | post UV + T4 or Ph | | | | | |
| SPRR1A | day 1 | 0.000 | 0.821 | 1.158 | | |
| | 24 hr post UV | | | | | |
| | 2 weeks | | | | 0.340 | |
| | post UV + T4 or Ph | | | | | |
| CST6 | day 1 | 0.000 | 1.328 | 1.205 | | |
| | 24 hr post UV | | | | | |
| | 2 weeks | | | | 0.026 | 1.135 |
| | post UV + T4 or Ph | | | | | |
| KLK10 | day 1 | 0.002 | 0.571 | 1.170 | | |
| | 24 hr post UV | | | | | |
| | 2 weeks | | | | 0.515 | |
| | post UV + T4 or Ph | | | | | |

The data are normalized to Day 0, which is set as background (pre-UV exposure).

The ΔΔCt is calculated as follows:

ΔΔCt=ΔCt·post UV−ΔCt·pre UV (baseline),

where ΔCt=Ct·target gene−ΔCt·House Keeping.

A negative ΔΔCt value indicates a lower Ct·target gene expression (post UV) in the above equation (or an upregulated gene expression, which yielded a smaller Ct in qPCR).

A positive ΔΔCt value indicates a higher Ct·target gene (post UV).

### Example 2

The ability to detect and treat DNA damage remains a clinical challenge. There are intrinsic mechanisms known to repair DNA damage in bacteria, plants and some animals, but humans seem to have limited options for this process and are thought to rely mainly on nucleotide excision repair (NER) mechanisms. The use of topical DNA repair enzymes, specifically T4 Endonuclease V (T4N5) and Photolyase, may assist in the removal of cyclobutane pyrimidine dimers formed after UV irradiation. Further, these topical DNA repair enzymes may be used in the prevention of actinic keratoses and skin cancers. This study was to determine gene expression changes induced by UVB light and assess the effect of topical DNA repair enzymes in reversing these changes.

An innovation of non-invasive, adhesive skin biopsies allows for the detection of DNA damage in human skin cells at the molecular level after acute exposure to ultraviolent light. This new technology may also have the ability to monitor the recovery of DNA damage caused by UV light and the ability of topical agents to assist in this recovery process. Some embodiments described herein provide for the ability to monitor the recovery of DNA damage caused by UV light. Some embodiments provide for strategic prescription of topical agents to assist in the recovery process. Some embodiments provide for the ability to monitor the recovery of DNA damage caused by UV light and further provide for strategic prescription of topical agents to assist in the recovery process. The results of a UV skin damage test may allow recommendation of a specific sunscreen or moisturizer for prevention of further damage to, for example, topical agents, chemical peels, lasers, over-the-counter products, or prescription products, for specific treatment depending on the level of damage.

Non-invasive, adhesive patch skin biopsies were performed on the right and left post-auricular areas of 48 subjects before and 24-hours after UVB exposure using an excimer laser (300 mJ). Subjects then applied DNA repair enzymes (T4N5 endonuclease or photolyase) to the right post-auricular area only daily for 2 weeks. Subjects returned 2 weeks later for repeat biopsies. RNA was isolated and assessed by reverse transcriptase followed by quantitative PCR to assess gene expression changes.

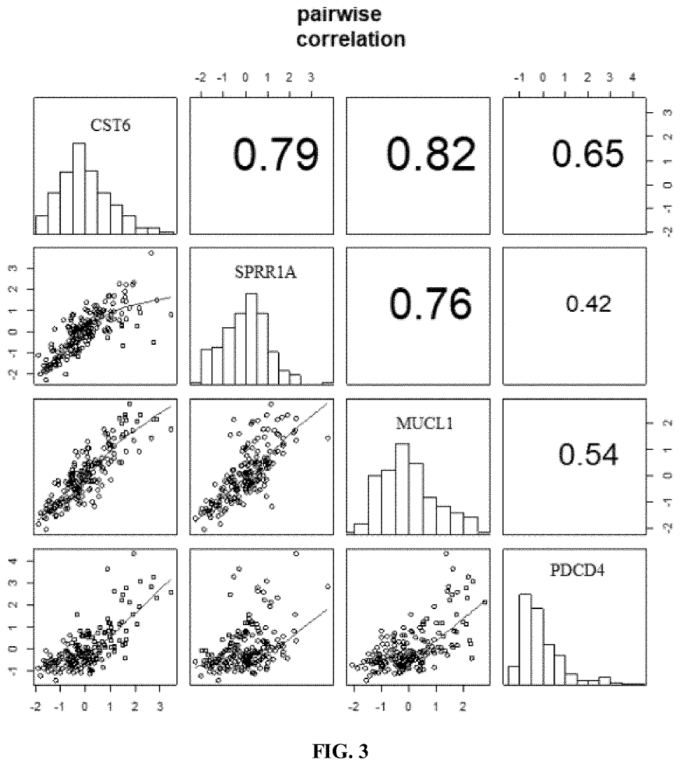

The studies in this example included skin samples from subjects aged between 22 and 89 years, from a single-site in Southern California. Both healthy volunteers and volunteers with a history of skin cancer(s) were recruited. UV exposure was carried out with the Xtrac Velocity 400 laser manufactured by PhotoMedex, Inc to illicit DNA damage at a wavelength of 308 nm on 2 areas of skin, approximately 2 cm×2 cm, on both post-auricular sides of the participant. Skin samples were collected with 8 non-invasive, adhesive patch biopsies from both post-auricular areas on the day of participant's first visit before laser treatment (time 0, to establish baseline before laser treatment), and from the same laser treated skin areas (post auricular) on the next day after laser treatment (24 hours, post laser treatment). Participants were then randomized to 2 groups, one group received T4 Endonuclease topical DNA repair enzyme, applied twice daily for 2 weeks, to the laser treated area on the right post auricular side, and the other group received photolyase creams, applied once daily for 2 weeks, to the laser treated area also on the right post auricular side. In both groups, the laser treated area from the left post auricular side received no topical treatment throughout the 2 week study (as topical treatment control). The key ingredients in the T4 Endonuclease cream include DNA enzymes and contain approximately 0.6 g of photolyase per 30 mL unit. The key ingredients in the photolyase cream include DNA enzymes (Barnet Photosomes: Plankton Extract) and contains approximately 0.3 g photolyase per 30 mL unit. Erythema and associated symptoms were graded at 24-hours following UVB exposure using the clinical questionnaire shown in FIG. 3. At the end of the 2 week topical treatment, skin samples were collected again from all participants from both the left (received laser treatment only) and right post auricular site (received both laser and topical treatment) for analysis of topical treatment effect on repairing damage caused by laser treatment.

A skin UV damage assessment method was tested. As discussed, tests included 48 male and female human test subjects, with adhesive biopsies being obtained from skin areas behind the left and right ears of the test subjects (n=48×2) (data from Example 2). Four of 7 genes that were significantly downregulated were incorporated into an algorithm and used UV exposure scores were generated. UV exposure scores from the samples matched unexpectedly well to with overall skin condition in the subjects, signifying the utility of incorporating such scores into a skin assessment method.

Total RNA was reverse transcribed to complementary DNA (cDNA) using a qPCR™ cDNA SuperMix kit from Quanta Biosciences™. The resulting cDNA was subsequently used for target gene expression analysis with TaqMan qPCR on an ABI7900 PCR system (Life Technologies). Each qPCR reaction used 30 pg of total RNA, in duplicate, in 20 uL volume on 384-well PCR reaction plates using pre-designed gene-specific TaqMan probe chemistries (Life Technologies). An averaged cycle threshold (Ct) value of the duplicate measurements was used in the analysis. A lower Ct value indicates more target gene products in the qPCR reaction or an increased gene expression in the skin sample. As Ct value from RT-qPCR is also affected by the input quantity of RNA to the reactions (more RNA input would lead to a lower Ct value would be), analysis of a housekeeping gene (human β-actin, or ACTB) is included to the test to normalize the impact of RNA input on target gene analysis. On all samples, ACTB was analyzed in qPCR in parallel with the target genes, for the calculation of ΔCt (=Ct·ACTB−Ct·target) on each target gene. As ACTB is a strongly expressed housekeeping gene, yielding a low Ct value, a smaller ΔCt value would mean a stronger expression of the target gene in test samples.

While ΔCt reflects the expression level of target gene in test samples, ΔΔCt, calculated from ΔCt from samples before and after treatment (ΔΔCt=ΔCt·target gene after treatment-ΔCt·target gene before treatment), shows how the treatment (UVB or topical DNA repair enzymes) has caused the change of target gene expression in samples following the treatment (by UVB or topical DNA repair enzymes). A negative ΔΔCt value indicates an increased gene expression (up regulation), while a positive value indicate a reduced gene expression (down regulation) caused by UVB exposure and the treatment. The fold change (FC) of gene expression was calculated as FC=2−ΔΔCt. ΔΔCt was used to show gene expression changes relating to treatment throughout the study.

Forty-six subjects completed the study (average age 49, with subjects ranging from 23 to 89 years of age). Fifteen (33%) men and 31 (67%) women. Twenty-two (48%) subjects had a history of skin cancer (13 basal cell carcinoma, 9 squamous cell carcinoma and 2 melanoma) and 24 (52%) subjects had no history of skin cancer). Some of the subjects had multiple types of skin cancer. Twenty-four (52%) subjects were randomized into the T4N5 group and 22 (48%) into the photolyase group. Erythema localized to the treatment sites was visualized clinically in 100% of subjects at 24-hours indicating sufficient UVB exposure to illicit a local reaction. All subjects were either asymptomatic or had mild burning, stinging or itching at the site of UVB exposure. At 2 weeks post-UVB exposure, there was complete resolution of erythema and other associated UVB exposure symptoms in all subjects.

Table 4 includes some data from the study. Eight out of 18 assessed genes demonstrated significant downregulation (gene families including Vitamin A, Programmed Cell Death protein, and Small Proline Rich Protein) or upregulation (Interleukin Families 1/2) 24-hours following UVB exposure. T4N5 significantly reversed UVB-induced downregulation of small proline rich protein and cystatin gene families. Photolyase significantly reversed UVB-induced downregulation of cystatin gene families.

TABLE 4

| | | | | | | | | p-val (T- |
|---|---|---|---|---|---|---|---|---|
| | | | | | | p-val (T- | p-val (T- | TEST) Test |
| | | | | | | TEST) Test | TEST) Test | vs. 2 wks |
| | | | | | | vs. Baseline | vs. 24 hrs | post UV |
| | | Ave ΔΔCt | Std | SE | N | (2 tails) | post UV | Ctrl |
| CRABP2 | Baseline (pre-UV) | 0.00 | 0.00 | 0.00 | 92 | | | |
| | 24 hrs post UV | 0.95 | 1.61 | 0.17 | 92 | 1.92E−07 | | |
| | 2 wks Post UV (Ctrl) | 0.25 | 0.94 | 0.14 | 48 | 7.84E−02 | 2.68E−03 | |
| | 2 wks Post UV and T4 | 0.15 | 0.93 | 0.21 | 21 | 4.93E−01 | 9.09E−02 | 6.72E−01 |
| | 2 wks Post UV and Pt | 0.40 | 1.11 | 0.22 | 27 | 7.41E−02 | 1.55E−01 | 5.77E−01 |
| IL1RN | Baseline (pre-UV) | 0.00 | 0.00 | 0.00 | 92 | | | |
| | 24 hrs post UV | −1.05 | 1.97 | 0.21 | 92 | 1.80E−06 | | |
| | 2 wks Post UV (Ctrl) | −0.14 | 1.27 | 0.19 | 48 | 4.81E−01 | 6.83E−03 | |
| | 2 wks Post UV and T4 | −0.12 | 1.37 | 0.31 | 21 | 7.07E−01 | 5.03E−02 | 9.61E−01 |
| | 2 wks Post UV and Pt | −0.44 | 1.63 | 0.32 | 27 | 1.71E−01 | 2.81E−01 | 4.08E−01 |
| IL36G | Baseline (pre-UV) | 0.00 | 0.00 | 0.00 | 92 | | | |
| | 24 hrs post UV | −2.33 | 4.14 | 0.43 | 92 | 5.23E−07 | | |
| | 2 wks Post UV (Ctrl) | −0.20 | 3.37 | 0.49 | 48 | 6.88E−01 | 1.14E−04 | |
| | 2 wks Post UV and T4 | −0.23 | 4.27 | 0.95 | 21 | 8.11E−01 | 4.54E−01 | 9.79E−01 |
| | 2 wks Post UV and Pt | −0.99 | 4.42 | 0.87 | 27 | 2.55E−01 | 4.76E−01 | 4.30E−01 |
| CDKN1A | Baseline (pre-UV) | 0.00 | 0.00 | 0.00 | 92 | | | |
| | 24 hrs post UV | 0.00 | 0.86 | 0.09 | 92 | 9.70E−01 | | |
| | 2 wks Post UV (Ctrl) | 0.05 | 0.57 | 0.08 | 48 | 5.73E−01 | 7.97E−01 | |
| | 2 wks Post UV and T4 | −0.05 | 0.44 | 0.10 | 21 | 6.49E−01 | 9.44E−01 | 4.74E−01 |
| | 2 wks Post UV and Pt | 0.04 | 0.54 | 0.11 | 27 | 6.70E−01 | 6.25E−01 | 9.80E−01 |
| | | | | 0.00 | | | | |
| MUCL1 | Baseline (pre-UV) | 0.00 | 0.00 | 0.00 | 92 | | | |
| | 24 hrs post UV | 2.02 | 1.73 | 0.18 | 92 | 1.01E−18 | | |
| | 2 wks Post UV (Ctrl) | 0.33 | 1.25 | 0.18 | 48 | 8.02E−02 | 2.01E−06 | |
| | 2 wks Post UV and T4 | 0.49 | 1.81 | 0.41 | 21 | 2.40E−01 | 9.60E−04 | 7.22E−01 |
| | 2 wks Post UV and Pt | 0.55 | 1.11 | 0.22 | 27 | 1.69E−02 | 2.02E−03 | 4.52E−01 |
| PDCD4 | Baseline (pre-UV) | 0.00 | 0.00 | 0.00 | 92 | | | |
| | 24 hrs post UV | 1.46 | 1.89 | 0.20 | 92 | 5.24E−11 | | |
| | 2 wks Post UV (Ctrl) | −0.03 | 0.72 | 0.11 | 48 | 7.76E−01 | 1.44E−07 | |
| | 2 wks Post UV and T4 | 0.30 | 1.59 | 0.36 | 21 | 4.05E−01 | 1.79E−03 | 3.79E−01 |
| | 2 wks Post UV and Pt | 0.30 | 1.09 | 0.21 | 27 | 1.65E−01 | 1.56E−02 | 1.69E−01 |
| SPRR1A | Baseline (pre-UV) | 0.00 | 0.00 | 0.00 | 92 | | | |
| | 24 hrs post UV | 0.81 | 1.27 | 0.13 | 92 | 2.46E−08 | | |
| | 2 wks Post UV (Ctrl) | 0.32 | 1.17 | 0.17 | 48 | 7.47E−02 | 2.63E−02 | |
| | 2 wks Post UV and T4 | −0.15 | 1.14 | 0.25 | 21 | #N/A | 3.96E−04 | 1.39E−01 |
| | 2 wks Post UV and Pt | 0.33 | 1.15 | 0.23 | 27 | 1.48E−01 | 1.48E−01 | 9.66E−01 |
| CST6 | Baseline (pre-UV) | 0.00 | 0.00 | 0.00 | 92 | | | |
| | 24 hrs post UV | 1.54 | 1.49 | 0.16 | 92 | 3.03E−16 | | |
| | 2 wks Post UV (Ctrl) | 0.15 | 0.91 | 0.13 | 48 | 2.61E−01 | 5.31E−07 | |
| | 2 wks Post UV and T4 | 0.01 | 1.34 | 0.30 | 21 | 9.63E−01 | 1.37E−05 | 6.73E−01 |
| | 2 wks Post UV and Pt | 0.25 | 1.23 | 0.24 | 27 | 2.96E−01 | 2.45E−03 | 7.23E−01 |
| KLK10 | Baseline (pre-UV) | 0.00 | 0.00 | 0.00 | 92 | | | |
| | 24 hrs post UV | 0.41 | 1.86 | 0.19 | 92 | 3.78E−02 | | |
| | 2 wks Post UV (Ctrl) | 0.21 | 1.16 | 0.17 | 48 | 2.38E−01 | 1.26E−01 | |
| | 2 wks Post UV and T4 | −0.03 | 0.87 | 0.20 | 21 | 8.97E−01 | 6.11E−02 | 3.76E−01 |
| | 2 wks Post UV and Pt | −0.41 | 2.76 | 0.54 | 27 | 4.52E−01 | 1.01E−01 | 2.81E−01 |
| CDKN2A | Baseline (pre-UV) | 0.00 | 0.00 | 0.00 | 92 | | | |
| | 24 hrs post UV | 0.89 | 3.93 | 0.41 | 92 | 1.25E−01 | | |
| | 2 wks Post UV (Ctrl) | 0.42 | 3.53 | 0.52 | 48 | 5.77E−01 | 4.66E−01 | |
| | 2 wks Post UV and T4 | 0.51 | 2.46 | 0.55 | 21 | 6.36E−01 | 2.61E−01 | 9.44E−01 |
| | 2 wks Post UV and Pt | 2.05 | 2.82 | 0.55 | 27 | 6.64E−03 | 6.81E−02 | 1.07E−01 |

Table 5 summarizes data for members of several gene families.

TABLE 5

| | Non-Limiting | 24 Hour |
|---|---|---|
| | Gene Family | UV-Effect |
| Gene Family Name | Member Example | (p-val < 0.05) |
| ADAMTSL Family | ADAMTSL4 | no |
| CDKN Family | CDKN1A | no |
| CST Family | CST6 | yes |
| KIF Family | KIF18B | no |
| MKI Family | MKI67 | no |
| SLAM Family | SLAMF7 | no |
| TRIP Family | TRIP13 | no |
| UHRF Fannily | UHRF1 | no |
| Vitamin A Family | CRABP2 | yes |
| Interleukin Family (1) | IL1RN | yes |
| Interleukin Family (2) | IL22RA1 | no |
| Interleukin Family (3) | IL36B | no |
| Interleukin Family (4) | IL36G | yes |
| KLK Family | KLK10 | yes |
| KRT Family | KRT17 | no |
| MUCL Family | MUCL1 | yes |
| PDCD Family | PDCD4 | yes |
| SPRR Family | SPRR1A | yes |

These results indicate that UVB exposure causes acute changes in gene expression and that DNA repair enzymes demonstrate efficacy in reversing these changes. Topical T4N5 and photolyase can increase cystatin gene expression following UVB-induced downregulation within 2 weeks of application. Cystatins have been reported to be diminished or lost in both basal and squamous cell carcinomas, and these findings indicate that topical DNA repair enzymes may hold the ability to repair UV-induced genetic changes.

These results demonstrate that non-invasive, adhesive skin biopsies have the ability to detect changes in gene expression following exposure to UV light and to monitor the recovery of these changes over time. Further, these results illustrate that topical DNA repair enzymes may have the ability to assist in the recovery of any UV-induced up or downregulation in gene expression.

The vitamin A, mucin like protein, programmed cell death protein, small proline rich protein, and cystatin gene families all exhibited a downregulation in gene expression at 24-hours. Interleukin gene families 1 and 2 exhibited an upregulation in gene expression at 24-hours. These results indicate that the aforementioned gene families are acutely impacted by exposure to UV light and reflects inflammation and other changes caused by UVB light exposure and sun damage.

Two weeks following exposure to UVB light, five of seven (71%) gene families tested demonstrate profound UV-induced acute changes in gene expression. These changes exhibited a reversal towards baseline, or pre-UVB exposure. Mucin like protein, programmed cell death protein, and cystatin gene families exhibited a decrease in the acute downregulation seen at 24 hours. Interleukin families 1 and 2 displayed a decrease in the acute upregulation of gene expression. These results indicate that human epidermal cells appear to be able to naturally recover from acute UV exposure over the course of 2 weeks from the gene families tested here. Additional experiments may assess the minimal amount of time needed to achieve full recovery. In addition, other gene families that may not fully recover from UVB exposure may be investigated and used to better understand genetic changes in response to UV exposure.

Application of T4N5 DNA repair enzyme cream yielded upregulation in small proline-rich proteins. This family of proteins are structural proteins that play a role in the cornified cell envelope of stratified squamous epithelial cells, functioning as barrier proteins. Following UVB exposure, downregulation of this protein family was observed, with a reduction in this downregulation following 2-weeks of natural recovery. This indicates that this topical DNA repair enzyme may not only assist in natural recovery of any UV-induced gene expression changes but also cause upregulation of these cross-bridging proteins. Photolyase yielded a reduction in the initial downregulation of mucin-like protein. Mucins are produced by epithelial cells may play a role in lubrication, cell signaling, and forming chemical barriers. In addition, both T4N5 and photolyase demonstrated reduction of the downregulation of cystatin gene expression. Cystatins may be diminished or lost in both basal and squamous cell carcinomas and these findings indicate that topical DNA repair enzymes may be able to potentially prevent the progression of UV skin damage.

These experiments also looked into the potential effects of covariates including analysis of the potential effect of sample collection site (left or right), sample analysis batch, test subjects' age, gender, skin type and skin history on gene expression response to low dose UV exposure. None of these factors affected the gene expression results.

In summary, these experiments implicate certain gene families that are altered by UV exposure, and may subsequently play a role in UV skin damage. Additionally, these experiments demonstrate that the addition of topical DNA repair enzymes may influence gene expression and DNA damage induced by UV exposure.

### Example 3

Using data from Example 2, spaghetti plots were generated for ΔCt vs. time point by batch and side (skin area behind right or left ear). Boxplots were generated for ΔΔCt by batch and side. Distribution of change was assessed for 24 hr post-UV versus pre-UV samples.pdf shows the distribution of change. Data was assessed for right and left skin areas separately, and in combination.

A linear mixed effects (LME) model was used. Because the left and right samples from the same subject are not independent, paired t-test of pre-UV versus post-UV were not used. Also, when averaging the left-side and right-side samples, it is assumed that UV and effects are the same on both left and right sides, and may underestimate the variability in the samples. LME allowed use of both the left and right samples while taking account of any dependence among the samples. A benefit of using LME models is that they can provide more accurate p-values, and may account for sources of variability than other statistical methods.

Two versions of LME models were used. Version 1 included: ΔCt time+batch+interaction (time, batch)+side+(1|subject/side). In Version 1, ΔCt was normalized Ct, subject was a random effect, face side was a random effect nested within subject, and a coefficient for time was the slope in ΔCt. Version 2 included: ΔΔCt˜batch+side+(1|subject). In version 2, ΔΔCt was the change in ΔCt 24 hr post-versus pre-UV, and subject was a random effect, so both sides could be analyzed. Version 1 takes into account random differences in ΔCt pre-UV among subjects (ΔCt at different time points and two sides within a subject), and Version 2 takes into account random differences in ΔΔCt among subjects (ΔΔCt from different sides). Variations for each version were also performed to examine the effect of batch and side. P-values were adjusted for multiple comparison (Benjamini-Hochberg).

Results from Version 1 of the LME model (based on ΔCt): when using adjusted P values <0.05 as being significant, neither batch nor side significantly affected the change in ΔCt at 24 hr post-UV vs. pre-UV for any of the genes, so examining the change in all batches combined was performed instead of examining the change within each batch. Based on the results, 7 genes out of CDKN1A, CST6, CRABP2, IL1RN, IL36G, KLK1, MUCL1, PDCD4, and SPRR1A, other than CDKN1A and KLK10, had adjusted P values <0.001. KLK10 was borderline significant with adjusted P value of 0.051. CDKN1A was not significant (adjusted P=0.97).

Results from version 2 of the LME model (based on ΔΔCt): when using adjusted P values <0.05 as being significant, neither batch nor side significantly affected ΔΔCt at 24 hr post-UV vs. pre-UV for any of genes, so examining the ΔΔCt for all batches combined was performed, instead of examining the change within each batch. Based on the results, out of 9 genes (out of CDKN1A, CST6, CRABP2, IL1RN, IL36G, KLK1, MUCL1, PDCD4, and SPRR1A) all had adjusted P values <0.001, except CDKN1A and KLK10. KLK10 and CDKN1A were not significant.

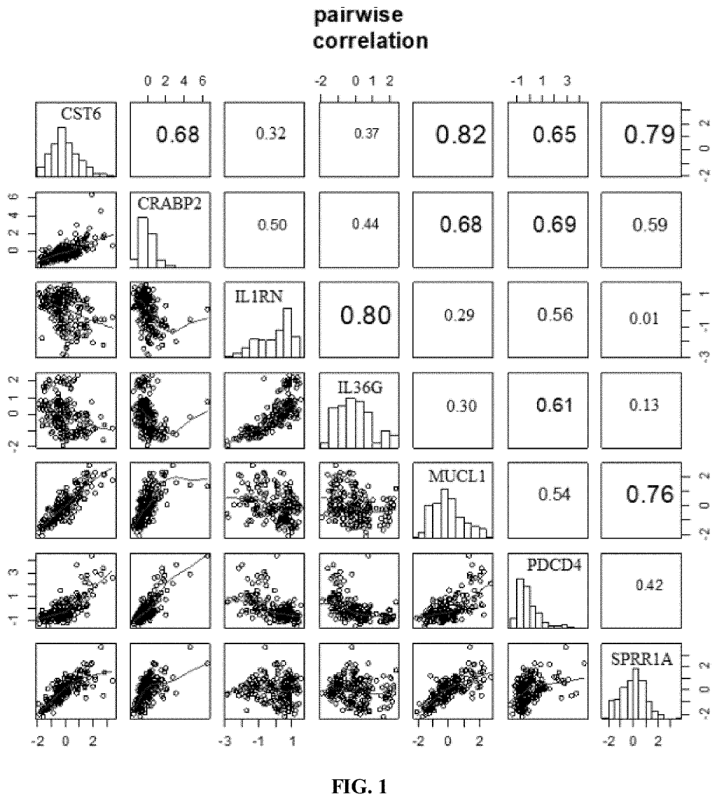

Multivariate analysis results: Ct values were standardized. Seven analytes significant in the univariate analysis were included as the predictors. Pre-UV vs. 24 hr post-UV was a dependent variable. A logistic regression model was performed. Data are shown in Table 6. Because some genes such as IL36G had small coefficients, and the correlations among some genes were high (in FIG. 1), it may be useful in a multivariate analysis to exclude such genes (some examples are underlined in Table 6). It would be useful in some instances to exclude some target genes because doing so could decrease costs in a method that uses various target genes. Some embodiments include excluding IL1RN from the multivariate analysis. Some embodiments include excluding IL36G from the multivariate analysis. Some embodiments include excluding a combination of target genes from the multivariate analysis. Some embodiments include excluding a target gene that has a low estimation value (e.g. between 0.30 and 0, between 0.25 and 0, between 0.20 and 0, between 0.15 and 0, between 0.10 and 0, between 0.05 and 0, between −0.30 and 0, between −0.25 and 0, between −0.20 and 0, between −0.15 and 0, between −0.10 and 0, or between −0.05 and 0) from the multivariate analysis. Some embodiments include excluding a target gene that has a P-value above a threshold such as 0.05 from the multivariate analysis.

TABLE 6

| Gene | Estimate | P value |
|---|---|---|
| CST6 | 1.308 | 0.024 |
| CRABP2 | −0.347 | 0.412 |
| IL1RN | −0.136 | 0.663 |
| IL36G | −0.004 | 0.989 |
| MUCL1 | 1.512 | 0.001 |
| PDCD4 | 0.969 | 0.052 |
| SPRR1A | −1.393 | 0.003 |

A multivariate analysis was performed to potentially reduce the number of variables. Results of are shown in FIG. 2 and Table 7. In this analysis, it was determined whether there were interactions of target gene on UV exposure scores, and there was no gene interaction effect.

TABLE 7

| Sequence | rf | boosting | logit | lasso |
|---|---|---|---|---|
| 1 | MUCL1 | CST6 | CST6 | CST6 |
| 2 | PDCD4 | MUCL1 | SPRR1A | SPRR1A |
| 3 | CST6 | PDCD4 | MUCL1 | MUCL1 |
| 4 | SPRR1A | CRABP2 | PDCD4 | PDCD4 |
| 5 | IL1RN | IL1RN | KLK10 | KLK10 |
| 6 | CRABP2 | IL36G | CRABP2 | batch |
| 7 | CDKN1A | SPRR1A | CDKN1A | CDKN1A |
| 8 | IL36G | CDKN1A | batch | IL1RN |
| 9 | KLK10 | KLK10 | Site | CRABP2 |
| 10 | batch | batch | IL1RN | Site |
| 11 | Site | Site | IL36G | IL36G |

Logistic regression was performed using the top 4 genes. Data are shown in FIG. 3 and Table 8. The Estimates in Table 8 are based on standardized data (correlation coefficients).

TABLE 8

| Gene | Estimate | P-value |
|---|---|---|
| CST6 | 1.362 | 0.019 |
| SPRR1A | −1.526 | 0.001 |
| MUCL1 | 1.464 | 0.001 |
| PDCD4 | 0.880 | 0.023 |

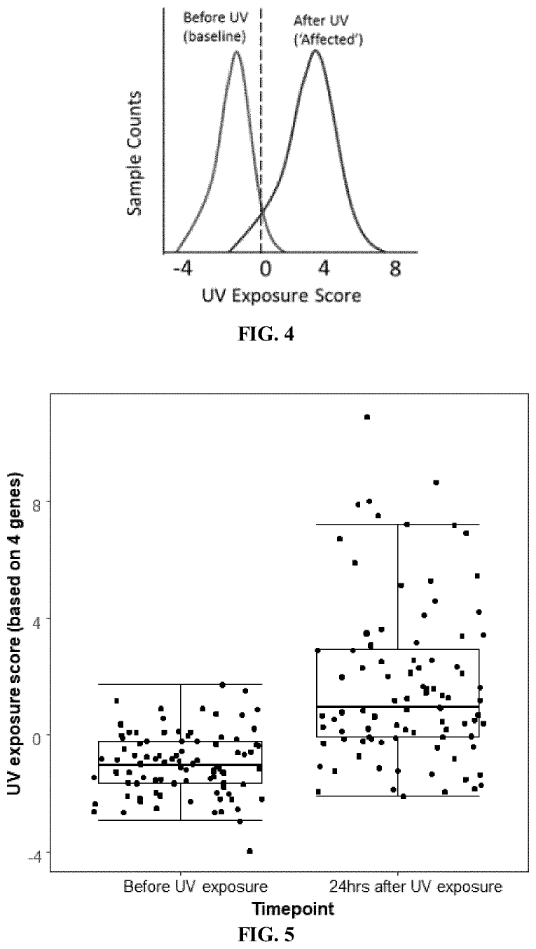

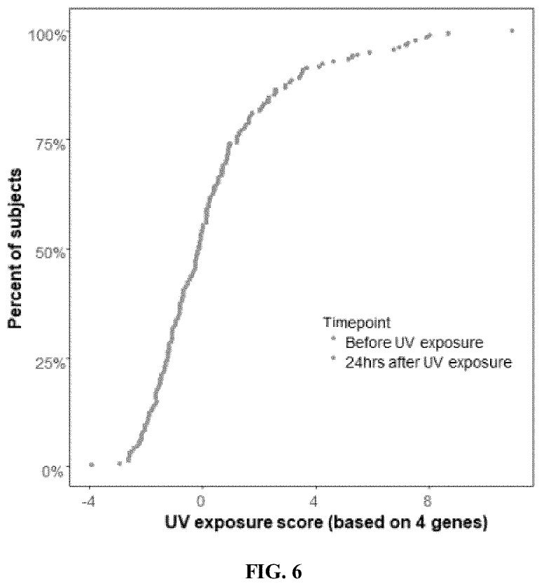

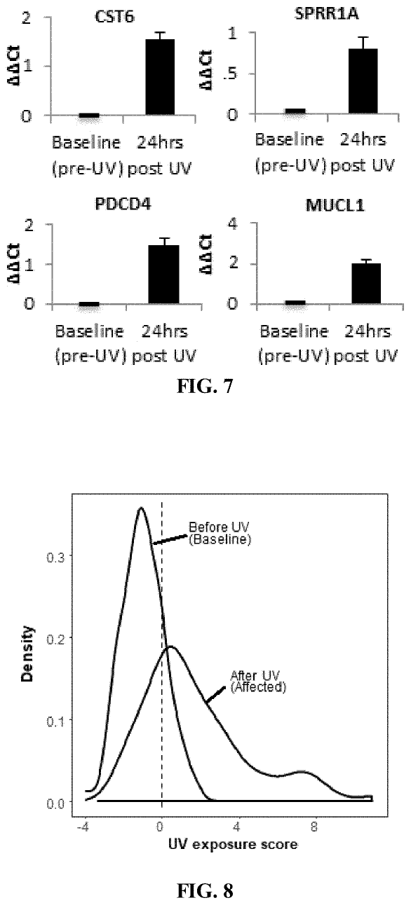

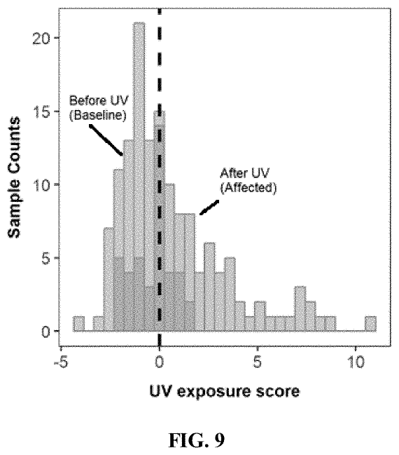

UV exposure scores were generated using the 4 target genes (CST6, SPRR1A, MUCL1, and PDCD4) through a logistic regression model. The UV exposure scores are the log-odds (probabilities on logit scale) predicted by the 4-gene logistic regression model. FIG. 4 shows a hypothetical UV exposure score distribution based on the data. FIG. 5 plots UV exposure scores for a visual comparison of the scores before and after UV exposure, and FIG. 6 shows the cumulative distribution of the UV exposure scores. FIG. 7 shows individual gene expression for the 4 genes before and after UV exposure, FIG. 8 is a density plot showing UV exposure scores before and after UV exposure, FIG. 9 is a histogram of UV exposure scores before and after UV exposure, and Table 9 shows a distribution of UV exposure scores below zero, or greater than or equal to zero for skin samples obtained before and after UV exposure. UV exposure scores from the samples matched well to the skin conditions of the subjects, thus validating the use of the UV exposure scores.

TABLE 9

| | Before UV | After UV |
|---|---|---|
| Score | (Baseline) | (Affected) |
| <0 | 74 (74%) | 26 (26%) |
| >=0 | 18 (21%) | 66 (79%) |

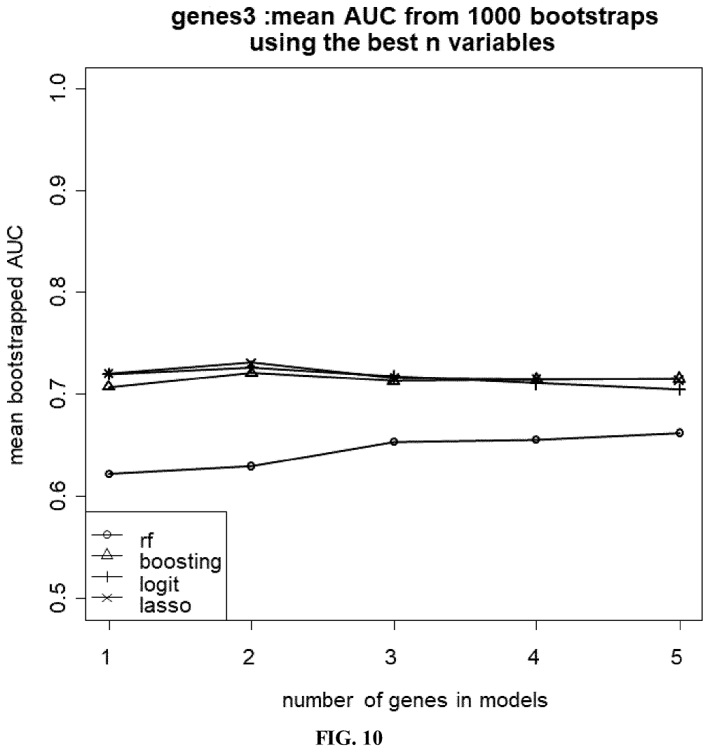

UV exposure scores were produced using various amounts of target genes. In addition, to generating UV exposure scores with a 4-gene classifier, gene classifiers were developed with 3-9 genes. Additional gene classifiers may be developed. FIG. 10 and Table 10 show multivariate analysis data for developing a 3-gene classifier.

TABLE 10

| rank | rf | boosting | logit | lasso |
|---|---|---|---|---|
| 1 | CRABP2 | CRABP2 | CRABP2 | CRABP2 |
| 2 | IL36G | IL36G | IL1RN | IL1RN |
| 3 | IL1RN | IL1RN | batch | IL36G |
| 4 | batch | batch | IL36G | batch |
| 5 | Site | Site | Site | Site |

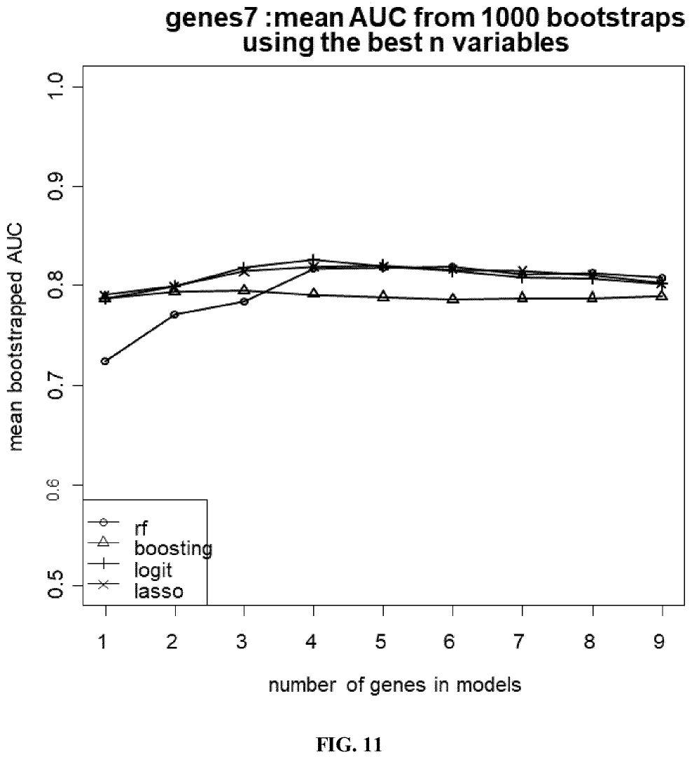

FIG. 11 and Table 11 show multivariate analysis data for developing a 3-gene classifier.

TABLE 11

| rank | rf | boosting | logit | lasso |
|---|---|---|---|---|
| 1 | MUCL1 | CST6 | CST6 | CST6 |
| 2 | PDCD4 | MUCL1 | SPRR1A | SPRR1A |
| 3 | CST6 | PDCD4 | MUCL1 | MUCL1 |
| 4 | SPRR1A | CRABP2 | PDCD4 | PDCD4 |
| 5 | IL1RN | IL1RN | CRABP2 | CRABP2 |
| 6 | CRABP2 | IL36G | IL1RN | IL1RN |
| 7 | IL36G | SPRR1A | Site | Site |
| 8 | batch | batch | batch | batch |
| 9 | Site | Site | IL36G | IL36G |

Table 12 shows univariate data.

TABLE 12

| Gene | Estimate | 95% Cl lower | 95% Cl upper |
|---|---|---|---|
| CDKN1A | 0.51 | 0.42 | 0.59 |
| CST6 | 0.79 | 0.72 | 0.85 |
| CRABP2 | 0.72 | 0.65 | 0.79 |
| IL1RN | 0.70 | 0.62 | 0.77 |
| IL36G | 0.69 | 0.61 | 0.76 |
| KLK10 | 0.60 | 0.52 | 0.68 |
| MUCL1 | 0.79 | 0.72 | 0.85 |
| PDCD4 | 0.77 | 0.70 | 0.84 |
| SPRR1A | 0.67 | 0.59 | 0.75 |

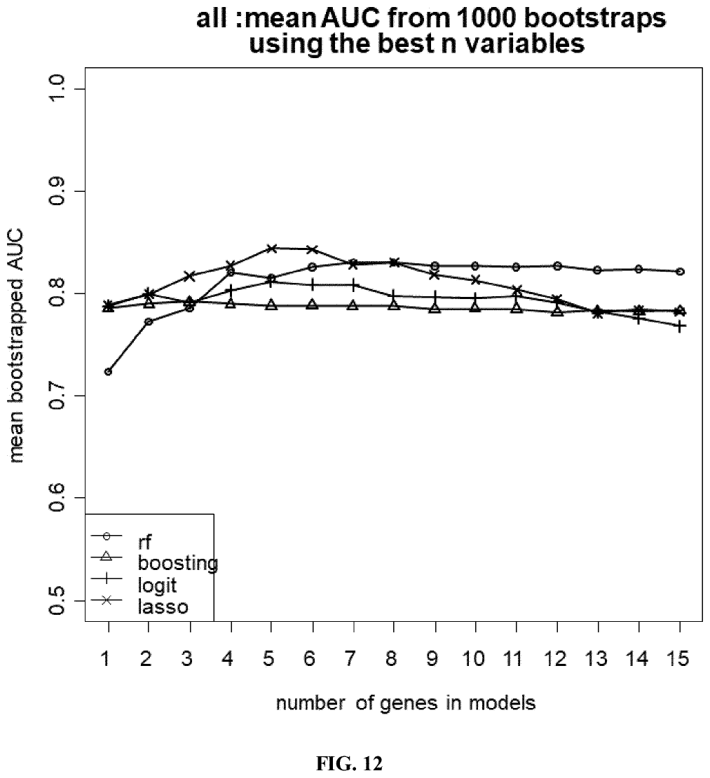

FIG. 12 and Table 13 show multivariate results including pairwise interactions among 9 genes.

TABLE 13

| rank | rf | boosting | logit | lasso |
|---|---|---|---|---|
| 1 | MUCL1 | CST6 | MUCL1 | CST6 |
| 2 | PDCD4 | MUCL1 | CST6.SPRR1A | SPRR1A |
| 3 | CST6 | PDCD4 | CRABP2.PDCD4 | MUCL1 |
| 4 | SPRR1A | CRABP2 | CST6.CRABP2 | KLK10.SPRR1A |
| 5 | IL1RN | IL1RN | CRABP2.MUCL1 | CDKN1A.PDCD4 |
| 6 | CST6.CRABP2 | IL36G | CST6.IL36G | IL36G |
| 7 | CDKN1A.SPRR1A | CST6.IL36G | IL1RN.MUCL1 | CRABP2.PDCD4 |
| 8 | CDKN1A.IL1RN | CDKN1A.IL1RN | IL36G.SPRR1A | CST6.CRABP2 |
| 9 | CST6.MUCL1 | CDKN1A.KLK10 | IL1RN.SPRR1A | CST6.IL36G |
| 10 | CRABP2 | CDKN1A.PDCD4 | CRABP2 | CST6.IL1RN |
| 11 | CDKN1A.PDCD4 | CST6.MUCL1 | MUCL1.PDCD4 | IL36G.PDCD4 |
| 12 | CDKN1A.CST6 | IL36G.MUCL1 | KLK10.SPRR1A | CST6.MUCL1 |
| 13 | CDKN1A | PDCD4.SPRR1A | CST6.KLK10 | IL1RN.IL36G |
| 14 | CST6.IL36G | CDKN1A.SPRR1A | IL36G.MUCL1 | CRABP2 |
| 15 | IL36G | CST6.CRABP2 | CST6.MUCL1 | KLK10 |

Some UV scores were produced using clinical variables in the analysis. A single-gene analysis may include any one or more of several clinical variables such as age, gender, history, and/or skin type. A single-gene analysis was produced using an LMVE model with the following formula: ΔCt time+batch+interaction (time, batch)+side+age+gender+history+skin type (1|subject/side). ΔCt was normalized Ct, subject was a random effect, and side was a random effect nested within subject. The coefficient for time was the slope in ΔCt.

Results from version 1 of the LME model (based on ΔCt): when using adjusted P values <0.05 as being significant, as before, neither batch nor side significantly affected the change in ΔCt 24 hr post-UV vs. pre-UV for any of genes, so the change in all batches combined was examined instead of examining the change within each batch. Age, gender, or skin type did not affect the change in ΔCt 24 hr post-UV vs. pre-UV for any of genes. History was significant for 10 interactions. Of 9 genes (CDKN1A, CST6, CRABP2, IL1RN, IL36G, KLK1, MUCL1, PDCD4, and SPRR1A) all but CDKN1A and KLK10 had adjusted P values <0.001

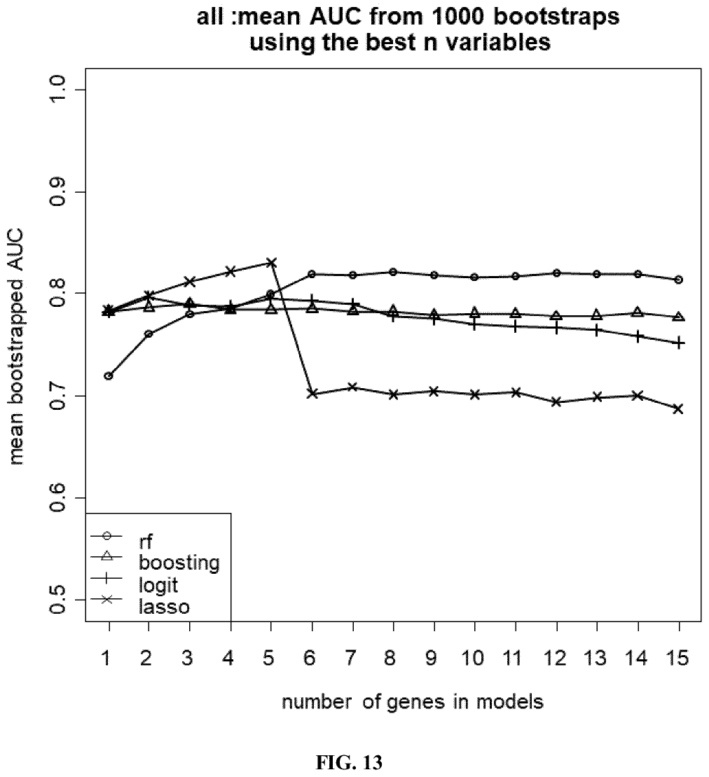

Data for a multivariate results including pairwise interactions among 9 genes (CDKN1A, CST6, CRABP2, IL1RN, IL36G, KLK1, MUCL1, PDCD4, and SPRR1A) and clinical variables are shown in FIG. 13 and Table 14. The clinical variables that were included were age, sex, history, and skin type.

TABLE 14

| rank | rf | boosting | logit | lasso |
|---|---|---|---|---|
| 1 | MUCL1 | CST6 | MUCL1 | CST6 |
| 2 | PDCD4 | MUCL1 | KLK10.SPRR1A | SPRR1A |
| 3 | CST6 | PDCD4 | CRABP2.PDCD4 | MUCL1 |
| 4 | CST6.CRABP2 | CRABP2 | CST6.CRABP2 | KLK10.SPRR1A |
| 5 | IL1RN | IL1RN | CRABP2.MUCL1 | PDCD4 |
| 6 | SPRR1A | IL36G | IL1RN.MUCL1 | Age |
| 7 | CDKN1A.IL1RN | CDKN1A.KLK10 | IL1RN.SPRR1A | CRABP2.PDCD4 |
| 8 | CDKN1A.SPRR1A | CDKN1A.IL1RN | CST6.MUCL1 | CST6.CRABP2 |
| 9 | CST6.MUCL1 | CST6.IL36G | CRABP2 | CST6.MUCL1 |
| 10 | CRABP2 | CDKN1A.PDCD4 | IL36G.MUCL1 | CDKN1A.MUCL1 |
| 11 | CDKN1A.PDCD4 | IL36G.MUCL1 | IL36G.PDCD4 | CDKN1A.SPRR1A |
| 12 | MUCL1.SPRR1A | CST6.MUCL1 | MUCL1.PDCD4 | CST6.IL36G |
| 13 | CDKN1A | CDKN1A.IL36G | IL36G.SPRR1A | KLK10.MUCL1 |
| 14 | CST6.IL36G | CDKN1A.CRABP2 | Age | CST6.IL1RN |
| 15 | IL36G | IL36G.SPRR1A | CST6.KLK10 | history |

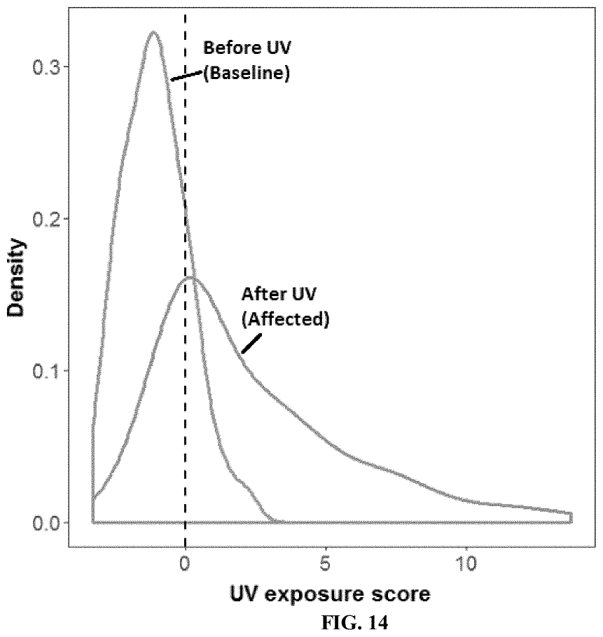

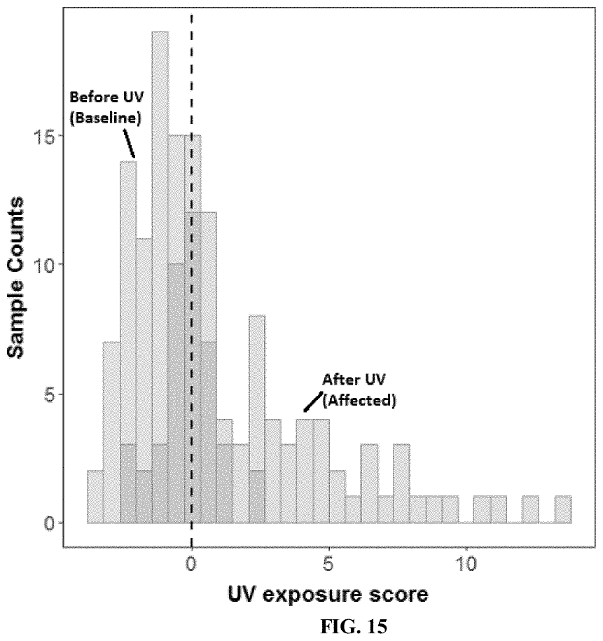

FIG. 14 and FIG. 15 show a density plot and a histogram using 5 target genes: CST6, SPRR1A, MUCL1, KLK10, SPRR1A, and PDCD4. These 5 target genes were the top 5 variables as assessed by the LASSO method gave the best AUC in FIG. 15.

Overall, multi-variable classifiers such as multi-gene classifiers and algorithms were developed that produce UV exposure scores based on severity of gene changes in response to an amount of UV radiation, and/or other variables such as age, gender, history, and/or skin type. Further tests may include collecting a new batch of samples from covered and sun exposed skins to generate UV exposure scores for further validation of the UV exposure scores, and aid in determining sun exposure. For example, additional tests may characterize UV-exposure gene scores in a cohort of patients with different skin types and weekly sun exposure levels (validating the gene scores correlated to with sun exposure levels).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of assessing ultraviolet (UV) skin damage, comprising:
identifying a subject exposed to UV radiation;
isolating nucleic acids from a skin sample obtained from the subject by applying an adhesive patch to a skin region of the subject in a manner sufficient to adhere skin sample cells to the adhesive patch, and removing the adhesive patch from the skin sample in a manner sufficient to retain the adhered skin sample cells to the adhesive patch, wherein the skin sample cells comprise cells from the stratum corneum; and
measuring or detecting an expression level of at least one target gene known to be upregulated or downregulated in subjects with UV skin damage, by contacting the isolated nucleic acids with a set of probes that recognize the target gene, and measuring or detecting binding between the at least one target gene and the set of probes.

2. The method of claim 1, wherein the UV radiation comprises UVB rays.

3. The method of claim 1, wherein the subject is a human.

4. The method of claim 1, wherein the nucleic acids comprise mRNA.

5. The method of claim 1, wherein the cells from the stratum corneum comprise keratinocytes.

6. The method of claim 1, wherein the nucleic acids are amplified prior to being contacted with the set of probes.

7. The method of claim 1, wherein the adhesive patch comprises a rubber adhesive on a polyurethane film.

8. The method of claim 1, wherein the skin sample is obtained by applying a plurality of adhesive patches to the skin region of the subject in a manner sufficient to adhere skin sample cells to each of the adhesive patches, and removing each of the plurality of adhesive patches from the skin region in a manner sufficient to retain the adhered skin sample cells to each of the adhesive patches.

9. The method of claim 1, wherein the skin region comprises a sunburn.

10. The method of claim 1, further comprising determining whether the skin sample has UV skin damage based on the expression level of the at least one target gene.

11. The method of claim 10, further comprising administering a skin treatment to the skin region of the subject based on the determination of whether the subject has UV skin damage.

12. The method of claim 11, wherein the skin treatment is topical.

13. The method of claim 11, wherein the skin treatment comprises a T4 endonuclease V-based treatment or photolyase-based treatment.

14. The method of claim 1, wherein the skin sample has UV skin damage.

15. The method of claim 1, wherein the expression level is upregulated compared to a control.

16. The method of claim 1, wherein the expression level is downregulated compared to a control.

17. The method of claim 1, wherein the at least one target gene comprises of a Vitamin A gene family or family member, a Programmed Cell Death Protein gene family or family member, a Small Proline Rich Protein gene family or family member, an Interleukin 1/2 gene family or family member, a cystatin gene family or family member, or a combination thereof.

18. The method of claim 1, wherein the expression level of genes is monitored over the course of 1 week, 2 weeks, 3 weeks, 1 month, 2 months, 6 months, or more.

19. A method of assessing ultraviolet (UV) skin damage, comprising:
obtaining expression levels of target genes in a skin sample obtained from a subject;
generating a UV exposure score for the subject by comparing the expression levels to a model derived from target gene expression levels in skin samples from a cohort of subjects, and derived from amounts UV skin damage or exposure in the cohort of subjects.

20. A method of monitoring ultraviolet (UV) skin damage, comprising:
isolating nucleic acids from a first skin sample obtained from a subject at a first time by applying an adhesive patch to a skin region of the subject in a manner sufficient to adhere skin sample cells to the adhesive patch, and removing the adhesive patch from the first skin sample in a manner sufficient to retain the adhered skin sample cells to the adhesive patch, wherein the skin sample cells comprise cells from the stratum corneum;
measuring or detecting an expression level of one or more target genes known to be upregulated or downregulated in subjects with UV skin damage, in the first skin sample;
determining a presence or an amount of UV skin damage in the first skin sample based on the expression level of the one or more target genes;
isolating nucleic acids from a skin sample obtained from the subject at a second time;
measuring or detecting an expression level of the one or more target genes in the second skin sample;
determining a presence or an amount of UV skin damage in the second skin sample based on the expression level of the one or more target genes; and
comparing the presence or amount of UV skin damage in the second skin sample to the presence or amount of UV skin damage in the first skin sample.
